(19) United States
(12) Patent Application Publication
(10) Pub. No.: US 2024/0212818 A1
(43) Pub. Date: Jun. 27, 2024

(54) SYSTEMS AND METHODS FOR MANAGING PREDIABETES WITH A GLIFLOZIN SODIUMGLUCOSE COTRANSPORT 2 INHIBITOR PHARMACEUTICAL COMPOSITION

(71) Applicant: AstraZeneca UK Limited, Cambridge (GB)

(72) Inventors: Judy FIROR, Wilmington, DE (US); Howard Hutchinson, Wilmington, DE (US); William Mongan, Wilmington, CA (US); Richard L. Skelly, Wilmington, CA (US); Jerry Valentine, Wilmington, CA (US); Merrill Zavod, Wilmington, CA (US)

(21) Appl. No.: 18/553,400

(22) PCT Filed: Apr. 1, 2022
(86) PCT No.: PCT/IB2022/000187

§ 371 Date: Sep. 29, 2023

Related U.S. Application Data
(60) Provisional application No. 63/169,729, filed on Apr. 1, 2021.

(51) Int. Cl. G16H 20/10; G16H 10/60

(52) U.S. Cl. CPC G16H 20/10; G16H 10/60

(57) ABSTRACT

A method is provided for lowering blood sugar in a subject in need thereof by administering a gliflozin Sodium-Glucose Cotransport 2 inhibitor pharmaceutical composition to a subject qualified for over-the-counter access to the gliflozin Sodium-Glucose Cotransport 2 inhibitor pharmaceutical composition. In some embodiments, the gliflozin Sodium-Glucose Cotransport 2 inhibitor pharmaceutical composition includes empagliflozin, canagliflozin, and ertugliflozin. In some embodiments, the gliflozin Sodium-Glucose Cotransport 2 inhibitor pharmaceutical composition includes (2S,3R,4R,5S,6R)-2-[4-chloro-3-[(4-ethoxyphenyl)methyl]phenyl]-6-(hydroxymethyl)oxane-3,4,5-triol or a pharmaceutically acceptable salt thereof.

### TECHNICAL FIELD

The present disclosure relates generally to methods for treating blood sugar, e.g., thereby treating prediabetes and/or delaying onset of diabetes, by administering an over-the-counter gliflozin Sodium-Glucose Cotransport 2 inhibitor pharmaceutical composition to a subject in need thereof, who has been qualified for over-the-counter access to the composition.

### BACKGROUND

Nearly a quarter of young adults, ages 19 to 34, and a fifth of adolescents, ages 12 to 18, in the U.S. have prediabetes, a precursor to type 2 diabetes. Diabetes is a leading cause of death and increasing health care costs worldwide. NCD Risk Factor Collaboration, The Lancet, 387:1513-1530 (2016). Since 1980, the number of people living with diabetes worldwide has nearly quadrupled. Id. As of 2015, according to the CDC, about 12% of all adults in the United States had diabetes and nearly 35% of all adults in the U.S. had prediabetes. Centers for Disease Control and Prevention, ‘National Diabetes Statistics Report 2017’ (2017). Further, nearly half of diabetes patients do not have their blood sugar under control. Polonsky et al., Patient Prefer. Adherence, 10:1299-1307 (2016). Moreover, diabetes poses a significant economical challenge. The American Diabetes Association estimated that in 2012, $245 billion was spent in direct and indirect medical expenses relating to diagnosed diabetes in the U.S. American Diabetes Association, Diabetes Care, 36(4):1033-46 (2013).

Fortunately, prediabetes can be managed by, for example, using gliflozin Sodium-Glucose Cotransport 2 inhibitors, which are well established prescription pharmaceuticals used for lowering blood sugar, e.g., thereby treating Type 2 diabetes and/or maintaining sub-diabetic blood sugar levels. For instance, the efficacy of dapagliflozin, which was first approved in the U.S. for the treatment of diabetes in 2014, to lower blood sugar has been demonstrated in at least 14 double-blind, placebo-controlled, randomized studies. However, access to gliflozin Sodium-Glucose Cotransport 2 inhibitors is restricted by the requirement for a prescription. Unfortunately, long-term trends demonstrate many people avoid prescription medications, including gliflozin Sodium-Glucose Cotransport 2 inhibitors.

One approach to making gliflozin Sodium-Glucose Cotransport 2 inhibitors more accessible is to make then available without a prescription, e.g., over-the-counter (“OTC”). There are a variety of health benefits derived from switching a drug from prescription to OTC including, but not limited to, generating wider availably to therapies, providing a greater number of therapeutic approaches, providing direct and rapid access to treatments, providing patients with an active role in their own health care, and allowing patients to become self-reliant in preventing and relieving minor symptoms or conditions World Health Organization, “Guidelines for the Regulatory Assessment of Medicinal Products for use in Self-Medication,” 2000. Given the large number of individuals with uncontrolled high blood sugar, providing access to OTC gliflozin Sodium-Glucose Cotransport 2 inhibitors could provide significant societal health benefits.

However, switching distribution of a pharmaceutical from prescription-only to OTC creates a significant risk that the patient population will be unable to appropriately self-select themselves for safe use of the pharmaceutical and then self-medicate using the drug in a responsible manner. The manifestations embodied within these concerns include incorrect self-diagnosis, incorrect drug-qualification, unrecognized drug-drug interactions (DDI), unanticipated adverse drug reactions and/or side-effects, improper dosing and/or administration, masking of a disease, addiction, inappropriate drug dependency, substance abuse, and patient delay in seeking necessary medical attention. Ruiz et al., Current Drug Safety, 5(4):315 (2010).

Because gliflozin Sodium-Glucose Cotransport 2 inhibitors cause adverse effects in certain patients, the population receiving the drug should be carefully selected and monitored. In order to ensure the safety of OTC distribution of gliflozin Sodium-Glucose Cotransport 2 inhibitor, prospective patients must effectively self-select themselves for the drug. Recent studies, however, found that many prospective patients do not pay consistent attention to guidelines printed on the packaging of OTC drugs, to ensure safe and responsible use. PR Newswire Association, “Americans Should Pay More Attention to Over-the-Counter (OTC) medicine Labels According to New Survey,” Oct. 15 2015 (citing McNeil Consumer Healthcare research). According to these studies, 40% of prospective patients consider the directions as just guidelines and 80% of patients do not re-read the label of an OTC medicine they have used before. Even more troubling, only 58% of men surveyed found it very important to pay attention to restrictions on an OTC label.

Currently, there are two regulatory pathways for legal marketing of an OTC drug in the United States. In the first pathway, marketing occurs in compliance with an OTC drug monograph, that sets regulatory standards for non-prescription drugs that are not covered by human drug applications, e.g., a New Drug Application (NDA) or Abbreviated New Drug Application (ANDA). An OTC monograph is created as a result of a three phase OTC drug review by the FDA. In phase I of the review, an advisory review panel determines whether ingredients in the proposed OTC composition could be generally recognized as safe and effective for use in self-treatment. In the second pathway, marketing occurs under the authority of an approved product-specific new drug application (NDA), or an abbreviated new drug application (ANDA). In order to support an over-the-counter label for a drug for which regulatory approval is being sought through an NDA, a consumer research study is required to assess the consumer's ability to select and deselect themselves as appropriate users of the drug, based on the proposed labeling for the drug. Oliver, A., Regulatory Rapporteur, 10(3):4-9 (2013), which is incorporated by reference herein.

However, attempts at switching distribution of drugs having potentially far-reaching benefits for societal health, from prescription-only to an OTC model, have repeatedly failed, in large part due to concerns over inappropriate patient selection and medication. Possibly the best documented cases relate to statins used to treat high cholesterol.

For instance, Merck has had at least three applications for sale of over-the-counter lovastatin rejected by the FDA, in 2000, 2005, and 2007. In 2005, their proposal to permit over-the-counter sales of lovastatin was rejected by an expert advisory panel at the FDA in 2005. The panel was concerned by a marketing study performed to support the proposal in which approximately one third of 3316 customers who were offered the drug over-the-counter decided they would purchase the drug. After reviewing the data, the panel concluded that 45% of the purchases would have been inappropriate for a variety of reasons, including the age of the subject, the subject's lack of knowledge about their condition, and contraindications associated with their condition. Dyer et al., BMJ, 330(7484):164 (2005). In 2007, the board again concluded that the ability of consumers to appropriately self-select and to adequately comply with chronic MEVACOR® therapy without the intervention of a physician had not been demonstrated. Division of Metabolic and Endocrine Drug Products, 2005, “NDA 21-213 Non-prescription MEVACOR® 20 mg Joint Advisory Committee Meeting.”

Similarly, Pfizer announced in 2011 its intention to switch LIPITOR® from prescription-only to OTC status. Sett OTC bulletin, 16 Nov. 2011, page 7. However, they abandoned their attempt in 2014 when a phase 3 “actual use” trial, intended to simulate the OTC use of LIPITOR® (atorvastatin calcium) 10 mg, failed to meet its primary objectives on the basis that patient compliance with the direction to check their low-density lipoprotein cholesterol (LDL-C) level and, after checking their LDL-C level, take appropriate action based on their test results was unsatisfactory. Pfizer Inc., “Pfizer Reports Second-Quarter 2015 Results,” (2015).

In fact, in the nearly two decades since Bristol-Myers Squibb and Merck & Co first failed in their attempts to switch PRAVACHOL® and lovastatin, respectively, to OTC, a statin has never been granted OTC status in the United States. This is despite that nearly ⅙th of the adult population in the U.S. is eligible for cholesterol-lowering medications, under the current guidelines, but are not taking anything.

The information disclosed in this Background section is only for enhancement of understanding of the general background of the invention and should not be taken as an acknowledgment or any form of suggestion that this information forms the prior art already known to a person skilled in the art.

### SUMMARY

Given the above background, what is needed in the art are systems and methods for qualifying a human subject for delivery of a gliflozin Sodium-Glucose Cotransport 2 inhibitor pharmaceutical composition over-the-counter to lower blood sugar, e.g., thereby treating prediabetes, and/or delaying onset of diabetes.

The present disclosure addresses the need in the art for systems and methods configured for qualifying a human subject for over-the-counter delivery of a gliflozin Sodium-Glucose Cotransport 2 inhibitor pharmaceutical composition (e.g., dapagliflozin) in order to manage prediabetes, e.g., by lowering blood sugar. In the present disclosure, systems and methods are provided for over-the-counter delivery of a gliflozin Sodium-Glucose Cotransport 2 inhibitor pharmaceutical composition to a subject.

In one aspect, methods, as well as systems and computer readable medium for performing all or portions of such methods, are provided for authorizing an over-the-counter provision of a gliflozin Sodium-Glucose Cotransport 2 inhibitor pharmaceutical composition. An assessment information set about the subject are run against a first set of assessment filters. When a filter in the first set of assessment filters is fired, the subject is deemed not qualified for delivery of the gliflozin Sodium-Glucose Cotransport 2 inhibitor pharmaceutical composition. The survey results are also run against a second set of assessment filters. When a respective filter in the second set of assessment filters is fired, the subject is provided with a corresponding warning. The method proceeds to a fulfillment process when no filter in the first set of assessment filters is fired and the subject has acknowledged each warning associated with each fired filter in the second set of assessment filters. The fulfillment process includes storing an indication in a subject profile of an initial authorization for the over-the-counter provision of the gliflozin Sodium-Glucose Cotransport 2 inhibitor pharmaceutical composition and authorizing the over-the-counter provision of the gliflozin Sodium-Glucose Cotransport 2 inhibitor pharmaceutical composition to the subject. In some embodiments, the fulfillment process communicates a drug facts label for the gliflozin Sodium-Glucose Cotransport 2 inhibitor pharmaceutical composition to the subject, and authorizes, upon subject confirmation that the label has been read, the provision of the gliflozin Sodium-Glucose Cotransport 2 inhibitor pharmaceutical composition to the subject.

In one aspect, methods, as well as systems and computer readable medium for performing all or portions of such methods, are provided for authorizing a human subject for an over-the-counter provision of a gliflozin Sodium-Glucose Cotransport 2 inhibitor pharmaceutical composition. An assessment information set about the subject is run against a plurality of assessment filters. When a respective filter in the plurality of filters is fired, the method is terminated or the subject is provided with a warning corresponding to the respective filter. The method proceeds to a fulfillment process when the method is not already terminated by the firing of a filter in the plurality of filters and, if a respective filter in the plurality of filters has been fired, acknowledgment has been received from the subject for each warning issued to the subject by any filter in the plurality of filters.

That is, the plurality of filters against which the assessment set about the subject is run includes filters of a first type that will terminate the method without authorizing the over-the-counter provision of the gliflozin Sodium-Glucose Cotransport 2 inhibitor pharmaceutical composition to the subject when fired and and/or filters of a second type that will provide the subject with a warning corresponding to the respective filter when fired. In order for the method to proceed to a fulfillment process, no filters of the first type can have already terminated the process as a result of being fired and, if any filters of the second type are fired, the system must have already received confirmation from the subject of the subsequent warning caused by the firing of that second type of filter.

In some embodiments, the plurality of filters against which the assessment set about the subject is run includes at least one filter of the first type (that will terminate the method without authorizing the over-the-counter provision of the gliflozin Sodium-Glucose Cotransport 2 inhibitor pharmaceutical composition to the subject when fired) and at least one filter of the second type (that will issue a warning to the subject when fired).

In some embodiments, the plurality of filters against which the assessment set about the subject is run includes at least one filter of the first type (that will terminate the method without authorizing the over-the-counter provision of the gliflozin Sodium-Glucose Cotransport 2 inhibitor pharmaceutical composition to the subject when fired) and at least two, three, four, five, or more filters of the second type (that will issue a warning to the subject when fired).

In some embodiments, the plurality of filters against which the assessment set about the subject is run includes at least one filter of the first type (that will terminate the method without authorizing the over-the-counter provision of the gliflozin Sodium-Glucose Cotransport 2 inhibitor pharmaceutical composition to the subject when fired) and less than five, four, three, or two filters of the second type (that will issue a warning to the subject when fired).

The fulfillment process includes storing an indication in a subject profile of an initial authorization for the over-the-counter provision of the gliflozin Sodium-Glucose Cotransport 2 inhibitor pharmaceutical composition and authorizing the over-the-counter provision of the gliflozin Sodium-Glucose Cotransport 2 inhibitor pharmaceutical composition to the subject. In some embodiments, the fulfillment process communicates a drug facts label for the gliflozin Sodium-Glucose Cotransport 2 inhibitor pharmaceutical composition to the subject, and authorizes, upon subject confirmation that the label has been read, the provision of the gliflozin Sodium-Glucose Cotransport 2 inhibitor pharmaceutical composition to the subject.

In some embodiments of the assessment methods described above, the assessment information set includes one or more pieces of information about the subject selected from an age of the subject, a gender of the subject, whether the subject is pregnant or breastfeeding (when the subject is a female), whether the subject has type I or type II diabetes, whether the subject is taking a diabetes medication, an indication of whether the subject has kidney disease, an indication of the subject's blood glucose level (e.g., HbA1c, eAG, or fasting glucose level), and an indication of whether the subject has a history of urinary tract infections. In some embodiments, the assessment information set includes all the pieces of information about the subject described above.

In some embodiments of the assessment methods described above, the first set of assessment filters includes one or more filters selected from an age assessment filter that is fired at least when the assessment information set indicates the age of the subject fails to satisfy an age threshold for receiving the gliflozin Sodium-Glucose Cotransport 2 inhibitor pharmaceutical composition, a pregnancy assessment filter (when the subject is a female) that is fired at least when the assessment information set indicates the subject is pregnant or breastfeeding, a diabetes assessment filter that is fired at least when the assessment information set indicates the subject has type 1 or type 2 diabetes, a diabetes medication assessment filter that is fired at least when the assessment information set indicates the subject is taking a diabetes medication, a kidney disease assessment filter that is fired at least when the assessment information set indicates that the subject has kidney disease, and a blood glucose assessment filter that is fired at least when the assessment information set indicates that the subject does not have prediabetes. In some embodiments, the first set of assessment filters includes all the filters described above.

In some embodiments of the assessment methods described above, the second set of assessment filters includes a urinary tract infection assessment filter that is fired at least when the assessment information set indicates that the subject has a history of urinary tract infections. In some embodiments of the assessment methods described above, the second set of assessment filters consists of a single filter: a urinary tract infection assessment filter that is fired at least when the assessment information set indicates that the subject has a history of urinary tract infections.

In some embodiments of the assessment methods described above, the plurality of assessment filters includes one or more filters selected from an age assessment filter that is fired at least when the assessment information set indicates the age of the subject fails to satisfy an age threshold for receiving the gliflozin Sodium-Glucose Cotransport 2 inhibitor pharmaceutical composition, a pregnancy assessment filter (when the subject is a female) that is fired at least when the assessment information set indicates the subject is pregnant or breastfeeding, a diabetes assessment filter that is fired at least when the assessment information set indicates the subject has type 1 or type 2 diabetes, a diabetes medication assessment filter that is fired at least when the assessment information set indicates the subject is taking a diabetes medication, a kidney disease assessment filter that is fired at least when the assessment information set indicates that the subject has kidney disease, a blood glucose assessment filter that is fired at least when the assessment information set indicates that the subject does not have prediabetes, and a urinary tract infection assessment filter that is fired at least when the assessment information set indicates that the subject has a history of urinary tract infections. In some embodiments, the plurality of assessment filters includes all the filters described above.

In some embodiments of the assessment methods described above, the method also includes administering the gliflozin Sodium-Glucose Cotransport 2 inhibitor pharmaceutical composition to the subject, upon authorization of the provision.

In one aspect, methods, as well as systems and computer readable medium for performing all or portions of such methods, are provided for re-authorizing a human subject who was previously authorized for an over-the-counter provision of a gliflozin Sodium-Glucose Cotransport 2 inhibitor pharmaceutical composition. The method includes receiving a re-order request from the subject for the gliflozin Sodium-Glucose Cotransport 2 inhibitor pharmaceutical composition. The method also includes obtaining a reassessment information set about the subject, e.g., by prompting the user to provide relevant information about themselves. An algorithm is applied to the reassessment information set. The algorithm runs all or a portion of the reassessment information set against a first set of reassessment filters. When a respective filter in the first set of assessment filters is fired, the subject is deemed not qualified for the over-the-counter provision of the gliflozin Sodium-Glucose Cotransport 2 inhibitor pharmaceutical composition, and the method is terminated without authorizing provision of the gliflozin Sodium-Glucose Cotransport 2 inhibitor pharmaceutical composition to the subject. The method proceeds to a fulfillment process when no filter in the first set of assessment filters is fired. The fulfillment process includes storing an indication in a subject profile of a reauthorization for the over-the-counter provision of the gliflozin Sodium-Glucose Cotransport 2 inhibitor pharmaceutical composition and authorizing the over-the-counter provision of the gliflozin Sodium-Glucose Cotransport 2 inhibitor pharmaceutical composition to the subject. In some embodiments, the fulfillment process communicates a drug facts label for the gliflozin Sodium-Glucose Cotransport 2 inhibitor pharmaceutical composition to the subject, and authorizes, upon subject confirmation that the label has been read, the provision of the gliflozin Sodium-Glucose Cotransport 2 inhibitor pharmaceutical composition to the subject.

In one aspect, methods, as well as systems and computer readable medium for performing all or portions of such methods, are provided for re-authorizing a human subject who was previously authorized for an over-the-counter provision of a gliflozin Sodium-Glucose Cotransport 2 inhibitor pharmaceutical composition. The method includes receiving a re-order request from the subject for the gliflozin Sodium-Glucose Cotransport 2 inhibitor pharmaceutical composition. The method also includes obtaining a reassessment information set about the subject, e.g., by prompting the user to provide relevant information about themselves. An algorithm is applied to the reassessment information set. The algorithm runs all or a portion of the reassessment information set against a first set of reassessment filters. When a respective filter in the first set of reassessment filters is fired, the method is terminated without authorizing the over-the-counter provision of the gliflozin Sodium-Glucose Cotransport 2 inhibitor pharmaceutical composition to the subject or the subject is provided with a warning corresponding to the respective filter. The method proceeds to a fulfillment process when the method is not already terminated by the firing of a filter in the first set of reassessment filters and the subject has acknowledged each warning associated with each filter in the first set of reassessment filters that was fired and that is associated with a warning. The fulfillment process includes storing an indication in a subject profile of a reauthorization for the over-the-counter provision of the gliflozin Sodium-Glucose Cotransport 2 inhibitor pharmaceutical composition and authorizing the over-the-counter provision of the gliflozin Sodium-Glucose Cotransport 2 inhibitor pharmaceutical composition to the subject. In some embodiments, the fulfillment process communicates a drug facts label for the gliflozin Sodium-Glucose Cotransport 2 inhibitor pharmaceutical composition to the subject, and authorizes, upon subject confirmation that the label has been read, the provision of the gliflozin Sodium-Glucose Cotransport 2 inhibitor pharmaceutical composition to the subject.

In some embodiments of the reassessment methods described above, the reassessment information set includes one or more pieces of information about the subject selected from whether the subject is pregnant or breastfeeding (when the subject is female), whether the subject has started taking a diabetes medication other than the over-the-counter provision of the gliflozin Sodium-Glucose Cotransport 2 inhibitor pharmaceutical composition since receiving their last over-the-counter provision of the gliflozin Sodium-Glucose Cotransport 2 inhibitor pharmaceutical composition, an indication of whether the subject has developed kidney disease since receiving their last over-the-counter provision of the gliflozin Sodium-Glucose Cotransport 2 inhibitor pharmaceutical composition, an indication of whether the subject has experienced a urinary tract infection since receiving their last over-the-counter provision of the gliflozin Sodium-Glucose Cotransport 2 inhibitor pharmaceutical composition, and an indication of whether the subject has experienced a bacterial infection since receiving their last over-the-counter provision of the gliflozin Sodium-Glucose Cotransport 2 inhibitor pharmaceutical composition. In some embodiments, the assessment information set includes all the pieces of information about the subject described above.

In some embodiments of the reassessment methods described above, the first set of reassessment filters includes one or more filters selected from a pregnancy assessment filter (when the subject is a female) that is fired at least when the assessment information set indicates the subject is pregnant or breastfeeding, a diabetes medication reassessment filter that is fired at least when the reassessment information set indicates the subject has started taking a medication for treating diabetes other than the gliflozin Sodium-Glucose Cotransport 2 inhibitor pharmaceutical composition since receiving their last over-the-counter provision of the gliflozin Sodium-Glucose Cotransport 2 inhibitor pharmaceutical composition, a kidney disease reassessment filter that is fired at least when the reassessment information set indicates that the subject has developed kidney disease since receiving their last over-the-counter provision of the gliflozin Sodium-Glucose Cotransport 2 inhibitor pharmaceutical composition, a urinary tract infection reassessment filter that is fired at least when the reassessment information set indicates that the subject has experienced symptoms of a urinary tract infection since receiving their last over-the-counter provision of the gliflozin Sodium-Glucose Cotransport 2 inhibitor pharmaceutical composition, and a bacterial infection reassessment filter that is fired at least when the reassessment information set indicates that the subject has experienced symptoms of a bacterial infection since receiving their last over-the-counter provision of the gliflozin Sodium-Glucose Cotransport 2 inhibitor pharmaceutical composition. In some embodiments, the first set of reassessment filters includes all the filters described above.

In some embodiments of the reassessment methods described above, the method also includes administering the gliflozin Sodium-Glucose Cotransport 2 inhibitor pharmaceutical composition to the subject, upon reauthorization of the provision.

Another aspect of the present disclosure is an electronic device for performing any of the embodiments described herein. The electronic device includes one or more processors, and a memory storing one or more programs configured to be executed by the one or more processors. The one or more programs include instructions for performing a method according to any of the embodiments described herein. In some embodiments, the one or more programs include instructions for performing a method for authorizing a human subject for an over-the-counter provision of a gliflozin Sodium-Glucose Cotransport 2 inhibitor pharmaceutical composition according to any of the embodiments described herein In some embodiments, the one or more programs include instructions for performing a method for reauthorizing a human subject for an over-the-counter provision of a gliflozin Sodium-Glucose Cotransport 2 inhibitor pharmaceutical composition according to any of the embodiments described herein.

Another aspect of the present disclosure provides computer readable storage medium having stored thereon one or more programs comprising instructions that, when executed by an electronic device, cause the device to perform a method according any of the embodiments described herein. In some embodiments, the one or more programs include first instructions, which when executed by an electronic device, cause the device to perform a method for authorizing a human subject for an over-the-counter provision of a gliflozin Sodium-Glucose Cotransport 2 inhibitor pharmaceutical composition according to any of the embodiments described herein. In some embodiments, the one or more programs also include second instructions, which when executed by an electronic device, cause the device to perform a method for reauthorizing a human subject for an over-the-counter provision of a gliflozin Sodium-Glucose Cotransport 2 inhibitor pharmaceutical composition according to any of the embodiments described herein.

### BRIEF DESCRIPTION OF THE DRAWINGS

FIG. 1 illustrates an example system topology that includes a gliflozin Sodium-Glucose Cotransport 2 inhibitor pharmaceutical composition over-the-counter (OTC) authorization device for qualifying a human subject for over-the-counter delivery of a gliflozin Sodium-Glucose Cotransport 2 inhibitor pharmaceutical composition, e.g., for treating prediabetes and/or delaying onset of diabetes, a data collection device for collecting subject data, one or more user devices associated with human subjects, and one or more dispensary destinations for distributing the gliflozin Sodium-Glucose Cotransport 2 inhibitor pharmaceutical composition over-the-counter, where the above-identified components are interconnected, optionally through a communications network, in accordance with various embodiments of the present disclosure.

FIG. 2 illustrates an example device for authorizing a subject for an over-the-counter provision of a gliflozin Sodium-Glucose Cotransport 2 inhibitor pharmaceutical composition, in accordance with various embodiments of the present disclosure.

FIGS. 3A, 3B, 3C, and 3D illustrate example devices associated with a human subject for qualifying the human subject for over-the-counter delivery of a gliflozin Sodium-Glucose Cotransport 2 inhibitor pharmaceutical composition, e.g., for treating prediabetes and/or delaying onset of diabetes, in accordance with various embodiments of the present disclosure. For example, FIG. 3A illustrates an example device for performing an assessment or reassessment to authorize or reauthorize the human subject for a provision of a gliflozin Sodium-Glucose Cotransport 2 inhibitor pharmaceutical composition. Similarly, FIGS. 3B, 3C, and 3D each illustrate example assessment and reassessment modules for use in a device such as the devices shown in FIGS. 1-3A, to authorize or reauthorize a human subject for an over-the-counter provision of a gliflozin Sodium-Glucose Cotransport 2 inhibitor pharmaceutical composition. It will be appreciated that in some embodiments, the example devices of FIG. 3 work in conjunction with the example device of FIG. 2 to perform the methods illustrated in FIGS. 4-8, for instance, by providing the device of FIG. 2 with survey results (e.g., assessment information sets and/or reassessment information sets) and/or the results of firing filters of the present disclosure against such survey results. However, in alternative embodiments, the device of FIG. 2 performs all steps of the methods described herein and the devices of FIG. 3 are not used. In still further alternative embodiments, various combinations of the devices of FIG. 3 perform all steps of the methods described herein and the device of FIG. 2 is not used.

FIG. 4A provides a flow chart of an example process for qualifying a human subject for over-the-counter delivery of a gliflozin Sodium-Glucose Cotransport 2 inhibitor pharmaceutical composition to lower blood sugar, where elements in dashed boxes are optional, in accordance with various embodiments of the present disclosure.

FIG. 4B provides a flow chart of an example process for requalifying a human subject for over-the-counter delivery of a gliflozin Sodium-Glucose Cotransport 2 inhibitor pharmaceutical composition to lower blood sugar, where elements in dashed boxes are optional, in accordance with various embodiments of the present disclosure.

FIGS. 5A, 5B, 5C, and 5D illustrate examples of the presentation of assessment survey questions for obtaining an assessment information set (e.g., survey results), in accordance with various embodiments of the present disclosure.

FIG. 6 illustrates an example of feedback presented from an assessment survey, in accordance with various embodiments of the present disclosure.

FIGS. 7A and 7B collectively illustrate an example method for qualifying a subject for an over-the-counter provision of a gliflozin Sodium-Glucose Cotransport 2 inhibitor pharmaceutical composition, in accordance with various embodiments of the present disclosure.

FIGS. 8A, 8B, and 8C collectively illustrate an example method for qualifying a subject for a refill (e.g., a reassessment as a result of a re-order request) of an over-the-counter provision gliflozin Sodium-Glucose Cotransport 2 inhibitor pharmaceutical composition, in accordance with various embodiments of the present disclosure.

In the figures, reference numbers refer to the same or equivalent parts of the present invention throughout the several figures of the drawing.

### DETAILED DESCRIPTION

Prediabetes is a growing health problem, in the United States and worldwide. Although prediabetes can be effectively treated using established pharmaceutical compositions, access to these drugs is hindered by to the requirement for a prescription, as many individuals do not have adequate access and/or avoid the healthcare system for a variety of reasons. Accordingly, many people are not managing their prediabetes conditions appropriately. While over-the-counter alternatives to these prescription pharmaceuticals would increase access to these compositions, thereby improving population management of diabetes around the world, patients often have difficulty self-selecting themselves for an appropriate over-the-counter medication. Because inappropriate use of these drugs can result in ineffective treatment and/or serious side-effects, better methods for selecting for, and treating patients with, other-the-counter diabetes medications are needed. The present disclosure provides, among other aspects, methods, systems, and computer readable media that solve these problems.

Reference will now be made in detail to implementations, examples of which are illustrated in the accompanying drawings. In the following detailed description of implementations, numerous specific details are set forth in order to provide a thorough understanding of the present invention. However, it will be apparent to one of ordinary skill in the art that the present invention may be practiced without these specific details.

It will also be understood that, although the terms first, second, etc. may be used herein to describe various elements, these elements should not be limited by these terms. These terms are only used to distinguish one element from another. For example, a first filter could be termed a second filter, and, similarly, a second filter could be termed a first filter, without departing from the scope of the present disclosure. The first filter and the second filter are both filters, but they are not the same filter. In some embodiments, a first computer system and a second computer system are the same computer system.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “over-the-counter” means to provide by retail purchase, subject to the constraints disclosed herein, but without a prescription or license from a physician or medical practitioner.

As used herein, the term “pharmaceutical compound” refers to any physical state of a material. Pharmaceutical compounds include but are not limited to capsules, tablets, liquids, topical formulations, and inhaled formulations.

As used herein, the term “therapeutically effective amount” or “effective amount” refers to an amount of an active pharmaceutical ingredient that is effective to achieve a desired therapeutic result (e.g., of reducing the risk of developing Type 2 diabetes and/or treatment of prediabetes).

As used herein, the term “contraindication” refers to a condition that makes a treatment, e.g., over-the-counter use of a gliflozin Sodium-Glucose Cotransport 2 inhibitor pharmaceutical composition, inadvisable. Contraindications include physical characteristics of a subject, e.g., pregnancy or liver disease, and contemporaneous drug use, e.g., gliflozin Sodium-Glucose Cotransport 2 inhibitor pharmaceutical composition use. In the present context, identification of a contraindication fires a filter of a first category class, which prevents authorizing provision of a gliflozin Sodium-Glucose Cotransport 2 inhibitor pharmaceutical composition, in accordance with some implementations of the methods, systems, and software disclosed herein.

As used herein, the term “risk factor” refers to a condition that makes a treatment, e.g., over-the-counter use of a gliflozin Sodium-Glucose Cotransport 2 inhibitor pharmaceutical composition, possibly inadvisable. Risk factors include physical characteristics of a subject, e.g., a blood pressure reading, and contemporaneous drug use, e.g., use of a blood pressure medication. In the present context, identification of a risk factor fires a filter of a second category class, which prevents authorizing provision of a gliflozin Sodium-Glucose Cotransport 2 inhibitor pharmaceutical composition without confirmation that the subject has discussed the risk factor with a medical professional, in accordance with some implementations of the methods, systems, and software disclosed herein.

As used herein, “drug interactions,” e.g., with a gliflozin Sodium-Glucose Cotransport 2 inhibitor pharmaceutical composition, include pharmacokinetic drug interactions and pharmacodynamics drug interactions. Generally, a pharmacokinetic drug interaction is an interaction between two drugs (e.g., a gliflozin Sodium-Glucose Cotransport 2 inhibitor and a second drug) that result in alterations in the absorption, transport, distribution, metabolism, and/or excretion of either drug. Generally, a pharmacokinetic drug interaction is an interaction between two drugs (e.g., a gliflozin Sodium-Glucose Cotransport 2 inhibitor and a second drug) that result in a direct change in the effect or either drug. For a more comprehensive summary of pharmacokinetic drug interactions and pharmacodynamics drug interactions, see, Cascorbi, I, Dtsch Arztebl Int., 109(33-34):546-55 (2012), the content of which is hereby incorporated by reference.

In the context of the present disclosure, classification of a condition as either a contraindication or a risk factor is specific to a particular identity and dose of a gliflozin Sodium-Glucose Cotransport 2 inhibitor pharmaceutical composition being authorized for over-the-counter use. Classification of a particular condition, e.g., contemporaneous gliflozin Sodium-Glucose Cotransport 2 inhibitor pharmaceutical composition use, may vary between different gliflozin Sodium-Glucose Cotransport 2 inhibitor pharmaceutical composition (e.g., it may be classified as a contraindication for a first gliflozin Sodium-Glucose Cotransport 2 inhibitor pharmaceutical composition, a risk factor for a second gliflozin Sodium-Glucose Cotransport 2 inhibitor pharmaceutical composition, and/or neither for a third gliflozin Sodium-Glucose Cotransport 2 inhibitor pharmaceutical composition). Likewise, a particular condition may be classified as a contraindication for use of a particular gliflozin Sodium-Glucose Cotransport 2 inhibitor pharmaceutical composition at a first over-the-counter dosage, classified as a risk factor for the same gliflozin Sodium-Glucose Cotransport 2 inhibitor pharmaceutical composition at a second (e.g., lower) over-the-counter dosage, and/or classified as neither for the same gliflozin Sodium-Glucose Cotransport 2 inhibitor pharmaceutical composition at a third (e.g., lowest) over-the-counter dosage.

As used herein, whether a subject “has developed” a condition since receiving their last provision of a gliflozin Sodium-Glucose Cotransport 2 inhibitor pharmaceutical composition refers to both conditions that are new to the subject, i.e., a condition that the subject did not have at the time they received their last provision of the gliflozin Sodium-Glucose Cotransport 2 inhibitor pharmaceutical composition, and conditions that have been newly diagnosed, regardless of whether the condition existed when the subject received their last provision of the gliflozin Sodium-Glucose Cotransport 2 inhibitor pharmaceutical composition, i.e., a condition that the subject was not aware of when they received their last provision of the gliflozin Sodium-Glucose Cotransport 2 inhibitor pharmaceutical composition.

The term “alkyl,” by itself or as part of another substituent, means, unless otherwise stated, a straight or branched chain, or cyclic hydrocarbon radical, or combination thereof, which may be fully saturated, mono- or polyunsaturated and can include di-, tri- and multivalent radicals, having the number of carbon atoms designated (e.g. C₁-C₁₀ means one to ten carbons). Examples of saturated hydrocarbon radicals include, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to optionally include those derivatives of alkyl defined in more detail below, such as “heteroalkyl.” Alkyl groups that are limited to hydrocarbon groups are termed “homoalkyl”. Exemplary alkyl groups include the monounsaturated C₉₋₁₀, oleoyl chain or the diunsaturated C_{9-10, 12-13} linoeyl chain.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “cycloalkyl” and “heterocycloalkyl,” by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, cyclopentyl, cyclohexyl, 1-cyclohexenyl, 3-cyclohexenyl, cycloheptyl, and the like. Further exemplary cycloalkyl groups include steroids, e.g., cholesterol and its derivatives. Examples of heterocycloalkyl include, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄) alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (preferably from 1 to 3 rings), which are fused together or linked covalently. The term “heteroaryl” refers to aryl substituent groups (or rings) that contain from one to four heteroatoms selected from N, O, S, Si and B, wherein the nitrogen and sulfur atoms are optionally oxidized, and the nitrogen atom(s) are optionally quaternized. An exemplary heteroaryl group is a six-membered azine, e.g., pyridinyl, diazinyl and triazinyl. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes aryl, heteroaryl and heteroarene rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy) propyl, and the like).

Each of the above terms (e.g., “alkyl,” “heteroalkyl,” “aryl, and “heteroaryl”) are meant to optionally include both substituted and unsubstituted forms of the indicated species. Exemplary substituents for these species are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: H, substituted or unsubstituted aryl, substituted or unsubstituted heteroaryl, substituted or unsubstituted heterocycloalkyl, —OR′, ═O, ═NR′, ═N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, NR C(NR′R″)═NR′″, —S(O)R′, —S(O)₂R′, —S(O)₂NR′R″, NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like). These terms encompass groups considered exemplary “alkyl group substituents,” which are components of exemplary “substituted alkyl” and “substituted heteroalkyl” moieties.

Similar to the substituents described for the alkyl radical, substituents for the aryl heteroaryl and heteroarene groups are generically referred to as “aryl group substituents.” The substituents are selected from, for example: groups attached to the heteroaryl or heteroarene nucleus through carbon or a heteroatom (e.g., P, N, O, S, Si, or B) including, without limitation, substituted or unsubstituted alkyl, substituted or unsubstituted aryl, substituted or unsubstituted heteroaryl, substituted or unsubstituted heterocycloalkyl, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, NR′ C(O)NR″R′″, —NR″C(O)₂R′, NR—C(NR′R″R′″)═NR″″, NR C(NR′R″)═NR′″, —S(O)R′, —S(O)₂R′, —S(O)₂NR′R″, NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system. Each of the above-named groups is attached to the heteroarene or heteroaryl nucleus directly or through a heteroatom (e.g., P, N, O, S, Si, or B); and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

As used herein, the term “heteroatom” includes oxygen (O), nitrogen (N), sulfur (S) and silicon (Si), boron (B) and phosphorous (P).

The symbol “R” is a general abbreviation that represents a substituent group that is selected from H, substituted or unsubstituted alkyl, substituted or unsubstituted heteroalkyl, substituted or unsubstituted aryl, substituted or unsubstituted heteroaryl, and substituted or unsubstituted heterocycloalkyl groups.

The term “salt(s)” includes salts of the compounds prepared by the neutralization of acids or bases, depending on the particular ligands or substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of base addition salts include sodium, potassium calcium, ammonium, organic amino, or magnesium salt, or a similar salt. Examples of acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids, and the like, as well as the salts derived from relatively nontoxic organic acids like acetic, propionic, isobutyric, butyric, maleic, malic, malonic, benzoic, succinic, suberic, fumaric, lactic, mandelic, phthalic, benzenesulfonic, p-tolylsulfonic, citric, tartaric, methanesulfonic, and the like. Certain specific compounds of the present invention contain both basic and acidic functionalities that allow the compounds to be converted into either base or acid addition salts. Hydrates of the salts are also included.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure or be stereoisomeric mixtures. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

In various aspects of the present disclosure a survey of a subject is conducted to obtain survey results in order to determine if the subject qualifies for an over-the-counter (OTC) gliflozin Sodium-Glucose Cotransport 2 inhibitor pharmaceutical composition for lowering blood sugar, e.g., thereby, treating prediabetes and/or delaying onset of diabetes. The survey results are used as the basis for running filters of a first category class. If the triggering conditions of any of the filters in the first category class are fired, the subject does not qualify for the OTC gliflozin Sodium-Glucose Cotransport 2 inhibitor pharmaceutical composition. The survey results are also used as the basis for running filters of a second category class. If the triggering conditions of any of the filters in the second category class are fired, the subject is provided with warning messages associated with the respective filters of the second category class that have been fired. If none of the filters in the first category class are fired and the subject successfully addresses the warning messages associated with the respective filters of the second category class that have been fired a fulfillment process is initiated for OTC access to a provision of the gliflozin Sodium-Glucose Cotransport 2 inhibitor pharmaceutical composition.

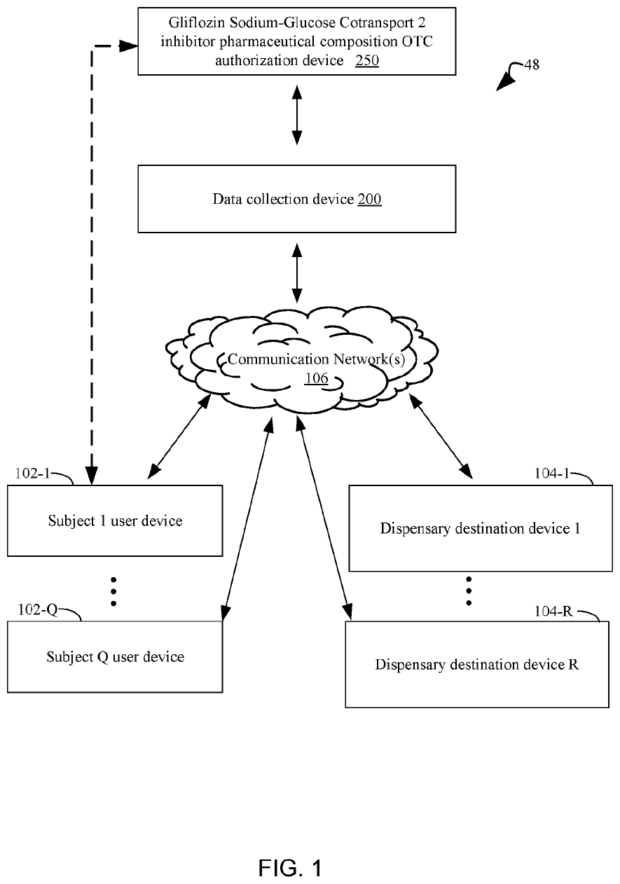

FIG. 1 illustrates an example of an integrated system 48 for conducting one or more surveys of subjects in order to qualifying the subjects for OTC delivery of a gliflozin Sodium-Glucose Cotransport 2 inhibitor pharmaceutical composition. The integrated system 48 includes one or more connected user devices 102. The user devices 102 are configured for entering survey data and making requests for the gliflozin Sodium-Glucose Cotransport 2 inhibitor pharmaceutical composition. The system 48 also includes one or more dispensary destination devices 104 that are configured to receive instructions in order to provide the gliflozin Sodium-Glucose Cotransport 2 inhibitor pharmaceutical composition to qualifying subjects. Furthermore, the system 48 includes a gliflozin Sodium-Glucose Cotransport 2 inhibitor pharmaceutical composition over-the-counter (OTC) dispensing device 250 and one or more data collection devices 200 that are configured for collecting subject data.

Throughout the present disclosure, the data collection device 200 and the gliflozin Sodium-Glucose Cotransport 2 inhibitor pharmaceutical composition OTC dispensing device 250 will be referenced as separate devices solely for purposes of clarity. That is, the disclosed functionality of the data collection device 200 and the disclosed functionality of the gliflozin Sodium-Glucose Cotransport 2 inhibitor pharmaceutical composition OTC dispensing device 250 are contained in separate devices as illustrated in FIG. 1. However, it will be appreciated that, in some embodiments, the disclosed functionality of the data collection device 200 and the disclosed functionality of the gliflozin Sodium-Glucose Cotransport 2 inhibitor pharmaceutical composition OTC dispensing device 250 are contained in a single device.

With the integrated system 48, survey results (e.g., the assessment information set and/or the reassessment information set) from the subjects are run against a first set of assessment filters (e.g., filter 216-1, filter 216-2, filter 216-3, etc.) When a filter in the first set of filters (e.g., the first set of assessment filters and/or the first set of reassessment filters) is fired for a respective subject, the respective subject is deemed not qualified for the gliflozin Sodium-Glucose Cotransport 2 inhibitor pharmaceutical composition. The survey results are also run against a second set of filters (e.g., filter 222-1, etc.) When a respective filter in the second set of filters (e.g., the second set of assessment filters and/or optional second set of reassessment filters) is fired for a respective subject, the respective subject is provided with a warning (e.g., filter warning 226) associated with the respective filter. In some embodiments the survey results are run against the first set of assessment filters and the second set of assessment filters concurrently (and/or against the first set of reassessment filters and the optional second set of reassessment filters concurrently). In some embodiments the survey results are run against the first set of assessment filters and then against the second set of assessment filters sequentially (and/or against the first set of reassessment filters and then against the optional second set of reassessment filters sequentially). The method enabled by the integrated system 48 proceeds to a fulfillment process when no filter in the first plurality of filters fires and the subject has acknowledged, or otherwise successfully addressed, each warning associated with each filter in the second plurality of filters that fired. As part of the fulfillment process, authorization is granted to the subject for a provision of the gliflozin Sodium-Glucose Cotransport 2 inhibitor pharmaceutical composition, and the authorization is stored (e.g., in a user profile 234 associated with the subject to receive the drug). In some embodiments, a drug facts label (e.g., drug facts label 230) for the gliflozin Sodium-Glucose Cotransport 2 inhibitor pharmaceutical composition is communicated to the qualifying subject, and authorization occurs upon subject confirmation that the label has been read.

Referring to FIG. 1, the gliflozin Sodium-Glucose Cotransport 2 inhibitor pharmaceutical composition OTC dispensing device 250 authorizes a subject for over-the-counter delivery of a gliflozin Sodium-Glucose Cotransport 2 inhibitor pharmaceutical composition to lower blood sugar, thereby treating prediabetes and/or delaying onset of diabetes. To accomplish this, the data collection device 200, which is in electrical communication with the gliflozin Sodium-Glucose Cotransport 2 inhibitor pharmaceutical composition OTC dispensing device 250, receives survey results originating from one or more user devices 102 associated with corresponding subjects. In some embodiments, the data collection device 200 receives such survey results directly from the user devices 102. For instance, in some embodiments the data collection device 200 receives this data wirelessly through radio-frequency signals. In some embodiments, such signals are in accordance with an 802.11 (Wi-Fi), Bluetooth, or ZigBee standard. In some embodiments, the data collection device 200 receives such data directly, analyzes the data, and passes the analyzed data to the gliflozin Sodium-Glucose Cotransport 2 inhibitor pharmaceutical composition OTC dispensing device 250.

In some embodiments, the data collection device 200 and/or the gliflozin Sodium-Glucose Cotransport 2 inhibitor pharmaceutical composition OTC dispensing device 250 is not proximate to the subject and/or does not have wireless capabilities or such wireless capabilities are not used for the purpose of acquiring survey results. In such embodiments, a communication network 106 may be used to survey questions (e.g., survey questions 208, 212) from the gliflozin Sodium-Glucose Cotransport 2 inhibitor pharmaceutical composition OTC dispensing device 250 to user devices 102 and the answers to such survey questions from the user devices 102 to the data collection device 200 and/or the gliflozin Sodium-Glucose Cotransport 2 inhibitor pharmaceutical composition OTC dispensing device 250. Further, in some embodiments the communication network 106 is used to communicate authorization to dispense the gliflozin Sodium-Glucose Cotransport 2 inhibitor pharmaceutical composition survey questions from the gliflozin Sodium-Glucose Cotransport 2 inhibitor pharmaceutical composition OTC dispensing device 250 to dispensary destination devices 104.

Examples of networks 106 include, but are not limited to, the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11 ac, IEEE 802.11ax, IEEE 802.11b, IEEE 802.11g and/or IEEE 802.11n), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of the present disclosure.

Of course, other topologies of the system 48 are possible. For instance, rather than relying on a communications network 106, the one or more user devices 102 and the one or more dispensary destination devices 104 may communicate directly to the data collection device 200 and/or the gliflozin Sodium-Glucose Cotransport 2 inhibitor pharmaceutical composition OTC dispensing device 250. Further, the data collection device 200 and/or the gliflozin Sodium-Glucose Cotransport 2 inhibitor pharmaceutical composition OTC dispensing device 250 may constitute a portable electronic device, a server computer, or in fact constitute several computers that are linked together in a network, be a virtual machine in a cloud computing context, be a container in a cloud computer context, or a combination thereof. As such, the exemplary topology shown in FIG. 1 merely serves to describe the features of an embodiment of the present disclosure in a manner that will be readily understood to one of skill in the art.

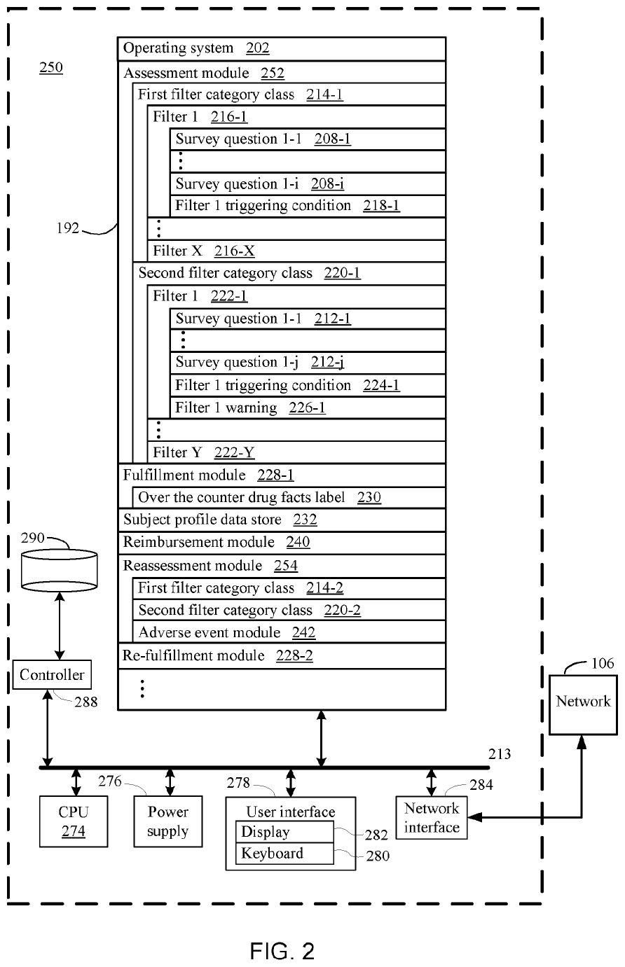

Turning to FIG. 2 with the foregoing in mind, an exemplary gliflozin Sodium-Glucose Cotransport 2 inhibitor pharmaceutical composition OTC dispensing device 250 configured for determining whether a subject is qualified for OTC provision of a gliflozin Sodium-Glucose Cotransport 2 inhibitor pharmaceutical composition is depicted. Referring to FIG. 2, in typical embodiments, the gliflozin Sodium-Glucose Cotransport 2 inhibitor pharmaceutical composition OTC dispensing device 250 includes one or more computers. For purposes of illustration in FIG. 2, the gliflozin Sodium-Glucose Cotransport 2 inhibitor pharmaceutical composition OTC dispensing device 250 is represented as a single computer that includes all of the functionality for qualifying a human subject for over-the-counter delivery of a gliflozin Sodium-Glucose Cotransport 2 inhibitor pharmaceutical composition to lower blood sugar. However, the present disclosure is not limited thereto. In some embodiments, the functionality for qualifying a human subject for over-the-counter delivery of a gliflozin Sodium-Glucose Cotransport 2 inhibitor pharmaceutical composition to lower blood sugar is spread across any number of networked computers and/or resides on each of several networked computers, is hosted on one or more virtual machines at a remote location accessible across the communications network 106, and/or is hosted on one or more containers at a remote location accessible across the communications network 106. One of skill in the art will appreciate that any of a wide array of different computer topologies are used for the application and all such topologies are within the scope of the present disclosure.

The gliflozin Sodium-Glucose Cotransport 2 inhibitor pharmaceutical composition OTC dispensing device 250 of FIG. 2 is configured to conduct an assessment survey (e.g., using assessment module 252 to perform an initial qualification of the subject for provision of a gliflozin Sodium-Glucose Cotransport 2 inhibitor pharmaceutical composition) and/or a reassessment survey (e.g., using reassessment module 254 to perform a re-qualification of the subject for provision of a gliflozin Sodium-Glucose Cotransport 2 inhibitor pharmaceutical composition). The assessment survey (e.g., the first survey) includes a variety of questions 208, 212 associated with filters 216, 222 within a first set of assessment filters 214 (e.g., a set of filters of the first filter category class) and a second set of assessment filters 220 (e.g., a set of filters in the second filter category class), respectively. Answers to the questions in the assessment survey received by the device are run against filters of a first category class 214 and filters of a second category class 220 within the first and second sets of assessment filters 216, 222, respectively. Similarly, a reassessment survey (e.g., a second survey) also includes a variety of questions 258 associated with filters 217, 223 within a set of filters of a first category class 215 (e.g., a first set of reassessment filters) and an optional set of filters of a second category class 221 (e.g., an optional second set of reassessment filters), respectively. Answers to the questions in the reassessment survey received by the device are run against filters of a first category class 217 and optionally filters of a second category class 221, e.g., within the first and optional second sets of filters, respectively. Filters 216/217 of the first filter category class 214/215 are configured to terminate the qualification process when fired. Filters 222/223 of the second filter category class 220/221 are configured to provide the subject with a warning associated with a corresponding survey question. In other words, the device of FIG. 2 is configured to accumulate results from a survey (e.g., survey questions 208 and survey questions 212) and run the results against corresponding filters (e.g., filters 216/217 and filters 222/223, respectively) in order to determine if a subject is qualified for OTC delivery of a gliflozin Sodium-Glucose Cotransport 2 inhibitor pharmaceutical composition.

In the present disclosure, a plurality of filters refers to a series, or set, or filters in either the first set of assessment filters or the second set of assessment filters (or, for the reassessment, the first set of reassessment filters or the optional second plurality of reassessment filters). For instance, in some embodiments, a plurality of filters of the first set of assessment filters 214 can comprise any subset of filters 216 of the first set of assessment filters. As an example, in some embodiments a plurality of filters of the first set of assessment filters includes filters 216-1, 216-2, 216-3, . . . , 216-X, or any combination thereof. Similarly, a plurality of filters of the second set of assessment filters 220-1 can comprise any set of filters 222 of the second set of assessment filters.

Continuing to refer to FIG. 2, in some embodiments, the dispensing device 250 includes one or more processing units (CPU's) 274, a network or other communications interface 284, a memory 192 (e.g., random access memory), one or more magnetic disk storage and/or persistent devices 290 optionally accessed by one or more controllers 288, one or more communication busses 213 for interconnecting the aforementioned components, a user interface 278, the user interface 278 including a display 282 and input 280 (e.g., keyboard, keypad, touch screen), and a power supply 276 for powering the aforementioned components. In some embodiments, data in memory 192 is seamlessly shared with non-volatile memory 290 using known computing techniques such as caching. In some embodiments, memory 192 and/or memory 290 includes mass storage that is remotely located with respect to the central processing unit(s) 274. In other words, some data stored in memory 192 and/or memory 290 may in fact be hosted on computers that are external to the gliflozin Sodium-Glucose Cotransport 2 inhibitor pharmaceutical composition OTC dispensing device 250 but that can be electronically accessed by the gliflozin Sodium-Glucose Cotransport 2 inhibitor pharmaceutical composition OTC dispensing device 250 over an Internet, intranet, or other form of network or electronic cable (illustrated as element 106 in FIG. 2) using network interface 284.

In some embodiments, the memory 192 of the gliflozin Sodium-Glucose Cotransport 2 inhibitor pharmaceutical composition OTC dispensing device 250 stores one or more of:
an operating system 202 that includes procedures for handling various basic system services;
an assessment module 252 for qualifying a subject for an initial over-the-counter provision of a gliflozin Sodium-Glucose Cotransport 2 inhibitor pharmaceutical composition, by communicating survey questions, obtaining results therefrom, and applying the results to qualifying filters, the assessment module including:
   a first filter category class 214, including filters 216 (e.g., a first set of assessment filters), each respective filter 216 in the first filter category class 214 associated with one or more survey questions 208 and one or more triggering conditions 218;
   a second filter category class 220, including filters 222 (e.g., a second set of assessment filters), each respective filter 222 in the second filter category class 220 associated with one or more survey questions 208, triggering conditions 224, and warnings 226;
a reassessment module 254 for qualifying a subject for a subsequent over-the-counter provision of a gliflozin Sodium-Glucose Cotransport 2 inhibitor pharmaceutical composition by communicating survey questions, obtaining a reassessment information set therefrom, and applying the reassessment information set to qualifying filters, the assessment module including:
   a first filter category class 215, including filters 217 (e.g., a first set of reassessment filters), each respective filter 217 in the first filter category class 215 associated with one or more survey questions 208 and one or more triggering conditions 218;
   an optional second filter category class 221, including filters 223 (e.g., a second set of reassessment filters), each respective filter 223 in the second filter category class 221 associated with one or more survey questions 208, triggering conditions 224, and warnings 226;
a fulfillment module 228-1 for executing a fulfillment process when no filter 216 in either the first filter category class 214 has been fired for a subject and the subject has acknowledged each warning 226 associated with each filter 222 in the second filter category class 220 that was fired as a result of answers by the subject to the survey questions 208, where, in some embodiments, the fulfillment process includes communicating an over-the-counter drug facts label 230 for the gliflozin Sodium-Glucose Cotransport 2 inhibitor pharmaceutical composition to the subject and receiving confirmation from the subject that the over-the-counter drug facts label has been received and read;
a re-fulfillment module 228-2 for executing a re-fulfillment process when no filter 217 in the first filter category class 215 has been fired for a subject and the subject has acknowledged each warning 226 associated with each filter 223 in the optional second filter category class 221 that was fired as a result of answers by the subject to the survey questions 212, where the re-fulfillment process includes, in some embodiments, communicating an over-the-counter drug facts label 230 for the gliflozin Sodium-Glucose Cotransport 2 inhibitor pharmaceutical composition to the subject and receiving confirmation from the subject that the over-the-counter drug facts label has been received and read;
a subject profile data store 232 comprising a user profile 234 for each of a plurality of subjects, each respective user profile 234 including information (e.g., shipping information, billing information, biometric information, etc.) about a corresponding subject in the plurality of subjects, an initial authorization date and optional destination 236, and any reauthorization date and the optional destination 238 for the gliflozin Sodium-Glucose Cotransport 2 inhibitor pharmaceutical composition made by the corresponding subject using the gliflozin Sodium-Glucose Cotransport 2 inhibitor pharmaceutical composition OTC dispensing device 250;
an adverse event module 242 for identifying and aggregating records of adverse events associated with a plurality of subjects, e.g., corresponding to the firing of a filter 217 in the first filter category class 215 during a re-fulfillment process; and
an optional reimbursement module 240 for determining eligibility and/or communicating an insurance claim associated with acquisition of the gliflozin Sodium-Glucose Cotransport 2 inhibitor pharmaceutical composition, e.g., based on insurance information stored in a respective user profile 234.

In some embodiments, the assessment module 252, reassessment module 254, and/or fulfillment module 228 are accessible within any browser (e.g., phone, tablet, laptop/desktop, or smartwatch). In some embodiments, the assessment module 252, reassessment module 254, and/or fulfillment module 228 run on native device frameworks, and are available for download onto a user device 102 running an operating system 202 such as Android, iOS, or WINDOWS.

In some implementations, one or more of the above identified data elements or modules (e.g., assessment module 252, fulfillment module 228-1, etc.) of the gliflozin Sodium-Glucose Cotransport 2 inhibitor pharmaceutical composition OTC dispensing device 250 for qualifying a human subject for over-the-counter delivery of a gliflozin Sodium-Glucose Cotransport 2 inhibitor pharmaceutical composition to lower blood sugar are stored in one or more of the previously described memory devices, and correspond to a set of instructions for performing a function described above. The above-identified data, modules or programs (e.g., sets of instructions) need not be implemented as separate software programs, procedures or modules, and thus various subsets of these modules may be combined or otherwise re-arranged in various implementations. In some implementations, the memory 192 and/or 290 optionally stores a subset of the modules and data structures identified above. Furthermore, in some embodiments the memory 192 and/or 290 stores additional modules and data structures not described above.

In some embodiments, a gliflozin Sodium-Glucose Cotransport 2 inhibitor pharmaceutical composition OTC dispensing device 250 for qualifying a human subject for over-the-counter provision of a gliflozin Sodium-Glucose Cotransport 2 inhibitor pharmaceutical composition to lower blood sugar is a smart phone (e.g., an iPhone, Blackberry, etc.), a laptop, a tablet computer, a desktop computer, a smart watch, or another form of electronic device (e.g., a gaming console). In some embodiments, the gliflozin Sodium-Glucose Cotransport 2 inhibitor pharmaceutical composition OTC dispensing device 250 is not mobile. In some embodiments, the gliflozin Sodium-Glucose Cotransport 2 inhibitor pharmaceutical composition OTC dispensing device 250 is mobile.

In some embodiments, the gliflozin Sodium-Glucose Cotransport 2 inhibitor pharmaceutical composition OTC dispensing device 250 is not a smart phone but rather is a tablet computer, desktop computer, emergency vehicle computer, or other form or wired or wireless networked device. In the interest of brevity and clarity, only a few of the possible components of the gliflozin Sodium-Glucose Cotransport 2 inhibitor pharmaceutical composition OTC dispensing device 250 are shown in FIG. 2 in order to better emphasize the additional software modules that are installed on the gliflozin Sodium-Glucose Cotransport 2 inhibitor pharmaceutical composition OTC dispensing device 250.

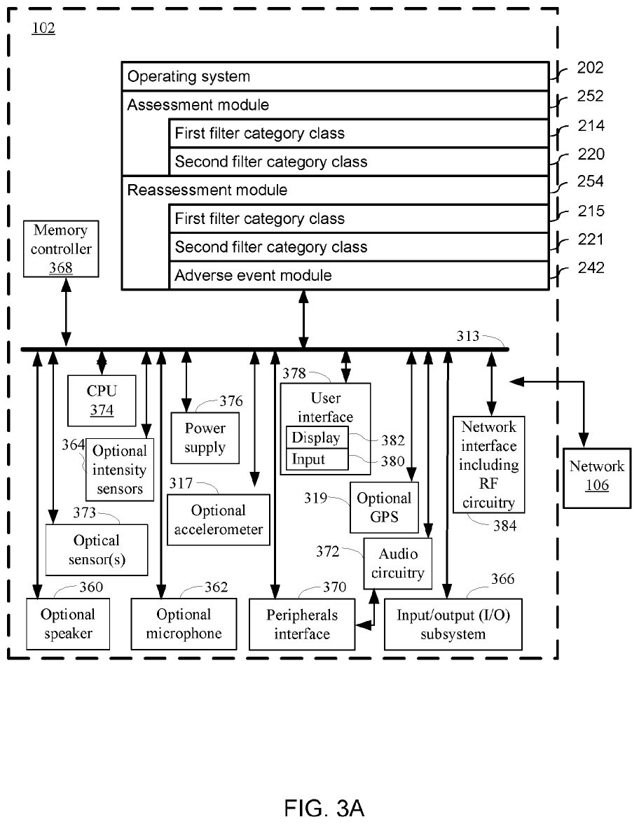

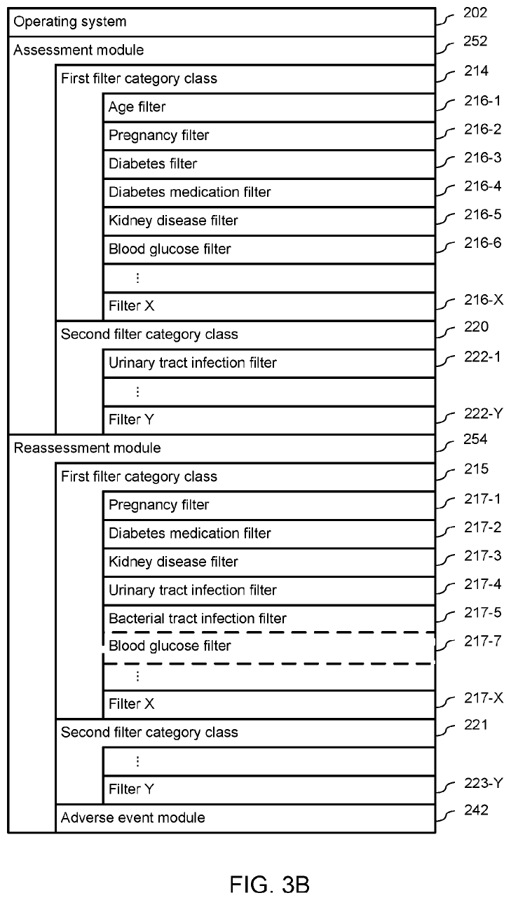

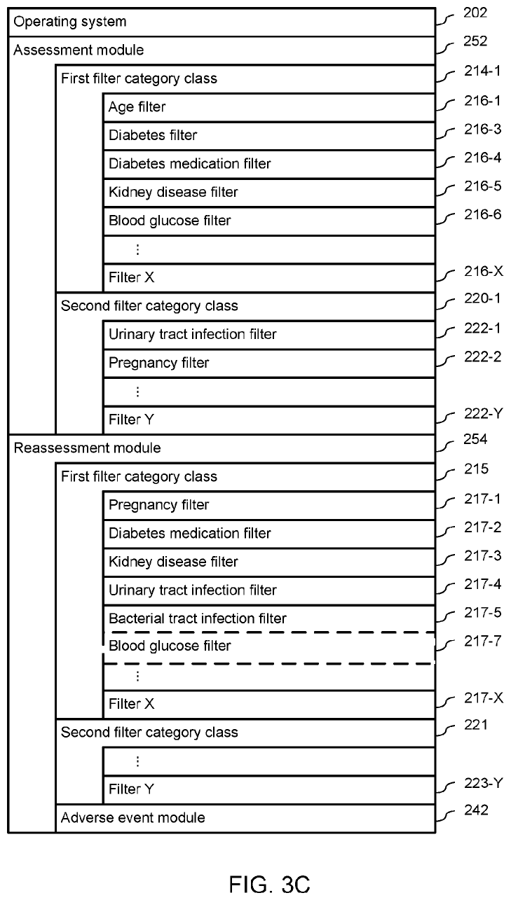

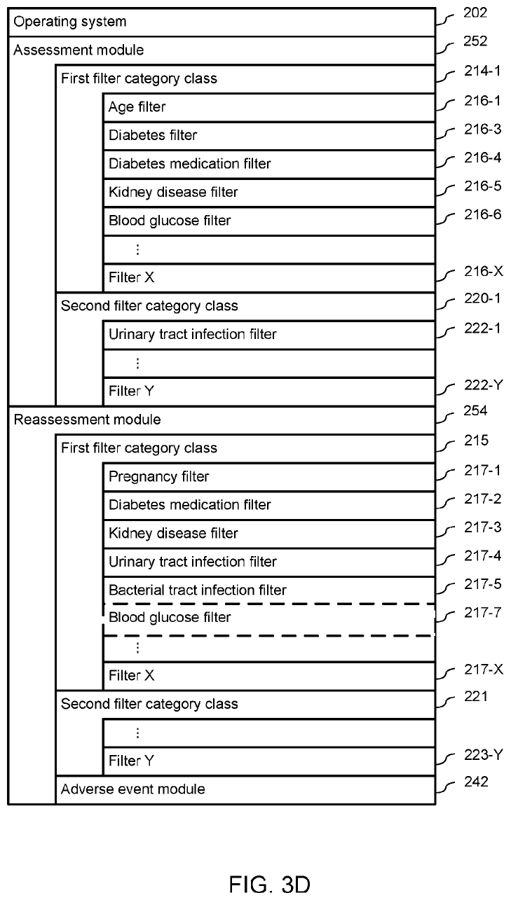

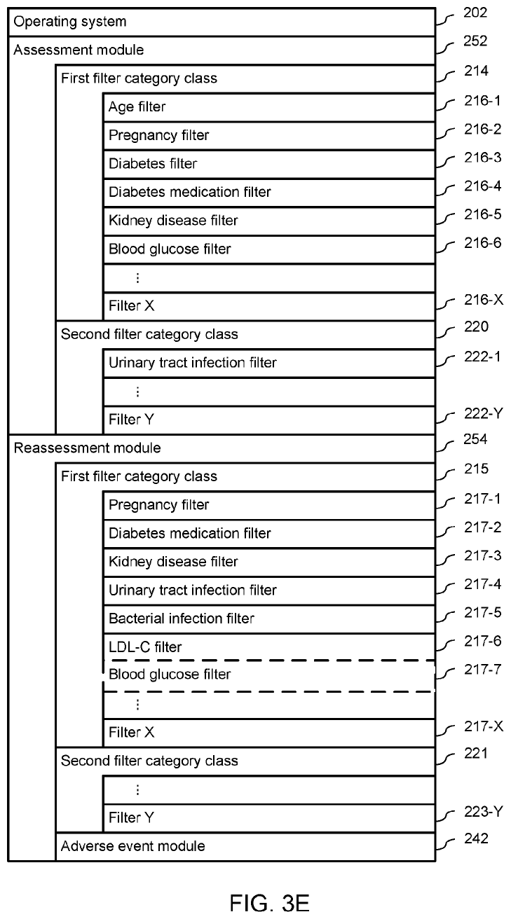

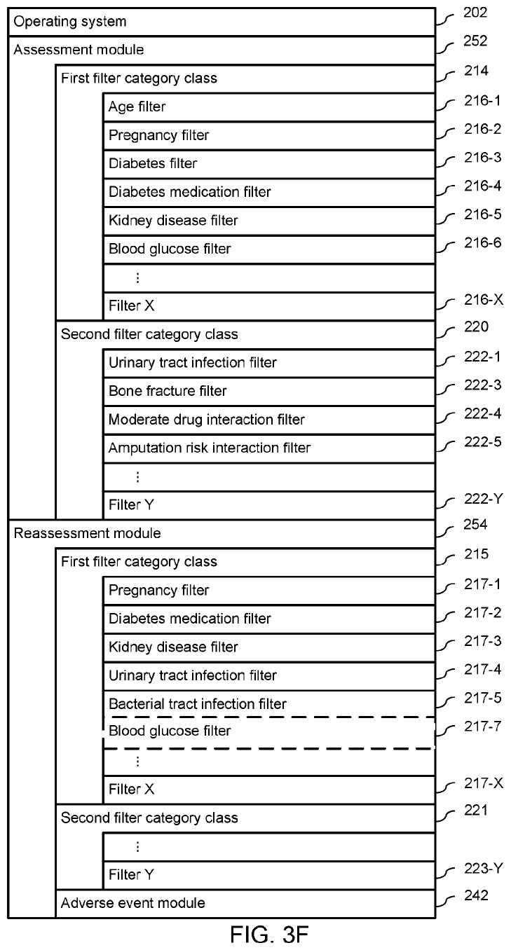

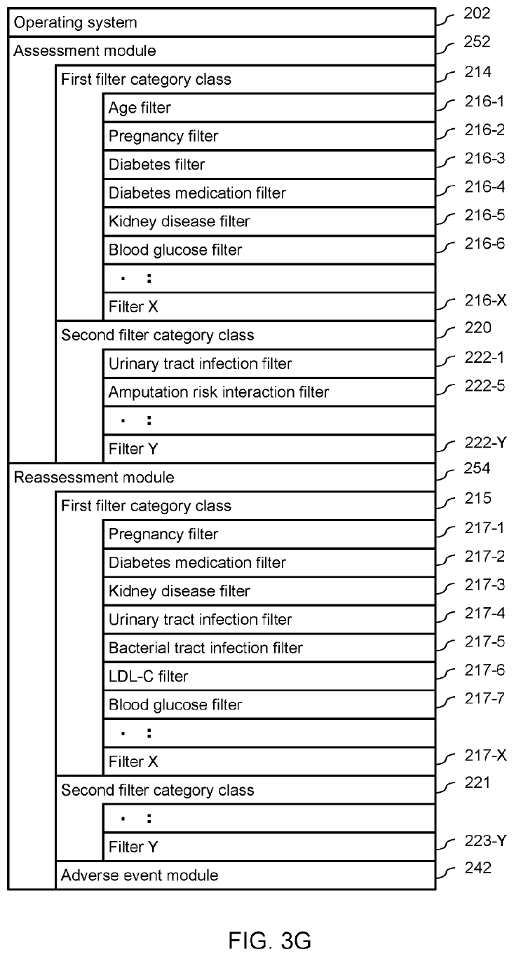

FIG. 3 provides a description of a user device 102 that can be used with the present disclosure. The user device 102 illustrated in FIG. 3 has one or more processing units (CPU's) 374, peripherals interface 370, memory controller 368, a network or other communications interface 384, a memory 392 (e.g., random access memory), a user interface 378, the user interface 378 including a display 382 and input 380 (e.g., keyboard, keypad, touch screen), an optional accelerometer 317, an optional GPS 319, optional audio circuitry 372, an optional speaker 360, an optional microphone 362, one or more optional intensity sensors 364 for detecting intensity of contacts on the user device 102 (e.g., a touch-sensitive surface such as a touch-sensitive display system 382 of the user device 102), an optional input/output (I/O) subsystem 366, one or more optional optical sensors 373, one or more communication busses 313 for interconnecting the aforementioned components, and a power supply 376 for powering the aforementioned components.

In some embodiments, the input 380 is a touch-sensitive display, such as a touch-sensitive surface. In some embodiments, the user interface 378 includes one or more soft keyboard embodiments. The soft keyboard embodiments may include standard (e.g., QWERTY) and/or non-standard configurations of symbols on the displayed icons.

The user device 102 illustrated in FIG. 3 optionally includes, in addition to accelerometer(s) 317, a magnetometer (not shown) and a GPS 319 (or GLONASS or other global navigation system) receiver for obtaining information concerning the location and orientation (e.g., portrait or landscape) of the user device 102 and/or for determining an amount of physical exertion by the subject.

It should be appreciated that the user device 102 illustrated in FIG. 3 is only one example of a multifunction device that may be used for performing a survey (e.g., assessment survey 206) in order to qualify for over-the-counter delivery of a gliflozin Sodium-Glucose Cotransport 2 inhibitor pharmaceutical composition to lower blood sugar, and that the user device 102 optionally has more or fewer components than shown, optionally combines two or more components, or optionally has a different configuration or arrangement of the components. The various components shown in FIG. 3 are implemented in hardware, software, firmware, or a combination thereof, including one or more signal processing and/or application specific integrated circuits.

Memory 392 of the user device 102 illustrated in FIG. 3A optionally includes high-speed random access memory and optionally also includes non-volatile memory, such as one or more magnetic disk storage devices, flash memory devices, or other non-volatile solid-state memory devices. Access to memory 392 by other components of the user device 102, such as CPU(s) 374 is, optionally, controlled by the memory controller 368. In some embodiments, the memory 392 of the user device 102 illustrated in FIG. 3A optionally includes:
an operating system 302 that includes procedures for handling various basic system services;
an assessment module 252 described above in conjunction with the gliflozin Sodium-Glucose Cotransport 2 inhibitor pharmaceutical composition OTC dispensing device 250, the assessment module 252-1 comprising:
   a first set of assessment filters 214, described above in conjunction with the gliflozin Sodium-Glucose Cotransport 2 inhibitor pharmaceutical composition OTC dispensing device 250, comprising one or more filters 216, e.g., as shown in FIGS. 3B-3D, and
   a second set of assessment filters 220, described above in conjunction with the gliflozin Sodium-Glucose Cotransport 2 inhibitor pharmaceutical composition OTC dispensing device 250, comprising one or more filters 222, e.g., as shown in FIGS. 3B-3D; and
an reassessment module 254 described above in conjunction with the gliflozin Sodium-Glucose Cotransport 2 inhibitor pharmaceutical composition OTC dispensing device 250, the reassessment module 252 comprising:
   a first set of reassessment filters 215, described above in conjunction with the gliflozin Sodium-Glucose Cotransport 2 inhibitor pharmaceutical composition OTC dispensing device 250, comprising one or more filters 217, e.g., as shown in FIGS. 3B-3D;
   an optional second set of reassessment filters 221, described above in conjunction with the gliflozin Sodium-Glucose Cotransport 2 inhibitor pharmaceutical composition OTC dispensing device 250, comprising one or more filters 223, e.g., as shown in FIGS. 3B-3D.

FIGS. 3B-3D show example implementations of the assessment module 252 and reassessment module 254 described above, in accordance with various embodiments of the present disclosure.

For example, as illustrated in FIG. 3B, in some embodiments an assessment module 252 includes a first set of assessment filters 214 comprising an age filter 216-1, a pregnancy filter 216-2, a diabetes filter 216-3, a diabetes medication filter 216-4, a kidney disease filter 216-5, and a blood glucose filter 216-6, and a second set of assessment filters 220 comprising a urinary tract infection filter 222-1. Similarly, as also illustrated in FIG. 3B, in some embodiments a reassessment module 254 includes a first set of reassessment filters 215 comprising a pregnancy filter 217-1, a diabetes medication filter 217-2, a kidney disease filter 217-3, a urinary tract infection filter 217-4, and a bacterial tract infection filter 217-4.

Likewise, as illustrated in FIG. 3C, in some embodiments an assessment module 252 includes a first set of assessment filters 214 comprising an age filter 216-1, a diabetes filter 216-3, a diabetes medication filter 216-4, a kidney disease filter 216-5, and a blood glucose filter 216-6, and a second set of assessment filters 220 comprising a urinary tract infection filter 222-1 and a pregnancy filter 222-2. Similarly, as also illustrated in FIG. 3C, in some embodiments a reassessment module 254 includes a first set of reassessment filters 215 comprising a pregnancy filter 217-1, a diabetes medication filter 217-2, a kidney disease filter 217-3, a urinary tract infection filter 217-4, and a bacterial tract infection filter 217-4.

Likewise, as illustrated in FIG. 3D, in some embodiments an assessment module 252 includes a first set of assessment filters 214 comprising an age filter 216-1, a diabetes filter 216-3, a diabetes medication filter 216-4, a kidney disease filter 216-5, and a blood glucose filter 216-6, and a second set of assessment filters 220 comprising a urinary tract infection filter 222-1. Similarly, as also illustrated in FIG. 3D, in some embodiments a reassessment module 254 includes a first set of reassessment filters 215 comprising a pregnancy filter 217-1, a diabetes medication filter 217-2, a kidney disease filter 217-3, a urinary tract infection filter 217-4, and a bacterial tract infection filter 217-4.

In some embodiments, the optional accelerometer 317, optional GPS 319, and/or magnetometer (not shown) of the user device 102 or such components are used to recommend to qualifying subjects one or more suitable destinations for access to the gliflozin Sodium-Glucose Cotransport 2 inhibitor pharmaceutical composition over-the-counter. In some embodiments, the GPS 319 is used to determine if a subject is geographically restricted for OTC access of the gliflozin Sodium-Glucose Cotransport 2 inhibitor pharmaceutical composition. Geographical restrictions include but are not limited to a subject residing outside of delivery or shipping regions, marketing restrictions, and/or government regulations.

The peripherals interface 370 can be used to couple input and output peripherals of the device to CPU(s) 374 and memory 392. The one or more processors 374 run or execute various software programs and/or sets of instructions stored in memory 392, such as the survey module 204, to perform various functions for the user device 102 and to process data.

In some embodiments, the peripherals interface 370, CPU(s) 374, and memory controller 368 are, optionally, implemented on a single chip. In some other embodiments, they are implemented on separate chips.

RF (radio frequency) circuitry of network interface 384 receives and sends RF signals, also called electromagnetic signals. In some embodiments, the survey module 204, survey questions 208/212, answers to survey questions 208/212, and/or the over-the-counter drug facts label 230 are communicated to the subject device 102 using this RF circuitry. In some embodiments, the RF circuitry 384 converts electrical signals to/from electromagnetic signals and communicates with communications networks and other communications devices and/or the data collection device 200 and/or the gliflozin Sodium-Glucose Cotransport 2 inhibitor pharmaceutical composition OTC dispensing device 250 via the electromagnetic signals. The RF circuitry 384 optionally includes well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth. RF circuitry 384 optionally communicates with the communication network 106. In some embodiments, the circuitry 384 does not include RF circuitry and, in fact, is connected to the network 106 through one or more hard wires (e.g., an optical cable, a coaxial cable, or the like).

In some embodiments, the audio circuitry 372, the optional speaker 360, and the optional microphone 362 provide an audio interface between the subject and the user device 102. The audio circuitry 372 receives audio data from the peripherals interface 370, converts the audio data to electrical signals, and transmits the electrical signals to the speaker 360. The speaker 360 converts the electrical signals to human-audible sound waves. In some embodiments, the speaker 260 converts the electrical signals to human-inaudible sound waves. The audio circuitry 372 also receives electrical signals converted by the microphone 362 from sound waves. The audio circuitry 372 converts the electrical signal to audio data and transmits the audio data to peripherals interface 370 for processing. Audio data is, optionally, retrieved from and/or transmitted to the memory 392 and/or the RF circuitry 384 by the peripherals interface 370.

In some embodiments, the power supply 376 optionally includes a power management system, one or more power sources (e.g., battery, alternating current (AC)), a recharging system, a power failure detection circuit, a power converter or inverter, a power status indicator (e.g., a light-emitting diode (LED)) and any other components associated with the generation, management and distribution of power in portable devices.

In some embodiments, the user device 102 optionally also includes one or more optical sensors 373. The optical sensor(s) 373 optionally include charge-coupled device (CCD) or complementary metal-oxide semiconductor (CMOS) phototransistors. The optical sensor(s) 373 receive light from the environment, projected through one or more lens, and converts the light to data representing an image. The optical sensor(s) 373 optionally capture still images and/or video. In some embodiments, an optical sensor is located on the back of the user device 102, opposite the display 382 on the front of the user device 102, so that the input 380 is enabled for use as a viewfinder for still and/or video image acquisition. In some embodiments, another optical sensor 373 is located on the front of the user device 102 so that the subject's image is obtained, e.g., to verify the health, condition, or identity of the subject as part of qualifying the subject for over-the-counter delivery of a gliflozin Sodium-Glucose Cotransport 2 inhibitor pharmaceutical composition to lower blood sugar, to help diagnose a subject's condition remotely, or to acquire visual physiological measurements of the subject, etc.

As illustrated in FIGS. 3A-3D, the user device 102 preferably includes an operating system 302 that includes procedures for handling various basic system services. The operating system 302 (e.g., iOS, DARWIN, RTXC, LINUX, UNIX, OS X, WINDOWS, or an embedded operating system such as VxWorks) includes various software components and/or drivers for controlling and managing general system tasks (e.g., memory management, storage device control, power management, etc.) and facilitates communication between various hardware and software components.

In some embodiments the user device 102 is a smart phone or a smart watch. In other embodiments, the user device 102 is not a smart phone or a smart watch but rather is a tablet computer, a desktop computer, an emergency vehicle computer, or other form or wired or wireless networked device. In the interest of brevity and clarity, only a few of the possible components of the user device 102 are shown in FIGS. 3A-3D in order to better emphasize the additional software modules that are installed on the user device 102.

While the system 48 disclosed in FIG. 1 can work standalone, in some embodiments it can also be linked with electronic medical record systems to exchange information in any way.

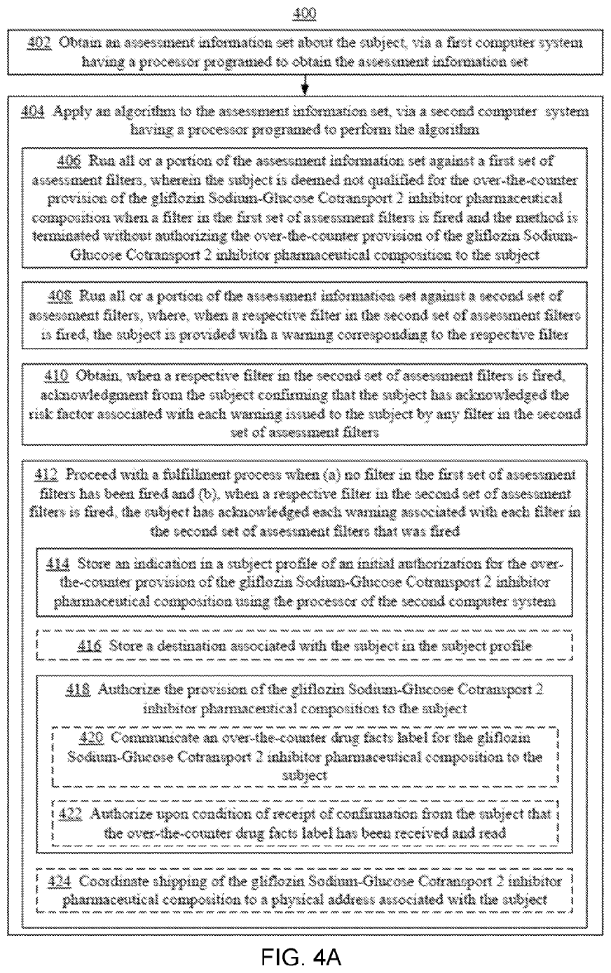

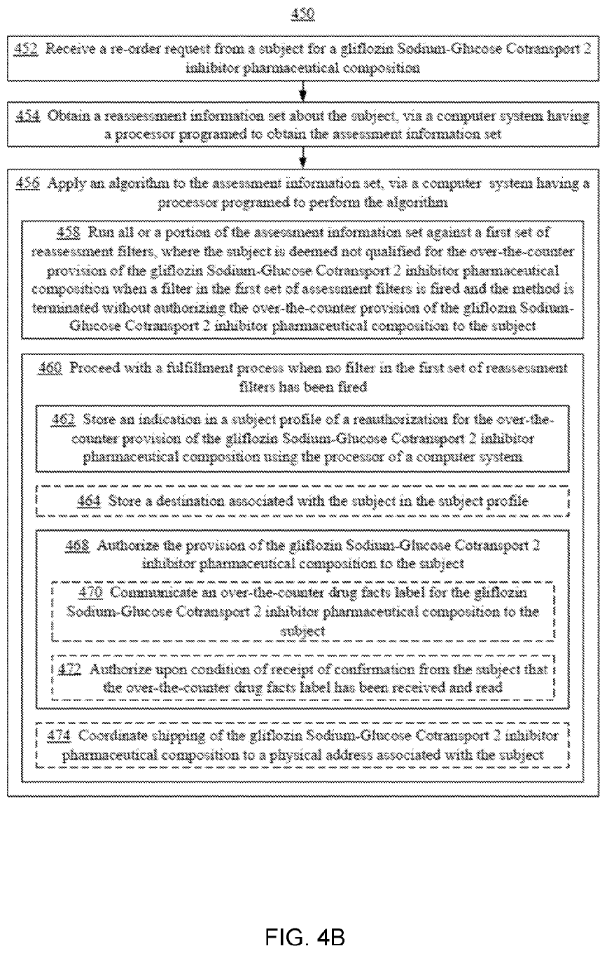

Now that details of a system 48 for qualifying a human subject for over-the-counter provision of a gliflozin Sodium-Glucose Cotransport 2 inhibitor pharmaceutical composition to lower blood sugar, e.g., thereby treating prediabetes and/or delaying onset of diabetes, have been disclosed, details about methods 400 and 450, including processes and features to be performed by the system, in accordance with various embodiments of the present disclosure, are disclosed with reference to FIGS. 4A and 4B, respectively. In some embodiments, such processes and features of the system are carried out by the assessment module 252, reassessment module 254, fulfillment module 228-1, and/or re-fulfillment module 228-2 illustrated in FIGS. 2 and 3. In some embodiments, the assessment module 252, reassessment module 254, fulfillment module 228-1, and/or re-fulfillment module 228-1 are a single software module. In the flow chart, elements in dashed boxes are optional.

### Initial Assessment for Authorizing a Human Subject for an Over-the-Counter (OTC) Provision of a Gliflozin Sodium-Glucose Cotransport 2 Inhibitor Pharmaceutical Composition

One goal of the present disclosure is to provide methods and systems for qualifying subjects for an initial over-the-counter provision of a gliflozin Sodium-Glucose Cotransport 2 inhibitor pharmaceutical composition in a more effective manner than conventional subject self-selection, in which a subject must decide for themselves whether they are a suitable candidate for a particular pharmaceutical agent, e.g., while in an isle of a pharmacy. As described above, studies have shown that subjects tend not to carefully read, if at all, drug labels at a pharmacy. And, when they do read the labels, subjects often do not properly comprehend the indicia for proper use and/or warnings associated with certain personal characteristics. This is particularly important when considering whether to take gliflozin Sodium-Glucose Cotransport 2 inhibitor pharmaceutical compositions, which are associated with severe adverse reactions when not properly taken.

Advantageously, the present disclosure provides methods and systems that better facilitate qualification of subjects for an initial provision of a gliflozin Sodium-Glucose Cotransport 2 inhibitor pharmaceutical composition. For instance, method 400 as described herein and illustrated in FIG. 4A, provide such improved properties. In some embodiments, method 400 is implemented using a computer system such as a gliflozin Sodium-Glucose Cotransport 2 inhibitor pharmaceutical composition OTC dispensing device 250 and/or personal device 102. As illustrated in FIG. 2, the gliflozin Sodium-Glucose Cotransport 2 inhibitor pharmaceutical composition OTC dispensing device (e.g., device 250) includes one or more processors (e.g., processor 274) and a memory (e.g., memory 192 and/or 290). The memory stores non-transitory instructions that, when executed by the one or more processors, perform a method.

In some embodiments of method 400, the gliflozin Sodium-Glucose Cotransport 2 inhibitor pharmaceutical composition includes an active ingredient having the structure:

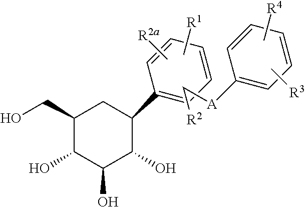

where,
R¹, R² and R^{2a} are independently hydrogen, OH, OR⁵, alkyl, CF₃, OCHF₂, OCF₃, SR⁵ⁱ or halogen, or two of R¹, R² and R^{2a} together with the carbons to which they are attached can form an annelated five, six or seven membered carbocycle or heterocycle which may contain 1 to 4 heteroatoms in the ring which are N, O, S, SO, and/or SO₂;
R³ and R⁴ are independently hydrogen, OH, OR^{5a}, OAryl, OCH₂Aryl, alkyl, cycloalkyl, CF₃, —OCHF₂, —OCF₃, halogen, —CN, —CO₂R^{5b}, —CO₂H, COR^{6b}, —CH(OH)R^{6c}, —CH (OR^{5h})R^{6d}, —CONR⁶R^{6a}, —NHCOR^{5c}, —NHSO₂R^{5d}, —NHSO₂Aryl, Aryl, —SR^{5e}, —SOR^{5f}, —SO₂R^{5g}, —SO₂Aryl, or a five, six or seven membered heterocycle which may contain 1 to 4 heteroatoms in the ring which are N, O, S, SO, and/or SO₂, or R³ and R⁴ together with the carbons to which they are attached form an annelated five, six or seven membered carbocycle or heterocycle which may contain 1 to 4 heteroatoms in the ring which are N, O, S, SO, and/or SO₂;
R⁵, R^{5a}, R^{5b}, R^{5c}, R^{5d}, R^{5e}, R^{5f}, R^{5g}, R^{5h} and R⁵ⁱ are independently alkyl;
R⁶, R^{6a}, R^{6b}, R^{6c}, and R^{6d} are independently hydrogen, alkyl, aryl, alkylaryl or cycloalkyl, or R⁶ and R^{6a} together with the nitrogen to which they are attached form an annelated five, six or seven membered heterocycle which may contain 1 to 4 heteroatoms in the ring which are N, O, S, SO, and/or SO₂;
A is O, S, NH, or (CH₂)ₙ, where n is 0-3, or a pharmaceutically acceptable salt, stereoisomer, or prodrug ester thereof;
with the proviso that where A is (CH₂)ₙ where n is 0, 1, 2, or 3 or A is O, and at least one of R¹, R², and R^{2a} is OH or OR⁵, then at least one of R¹, R², and R^{2a} is CF₃, OCF₃, or OCHF₂ and/or at least one of R³ and R⁴ is CF₃, —OCHF₂, —OCF₃, —CN, —CO₂Rb CH(OR^{5h})R^{6d}, CH(OH)R^{6c}, COR^{6b}, —NHCOR^{5c}, —NHSO₂R^{5d}, —NHSO₂Aryl, Aryl, —SR^{5e}, —SOR^{5f}, —SO₂R^{5g} or —SO₂Aryl.

In some embodiments of method 400, the gliflozin Sodium-Glucose Cotransport 2 inhibitor pharmaceutical composition includes dapagliflozin as an active agent. In some embodiments, the active agent is a pharmaceutically acceptable salt of dapagliflozin. In some embodiments, the gliflozin Sodium-Glucose Cotransport 2 inhibitor pharmaceutical composition includes dapagliflozin propanediol as an active agent.

In some embodiments, the gliflozin Sodium-Glucose Cotransport 2 inhibitor pharmaceutical composition includes dapagliflozin (e.g., (2S,3R,4R,5S,6R)-2-[4-chloro-3-[(4-ethoxyphenyl)methyl]phenyl]-6-(hydroxymethyl)oxane-3,4,5-triol), empagliflozin (e.g., (2S,3R,4R,5S,6R)-2-[4-chloro-3-[[4-[(3S)-oxolan-3-yl]oxyphenyl]methyl]phenyl]-6-(hydroxymethyl)oxane-3,4,5-triol), canagliflozin (e.g., (2S,3R,4R,5S,6R)-2-[3-[[5-(4-fluorophenyl)thiophen-2-yl]methyl]-4-methylphenyl]-6-(hydroxymethyl)oxane-3,4,5-triol), ertugliflozin (e.g., (1S,2S,3S,4R,5S)-5-[4-chloro-3-[(4-ethoxyphenyl)methyl]phenyl]-1-(hydroxymethyl)-6,8-dioxabicyclo[3.2.1]octane-2,3,4-triol), sotagliflozin (e.g., (2S,3R,4R,5S,6R)-2-[4-chloro-3-[(4-ethoxyphenyl)methyl]phenyl]-6-methylsulfanyloxane-3,4,5-triol), and/or ipragliflozin (e.g., (2S,3R,4R,5S,6R)-2-[3-(1-benzothiophen-2-ylmethyl)-4-fluorophenyl]-6-(hydroxymethyl)oxane-3,4,5-triol) as an active agent. These, and other, gliflozin Sodium-Glucose Cotransport 2 inhibitors are described, for example, in Dekkers et al., Curr. Diabetes Reports, 18:27 (2018), the content of which is hereby incorporated by reference.

In some embodiments, the gliflozin Sodium-Glucose Cotransport 2 inhibitor pharmaceutical composition includes any compound disclosed in U.S. Pat. No. 7,851,502, entitled “Pharmaceutical formulations containing an SGLT2 inhibitor,” which is hereby incorporated by reference. In some embodiments, the gliflozin Sodium-Glucose Cotransport 2 inhibitor pharmaceutical composition includes any compound disclosed in U.S. Pat. No. 9,834,533, entitled “Process for preparing SGLT2 inhibitors and intermediates thereof,” which is hereby incorporated by reference. In some embodiments, the gliflozin Sodium-Glucose Cotransport 2 inhibitor pharmaceutical composition includes any compound disclosed in U.S. Pat. No. 9,845,303, entitled “Process for the preparation of dapagliflozin,” which is hereby incorporated by reference.

In some embodiments, the gliflozin Sodium-Glucose Cotransport 2 inhibitor pharmaceutical composition includes any compound disclosed in U.S. Pat. No. 8,853,412, entitled “Pyrrolidinone derivatives as GPR119 modulators for the treatment of diabetes, obesity, dyslipidemia and related disorders,” which is hereby incorporated by reference.

In some embodiments, the gliflozin Sodium-Glucose Cotransport 2 inhibitor pharmaceutical composition includes any compound disclosed in U.S. Pat. No. 8,999,941, entitled “Crystalline and non-crystalline forms of SGLT2 inhibitors,” which is hereby incorporated by reference. In some embodiments, the gliflozin Sodium-Glucose Cotransport 2 inhibitor pharmaceutical composition includes any compound disclosed in U.S. Pat. No. 9,695,159, entitled “Process for preparation of canagliflozin,” which is hereby incorporated by reference

In some embodiments, the gliflozin Sodium-Glucose Cotransport 2 inhibitor pharmaceutical composition includes any compound disclosed in U.S. Pat. No. 9,192,617, entitled “Pharmaceutical composition, methods for treating, and the uses thereof,” which is hereby incorporated by reference. In some embodiments, the gliflozin Sodium-Glucose Cotransport 2 inhibitor pharmaceutical composition includes any compound disclosed in U.S. Pat. No. 9,902,751, entitled “Process for the preparation of empagliflozin,” which is hereby incorporated by reference.

In some embodiments, the gliflozin Sodium-Glucose Cotransport 2 inhibitor pharmaceutical composition includes any compound disclosed in U.S. Pat. No. 6,515,177, WO/2003/099836, U.S. PG Pub. No. 2006/0194809, U.S. PG Pub. No. 2006/0063722 A1, WO/2002/083066, U.S. PG Pub. No. 2003/0064935, U.S. Pat. No. 6,774,112, U.S. PG Pub. No. 2005/0209166, U.S. PG Pub. No. 2006/0074031, U.S. PG Pub. No. 2006/0035841, U.S. PG Pub. No. 2006/0009400, U.S. PG Pub. No. 2006/0025349, U.S. PG Pub. No. 2006/0122126, U.S. PG Pub. No. 2006/0019948, U.S. PG Pub. No. 2006/0194809, U.S. Pat. Nos. 6,908,905, 6,815,428, 6,555,519, 6,683,056, EP 598359 A1, JP 035988, U.S. Pat. No. 5,731,292, EP 0850948 A1, U.S. Pat. No. 6,048,842, JP 09188625 A, JP 09124685 A, JP 09124684, EP 773226 A1, U.S. Pat. No. 5,767,094, JP 08027006 A, EP 684254 A1, JP 10245391 (Dainippon), U.S. PG Pub. No. 2005/0233982 (Boehringer Ingelheim Corp.), U.S. PG Pub. No. 2005/0119192 (Kissei Pharmaceutical Co.), WO/2006/035796 (Kissei Pharmaceutical Co.), JP 2006/117651 (Taisho Pharmaceutical Co.), JP 2004/4359630 (Yamanouchi Pharmaceutical Co.), WO/2006/080421 (Chugai Seiyaku Kabushiki Kaishi), U.S. PG Pub. No. 2005/0233988 (Tanabe Seiyaku Co.), WO/2005/012321 (Tanabe Seiyaku Co.), U.S. Pat. No. 7,015,201 (Ajinomoto Co.), WO 2006/058597 (Merck Patent GmbH), WO 2006/011469 (Chugai Seiyaku Kabushiki Kaisha), U.S. PG Pub. No. 2003/0195235 (Johnson & Johnson), and WO 2006/037537 (Boehringer Ingelheim).

In some embodiments, the SGLT2 inhibitor is chosen from those disclosed in Tsujihara, K. et al., Chem. Pharm. Bull., 44:1174-1180 (1996); Hongu, M. et al., Chem. Pharm. Bull., 46:22-33 (1998); Hongu, M. et al., Chem. Pharm. Bull., 46:1545-1555 (1998); and Oku, A. et al., Diabetes, 48:1794-1800 (1999).

In some embodiments, the SGLT2 inhibitor may be dapagliflozin (FARXIGA®), canagliflozin (INVOKANA®), empagliflozin (JARDIANCE®), ertugliflozin (STEGLATRO®), sotagliflozin, ipragliflozin, tofogliflozin, or luseogliflozin, or a pharmaceutically acceptable salt, solvate, mixed solvate, complex, or prodrug of any of the foregoing.

In some embodiments, the SGLT2 inhibitor is dapagliflozin, or a pharmaceutically acceptable salt, solvate, mixed solvate, complex, or prodrug thereof, such as described in U.S. Pat. Nos. 6,414,126 and 6,515,117, which are incorporated by reference in their entireties.

In some embodiments, e.g., of method 400, a subject that has not received an over-the-counter provision of the gliflozin Sodium-Glucose Cotransport 2 inhibitor pharmaceutical composition will register as a new user, and the device will create a corresponding user profile (e.g., regardless of whether the subject previously received a prescription provision of the gliflozin Sodium-Glucose Cotransport 2 inhibitor pharmaceutical composition). In some embodiments, the subject will register as a returning customer, e.g., if the subject has previously received an over-the-counter provision of the gliflozin Sodium-Glucose Cotransport 2 inhibitor pharmaceutical composition and a corresponding user profile 234 already exists for the user. In some embodiments, when the subject is a returning customer and has previously qualified for an over-the-counter provision of the gliflozin Sodium-Glucose Cotransport 2 inhibitor pharmaceutical composition, the device will initiate a requalification process as further described below.

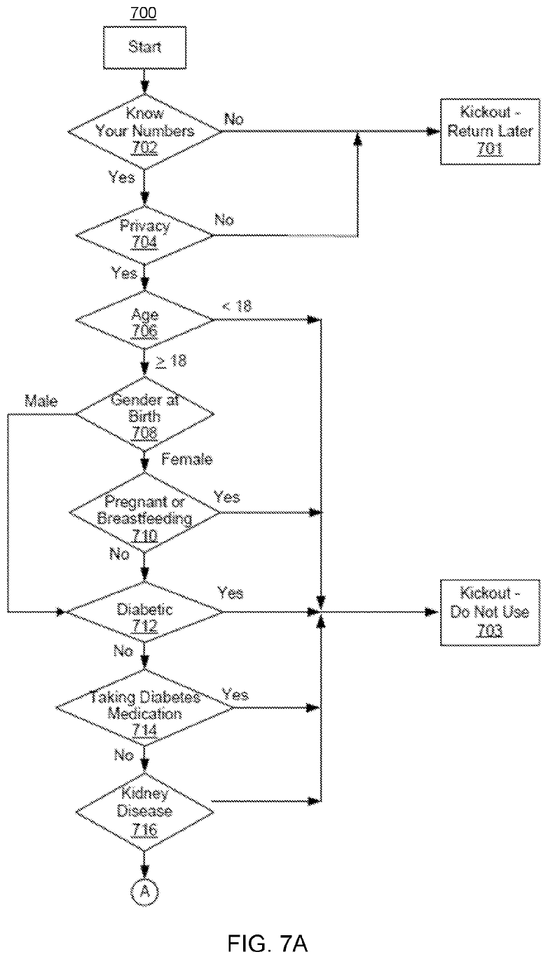

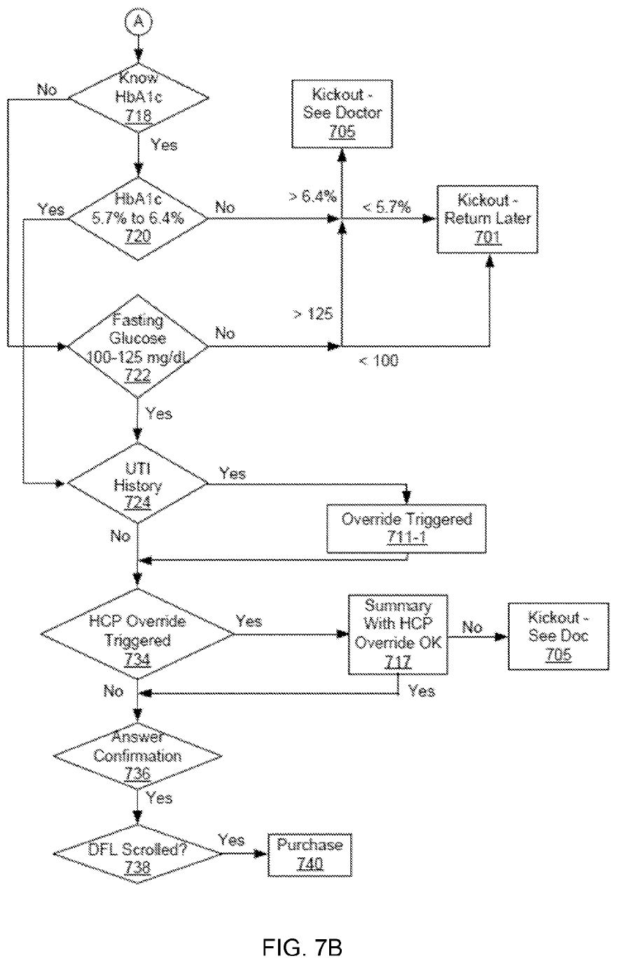

In some embodiments, the subject is prompted (e.g., as illustrated at step 704 of the example embodiment illustrated in FIG. 7) to confirm that they have adequate privacy to provide sensitive medical information, prior to proceeding with the process. Similarly, in some embodiments, the subject is prompted to confirm that they are in possession of medical information required to complete the qualification process (e.g., as illustrated at step 702 of the example embodiment illustrated in FIG. 7).

### Method 400—Initial Qualification

Referring to block 402 of FIG. 4A, method 400 includes obtaining an assessment information set about the subject, e.g., via a first computer system having a processor programed to obtain the assessment information set. In some embodiments, the obtaining includes conducting a first survey of the subject. By way of the first survey, a first plurality of survey results to survey questions 208, 212 (e.g., one or more of the survey questions set forth in Table 1) are obtained (e.g., the device transmits one or more survey questions to the user, prompting a response, and then receives a response to the one or more survey questions back from the subject). In some embodiments, the first survey results include some or all of the characteristics listed in Table 1. For example, in some embodiments, the first plurality of survey results includes 2, 3, 4, 5, 6, 7, 8, 9, 10, 11, or all 12 of the characteristics listed in Table 1. In one embodiment, the first survey questions 208, 212 and results include at least characteristics 1-8 as provided in Table 1. In one embodiment, the first survey questions 208 and 212 and results include at least characteristics 1, 2, and 4-8 as provided in Table 1.

It will be appreciated that the survey questions 208, 212 and filters 216, 222 applied to the survey answers thereof may vary depending upon the gliflozin Sodium-Glucose Cotransport 2 inhibitor pharmaceutical composition being distributed. This is due to differences in the contra-indication profiles of the various the gliflozin Sodium-Glucose Cotransport 2 inhibitor pharmaceutical composition, e.g., due to different drug-drug interactions, routes of drug clearance, etc. of the different the gliflozin Sodium-Glucose Cotransport 2 inhibitor pharmaceutical composition.

In some embodiments, as exemplified in FIGS. 3B-3C and 3E and FIG. 7, the first survey results include an age of the subject, a gender of the subject at birth, whether the subject is pregnant or breastfeeding (when the subject is female), whether the subject has type I or type II diabetes, whether the subject is taking a diabetes medication, an indication of whether the subject has kidney disease, an indication of the subject's blood glucose level, (e.g., HbA1c, eAG, or fasting glucose level), and an indication of whether the subject has a history of urinary tract infections.

In some embodiments, as exemplified in FIG. 3D, the first survey results include an age of the subject, a gender of the subject at birth, whether the subject has type I or type II diabetes, whether the subject is taking a diabetes medication, an indication of whether the subject has kidney disease, an indication of the subject's blood glucose level, (e.g., HbA1c, eAG, or fasting glucose level), and an indication of whether the subject has a history of urinary tract infections.

In some embodiments, as exemplified in FIG. 3F, the first survey results include an age of the subject, a gender of the subject at birth, whether the subject is pregnant or breastfeeding (when the subject is female), whether the subject has type I or type II diabetes, whether the subject is taking a diabetes medication, an indication of whether the subject has kidney disease, an indication of the subject's blood glucose level, (e.g., HbA1c, eAG, or fasting glucose level), an indication of whether the subject has a history of urinary tract infections, an indication of whether the subject has an increased risk for bone fractures, whether the subject is taking a medication that interacts with canagliflozin, and an indication of whether the subject has an increased risk of lower limb amputation.

In some embodiments, as exemplified in FIG. 3G, the first survey results include an age of the subject, a gender of the subject at birth, whether the subject is pregnant or breastfeeding (when the subject is female), whether the subject has type I or type II diabetes, whether the subject is taking a diabetes medication, an indication of whether the subject has kidney disease, an indication of the subject's blood glucose level, (e.g., HbA1c, eAG, or fasting glucose level), an indication of whether the subject has a history of urinary tract infections, and an indication of whether the subject has an increased risk of lower limb amputation.

Blocks 404-424. Referring to block 404 of FIG. 4A, an algorithm is applied to the assessment information set, e.g., via a second computer system (which is the same or different from the first computer system) having a processor programed to perform the algorithm.

Referring to Block 406 of FIG. 4A, the algorithm runs all or a portion of the assessment information set against a first set of assessment filters, wherein the subject is deemed not qualified for the over-the-counter provision of the gliflozin Sodium-Glucose Cotransport 2 inhibitor pharmaceutical composition when a filter in the first set of assessment filters is fired and the method is terminated without authorizing the over-the-counter provision of the gliflozin Sodium-Glucose Cotransport 2 inhibitor pharmaceutical composition to the subject. As previously described, the first set of filters comprises a subset of filters 216 of the first filter category class 214. When a respective filter in the first set of filters is fired (e.g., when a survey result indicates that a triggering condition 218 has been met), the subject is deemed not qualified for delivery of the gliflozin Sodium-Glucose Cotransport 2 inhibitor pharmaceutical composition and the method is terminated without authorization of the gliflozin Sodium-Glucose Cotransport 2 inhibitor pharmaceutical composition. Examples of filter combinations for the first set of filters are illustrated in FIGS. 3B-3G. Further details on implementing these filters, e.g., including example triggering conditions, are described below, e.g., in the “Contraindication” section.

In some embodiments, the first set of filters of the first category class 214 includes some or all of the filters 216 listed in Table 2. For example, in some embodiments, the first set of filters results includes 2, 3, 4, 5, or all 6 of the filters listed in Table 2. In some embodiments, the first set of filters results includes at least filters 1a and 3a-6a as listed in Table 2. It is contemplated that the first set of filters includes any sub-set of filters 216 provided in Table 2. Likewise, the skilled artisan may know of other filters 216, not provided in Table 2, which may be combined with any subset of the filters 216 provided in Table 2 to form the first set of filters results used in the methods described herein. For brevity, all possible combinations of the filters 216 provided in Table 2 are not specifically delineated here.

Referring to block 408 of FIG. 4A, the method also includes running all or a portion of the first survey results against a second set of filters of a second category class 220. When a respective filter in the second set of filters is fired, the subject is provided with a warning 226 corresponding to the respective filter (e.g., filter warning 228-1 corresponds to filter 222-1). In some embodiments, the warning 226 is provided as a next step, e.g., prior to applying survey results to any subsequent filters, after the corresponding filter is fired. For example, with respect to FIG. 7B, in some embodiments, e.g., when the UTI history filter is triggered at 724, the device would provide the subject with a warning prior to proceeding to a subsequent filter or later portion of the algorithm, e.g., requiring the subject confirm they have discussed their history of UTIs with a health care provider and the healthcare provider still recommends taking a gliflozin Sodium-Glucose Cotransport 2 inhibitor pharmaceutical composition. In some embodiments the warning 226 is provided after applying survey results to all subsequent filters. For example, as illustrated in FIG. 7B, in some embodiments, e.g., when the UTI history filter is triggered at 724, the device would proceed to a subsequent filter (if present) prior to transmitting a warning to the subject, and then transmit all warnings corresponding to filters of the second category class, at step 734, after survey results have been applied to all subsequent filters.

In some embodiments, the second set of filters of the second category class 216 includes some or all of the filters 220 listed in Table 3. For example, in some embodiments, the first set of filters results includes one or both filters listed in Table 3. In some embodiments, the second set of filters results includes at least filter 1b as listed in Table 3. It is contemplated that the second set of filters includes any sub-set of filters 222 provided in Table 3. Likewise, the skilled artisan may know of other filters 222, not provided in Table 3, which may be combined with any subset of the filters 222 provided in Table 3 to form the second set of filters results used in the methods described herein. For brevity, all possible combinations of the filters 222 provided in Table 3 are not specifically delineated here. Examples of filter combinations for the second set of filters are illustrated in FIGS. 3B-3G. Further details on implementing these filters, e.g., including example triggering conditions, are described below, e.g., in the “Risk Factor” section.

Referring to block 410 of FIG. 4A, the algorithm includes obtaining when a respective filter in the second set of assessment filters is fired, acknowledgment from the subject confirming that the subject has acknowledged the risk factor associated with each warning 226 issued to the subject by any filter 222 in the second set of assessment filters 216.

Referring to blocks 412-424 of FIG. 4A, the algorithm proceeds to a fulfillment process when no filter 216 in the first plurality of filters has been fired and the subject has acknowledged each warning 226 associated with each filter 222 in the second plurality of filters that was fired. In some embodiments, the fulfillment process includes storing (414) an indication, e.g., in a user profile 234, of an initial authorization date and/or destination (416) for the gliflozin Sodium-Glucose Cotransport 2 inhibitor pharmaceutical composition. The initial order date is utilized, for example, to verify at least a refill status of a provision of the gliflozin Sodium-Glucose Cotransport 2 inhibitor pharmaceutical composition. The initial order date is also utilized, for example, to verify at least an elapsed period of time between an initial order and a future re-order. Such verification is required in order to ensure that certain tests (e.g., blood glucose tests) are taken regularly.

In some embodiments, the fulfillment process includes communicating (420) an over-the-counter drug facts label 230 for the gliflozin Sodium-Glucose Cotransport 2 inhibitor pharmaceutical composition to the subject. In some embodiments, the drug facts label is communicated to the subject in real-time, e.g., within the same user interface as used for the qualification process. In some embodiments, the over-the-counter drug facts label 230 specifies what the gliflozin Sodium-Glucose Cotransport 2 inhibitor pharmaceutical composition is for (e.g., to lower blood sugar, to treat prediabetes, to delay onset of diabetes, etc.) and any risks associated with taking the gliflozin Sodium-Glucose Cotransport 2 inhibitor pharmaceutical composition (e.g., drug-drug interactions, pharmacokinetic interactions, adverse reactions, etc.).

In some embodiments, the fulfillment process includes authorizing (418) provision of the gliflozin Sodium-Glucose Cotransport 2 inhibitor pharmaceutical composition to the subject. In some embodiments, the authorization occurs upon confirmation (422) from the subject that the over-the-counter drug facts label 230 has been received and read by the subject. In some embodiments, the authorization includes (416) a destination associated with the subject. In some embodiments, the destination associated with the subject is stored in the user profile 234. In some embodiments, the destination associated with the subject is a physical address including a street address, a Post Office box, a pharmacy associated with the subject, a health care provider associated with the subject, and/or one or more coordinates (e.g., longitude, latitude, elevation). In some embodiments, the fulfillment process includes coordinating shipping the gliflozin Sodium-Glucose Cotransport 2 inhibitor pharmaceutical composition to a physical address associated with the subject (424). In some embodiments, the provision of the gliflozin Sodium-Glucose Cotransport 2 inhibitor pharmaceutical composition is shipped to a pharmacy associated and/or a location associated with a health care provider of the subject and/or an office of a medical practitioner associated with the subject. Further details on the provision of the gliflozin Sodium-Glucose Cotransport 2 inhibitor pharmaceutical composition, e.g., as to the identity and dosage of the active ingredient, is provided below, e.g., in the “Dosage” section.

### Assessment Information Set

As described above, in some embodiments, the methods described herein include obtaining an assessment information set about the subject. In some embodiments, the obtaining includes providing an assessment survey to the subject. In some such embodiments, the assessment information set corresponding to the assessment survey questions 208, 212 (e.g., one or more of the assessment survey questions set forth in Table 1) is obtained (e.g., the device transmits one or more assessment survey questions to the user, prompting a response, and then receives a response to the one or more assessment survey questions back from the subject). In some embodiments, the assessment information set includes some of or all the characteristics listed in Table 1. For example, in some embodiments, the assessment information set includes 2, 3, 4, 5, 6, 7, 8, 9, 10, 11, or all 12 characteristics listed in Table 1. In one embodiment, the assessment survey questions 208, 212 and assessment information set include at least characteristics 1-7 as provided in Table 1. In one embodiment, the assessment survey questions 208, 212 and assessment information set include at least characteristics 1, 2, and 4-7 as provided in Table 1.

It will be appreciated that the assessment survey questions 208, 212 and assessment filters 216, 222 applied to the survey answers (e.g., to the assessment information set) thereof obtained may vary depending upon the gliflozin Sodium-Glucose Cotransport 2 inhibitor pharmaceutical composition being distributed. This is due to differences in the contra-indication profiles of the various the gliflozin Sodium-Glucose Cotransport 2 inhibitor pharmaceutical composition, e.g., due to different drug-drug interactions, routes of drug clearance, etc. of the different gliflozin Sodium-Glucose Cotransport 2 inhibitor pharmaceutical composition.

In some embodiments, the assessment survey includes questions that elicit responses providing some of or all the characteristics listed in Table 1. In some embodiments, the survey includes questions corresponding to each of the survey results (e.g., as part of the assessment information set) required for the methods described herein. In other embodiments, the survey includes questions corresponding to only a subset of the survey results required for the methods described herein. In such embodiments, other survey results required for the methods described herein are acquired through other means (e.g., upon registration/subscription for a service associated with qualifying the subject for over-the-counter medication, from a healthcare provider, from a prior survey, from a database associated with a pharmacy, from an electronic health record associated with the subject, from the subject profile data store 232, etc.) For example, in some embodiments, the subject provides a personal medical identification associated with an insurer, a hospital, or other healthcare provider and information about the subject required for the methods described herein, e.g., one or more survey results, is acquired from a preexisting database associated with the personal medical identification (e.g., a last blood sugar measurement determined for the subject). The same applies to the reassessment survey 210 and corresponding results applied to the assessment survey 206.

TABLE 1
Example Assessment Survey Question Characteristics

| Result | Example Characteristics |
|---|---|
| 1 | an age of the subject |
| 2 | a gender of the subject at birth |
| 3 | when the subject is female, whether the subject is |
| | pregnant or breastfeeding |
| 4 | whether the subject has type I or type II diabetes |
| 5 | whether the subject is taking a diabetes medication |
| 6 | an indication of whether the subject has kidney disease |
| 7 | an indication of the subject's blood glucose level, |
| | (e.g., HbA1c, eAG, or fasting glucose level) |
| 8 | an indication of whether the subject has a history |
| | of urinary tract infections |
| 9 | an indication of whether the subject is at increased |
| | risk of bone fractures |
| 10 | whether the subject is taking a medication that interacts |
| | with the gliflozin Sodium-Glucose Cotransport 2 inhibitor |
| | pharmaceutical composition |
| 11 | an indication of whether the subject is at increased |
| | risk for lower limb amputation |
| 12 | whether the subject is allergic to the gliflozin Sodium- |
| | Glucose Cotransport 2 inhibitor pharmaceutical composition |
| 13 | An indication of the subject's body mass index (BMI) |

It is contemplated that, in some embodiments, any one or more of the survey questions 208, 212 provided in Table 1 will not be included in the assessment survey (e.g., will not be used for the assessment). For example, in some embodiments, a characteristic associated with a particular survey question will be informative when qualifying a subject for one particular gliflozin Sodium-Glucose Cotransport 2 inhibitor pharmaceutical composition but not for another gliflozin Sodium-Glucose Cotransport 2 inhibitor pharmaceutical composition. For instance, a survey question is queried for canagliflozin qualifying surveys but not for empagliflozin qualifying surveys. The skilled artisan will recognize that different gliflozin Sodium-Glucose Cotransport 2 inhibitor pharmaceutical composition carry different risk and drug interaction profiles. Accordingly, survey information required for qualifying a subject for access to one gliflozin Sodium-Glucose Cotransport 2 inhibitor pharmaceutical composition with a known adverse drug interaction may not be necessary for qualifying the same subject for access to a second gliflozin Sodium-Glucose Cotransport 2 inhibitor pharmaceutical composition.

Accordingly, it is contemplated that the assessment survey questions 208 include any subset of survey results provided in Table 1. For brevity, all possible combinations of the survey questions 208, 212 provided in Table 1 are not specifically delineated here. However, the skilled artisan will easily be able to envision any particular subset of the survey questions 208, 212 provided in Table 1. Likewise, the skilled artisan may know of other survey questions (e.g., not provided in Table 1) that may be combined with any subset of the survey questions provided in Table 1 to form the assessment survey questions used in the methods described herein.

In some embodiments, the assessment and/or reassessment survey is conducted by transmitting a plurality of questions to the subject, e.g., some or all of the survey questions, and receiving answers to the plurality of survey questions before applying any of the answers to respective filters. For example, with reference to the workflow in FIG. 7, the device transmits questions relating to all of the filters of the first category class, all of the filters of the second category class, or all of the filters in the workflow (e.g., as a virtual survey where all of the questions are displayed in a single user interface, or as a series of questions displayed in consecutive user interfaces). After receiving answers to all of the assessment and/or reassessment survey questions, the device then applies the answers to all of the filters (e.g., sequentially or concurrently) to determine whether the subject is qualified to receive provision of the gliflozin Sodium-Glucose Cotransport 2 inhibitor pharmaceutical composition. In alternative embodiments, the device transmits questions relating to just those filters of the first category class for which it could not obtain answers to the questions from an electronic database associated with the subject, such as electronic health record of the subject, and just those filters of the second category class it could not obtain answers to the questions from an electronic database associated with the subject (e.g., as a virtual survey where such unanswered questions are displayed in a single user interface, or as a series of questions displayed in consecutive user interfaces). After receiving answers to all of the assessment and/or reassessment survey questions, the device then applies the answers to all of the filters (e.g., sequentially or concurrently) to determine whether the subject is qualified to receive provision of the gliflozin Sodium-Glucose Cotransport 2 inhibitor pharmaceutical composition.

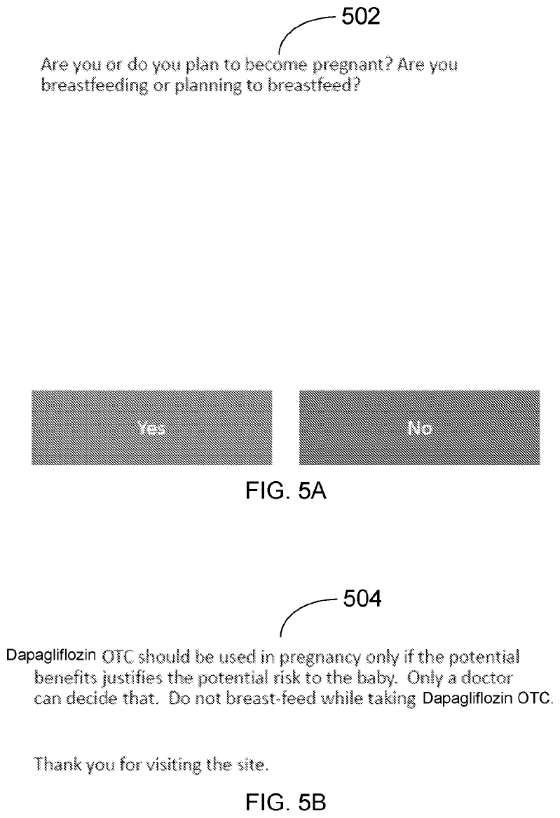

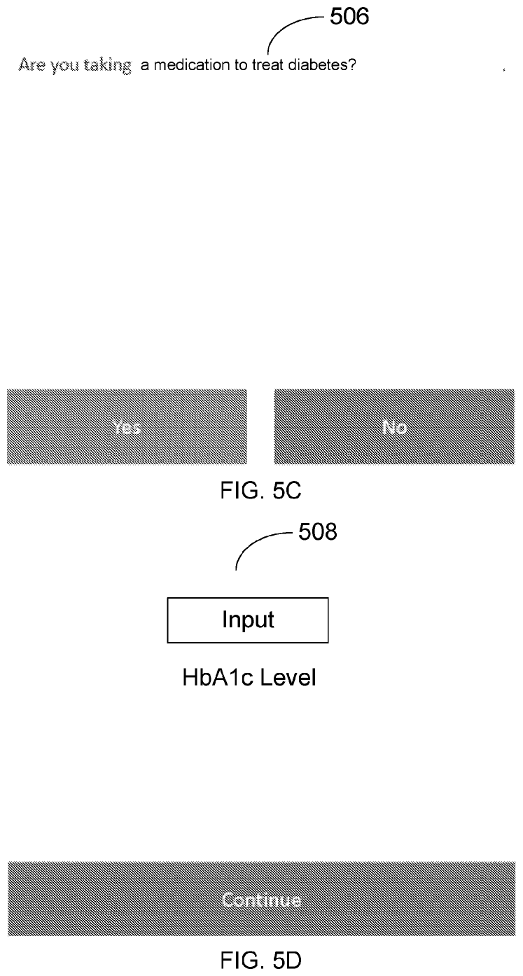

In some embodiments, the assessment and/or reassessment survey is conducted in a serial fashion, e.g., by transmitting a first question or a first group of survey questions (e.g., associated with a single filter) to the subject, receiving an answer to the single survey question or small group of survey questions, and applying the answer or answers to a filter, prior to transmitting a second question or second group of questions to the subject. For example, with reference to the workflow in FIG. 7, in some embodiments the device transmits a first question to the subject, relating to the age of the subject. After receiving the answer to the survey question, the device applies the answer to an age filter (706). If the age filter is fired (e.g., in response to an answer of “15 years old”), the device terminates (701-1) the process, and optionally provides the user with a message relating to why they are being denied a provision of the gliflozin Sodium-Glucose Cotransport 2 inhibitor pharmaceutical composition (e.g., as illustrated in FIG. 5B, message 504, advising the subject that taking the gliflozin Sodium-Glucose Cotransport 2 inhibitor pharmaceutical composition creates a risk for the fetus/baby of a pregnant woman), a suggestion for following-up with a medical professional (e.g., as illustrated in FIG. 7B, when the survey answers indicate that the subject is already diabetic, the device terminates the process (705) and advises that the subject consult with a doctor), and/or a suggestion to make a lifestyle change to treat or manage their blood sugar.

### Assessment Filters

Referring to blocks 406-410 of FIG. 4A, an algorithm is applied to assessment information set. The algorithm first runs (406) all or a portion of the assessment information set against a first set of assessment filters 214 (e.g., filters 216 of a first category class). As previously described, the first set of assessment filters includes a subset of filters 216 of the first filter category class 214. When a respective filter in the first set of assessment filters is fired (e.g., when a survey result indicates that a triggering condition 218 has been met), the subject is deemed not qualified for a provision of the gliflozin Sodium-Glucose Cotransport 2 inhibitor pharmaceutical composition and the method is terminated without authorization of the gliflozin Sodium-Glucose Cotransport 2 inhibitor pharmaceutical composition to the subject. Examples of specific filters 216 in the first set of assessment filters and their exemplary triggering conditions 218 that cause the corresponding filter to fire are detailed below.

In some embodiments, the first set of assessment filters 214 includes some of or all the filters 216 listed in Table 2. For example, in some embodiments, the first set of assessment filters includes 2, 3, 4, 5, 6, 7, or all 8 of the filters listed in Table 2.

TABLE 2
Examples of Assessment Filters of the First Category Class

| Filter | Example Criteria |
|---|---|
| 1a | an age filter 216-1 |
| 2a | a pregnancy filter 216-2 |
| 3a | a diabetes filter 216-3 |
| 4a | a diabetes medication filter 216-4 |
| 5a | a kidney disease filter 216-5 |
| 6a | a blood glucose filter 216-6 |
| 7a | an allergy filter 216-7 |
| 8a | a body mass index filter 216-8 |
| 9a | A diabetes risk factor filter |

In one embodiment, the first set of assessment filters includes at least filters 1a and 3a-6a as provided in Table 2. In another embodiment, the first set of assessment filters includes at least filters 1a-6a as provided in Table 2. In one embodiment, the first set of assessment filters includes at least filters 1a, 3a-6a, and 9a as provided in Table 2. In one embodiment, the first set of assessment filters includes at least filters 1a-6a and 9a as provided in Table 2. It is contemplated that, in some embodiments, any one or more of the filters 216 provided in Table 2 will not be included in the first set of assessment filters. For example, in some embodiments, a characteristic associated with a particular survey result will be informative when qualifying a subject for one particular gliflozin Sodium-Glucose Cotransport 2 inhibitor pharmaceutical composition but not for another gliflozin Sodium-Glucose Cotransport 2 inhibitor pharmaceutical composition.

Accordingly, it is contemplated that the first set of assessment filters includes any sub-set of filters 216 provided in Table 2. Likewise, the skilled artisan may know of other filters 216, not provided in Table 2, which may be combined with any subset of the filters 216 provided in Table 2 to form the first set of assessment filters results used in the methods described herein. For brevity, all possible combinations of the filters 216 provided in Table 2 are not specifically delineated here.

In some embodiments, the first set of assessment filters includes an age filter (e.g., age filter 216-1 in FIG. 3 and/or filter 1a in Table 2). The age filter is configured to be fired at least when the assessment information set indicates the age of the subject fails to satisfy an age threshold for receiving the gliflozin Sodium-Glucose Cotransport 2 inhibitor pharmaceutical composition. In some embodiments, the age filter is fired when the assessment information set indicates the subject is less than 18 years of age. In other embodiments, the age filter is fired when the assessment information set indicates the subject is less than, e.g., 15, 16, 17, 19, 20, 21, 25, 30, 35, 40, 45, or 50 years of age. If the age filter is fired, the subject is not permitted to obtain the gliflozin Sodium-Glucose Cotransport 2 inhibitor pharmaceutical composition over-the-counter (e.g., the method is terminated without authorizing provision of the gliflozin Sodium-Glucose Cotransport 2 inhibitor pharmaceutical composition to the subject). In some embodiments, an age filter is applied as a second filter category class 220 type filter, e.g., when a particular range of subject age is a risk factor, rather than a contraindication, for use of the gliflozin Sodium-Glucose Cotransport 2 inhibitor pharmaceutical composition.

In some embodiments, the first set of assessment filters includes, when the subject is a female, a pregnancy filter (e.g., pregnancy filter 216-2 in FIG. 3 and/or filter 2a in Table 2). The pregnancy filter is configured to be fired at least when the assessment information set indicates the subject is pregnant or breastfeeding. In some embodiments, the pregnancy filter is also configured to be fired when the assessment information set indicates the subject is female and planning on becoming pregnant. When the pregnancy filter is fired, the subject is not permitted to obtain the gliflozin Sodium-Glucose Cotransport 2 inhibitor pharmaceutical composition over-the-counter (e.g., the method is terminated without authorizing provision of the gliflozin Sodium-Glucose Cotransport 2 inhibitor pharmaceutical composition). For example, the device transmits prompt 502, as illustrated in FIG. 5A, to the subject and the device applies the subject's answer to the pregnancy filter. If the subject's answer indicates that they are pregnant, they are planning on being pregnant, they are breastfeeding, or they are planning to breastfeeding, the pregnancy filter is fired, and the method is terminated without authorizing provision of the gliflozin Sodium-Glucose Cotransport 2 inhibitor pharmaceutical composition to the subject. In some embodiments, the device transmits a message explaining why authorization was denied, e.g., message 504 illustrated in FIG. 5B. In some embodiments, a pregnancy filter is applied as a second filter category class 220 type filter, e.g., when pregnancy, breastfeeding, and/or planning to become pregnant are risk factors, rather than contraindications, for use of the gliflozin Sodium-Glucose Cotransport 2 inhibitor pharmaceutical composition.

In some embodiments the first set of assessment filters includes a diabetes filter (e.g., diabetes filter 216-3 in FIG. 3 and/or filter 3a in Table 2). The diabetes filter is configured to be fired at least when the first plurality of survey results indicates that the subject has Type 1 or Type 2 diabetes. If the diabetes filter is fired, the subject is not permitted to obtain the gliflozin Sodium-Glucose Cotransport 2 inhibitor pharmaceutical composition over-the-counter (e.g., the method is terminated without authorizing provision of the gliflozin Sodium-Glucose Cotransport 2 inhibitor pharmaceutical composition to the subject). In some embodiments, the diabetes filter is configured to be filed when the assessment information set indicates the subject was previously diagnosed with Type 1 or Type 2 diabetes. In some embodiments, the diabetes filter is also configured to be filed when the assessment information set indicates the subject has a blood sugar level falling within a diabetic range, e.g., a glycated hemoglobin (HbA1c) level of greater than 6.4% or a fasting glucose level of greater than 125 mg/dL. In some embodiments, the diabetes filter is configured to be fired when the assessment information set indicates the subject has an HbA1c level greater than a threshold value falling between 6.2% HbA1c and 6.6% HbA1c. For example, in some embodiments, the diabetes filter is configured to be fires when the assessment information set indicates the subject has an HbA1c level greater than 6.2% HbA1c, 6.3% HbA1c, 6.4% HbA1c, 6.5% HbA1c, or 6.6% HbA1c. In other embodiments, the diabetes filter is configured to be fired when the assessment information set indicates the subject has a fasting glucose level greater than a threshold value falling between 119 mg/dL and 131 mg/dL. For example, in some embodiments, the diabetes filter is configured to be fires when the assessment information set indicates the subject has a fasting glucose level greater than 119 mg/dL, 120 mg/dL, 121 mg/dL, 122 mg/dL, 123 mg/dL, 124 mg/dL, 125 mg/dL, 126 mg/dL, 127 mg/dL, 128 mg/dL, 129 mg/dL, 130 mg/dL, or 131 mg/dL.

In some embodiments, a diabetes filter (e.g., a Type I diabetes filter, a Type II diabetes filter, or a combined Type 1 and 2 diabetes filter) is applied as a second filter category class 220 type filter, e.g., when Type I diabetes and/or Type II diabetes is a risk factor, rather than a contraindication, for use of the gliflozin Sodium-Glucose Cotransport 2 inhibitor pharmaceutical composition over-the-counter. In some embodiments, separate Type 1 diabetes and Type 2 diabetes filters are implemented. In some embodiments, the separate Type 1 and Type 2 diabetes filters are of the same filter class (e.g., they are both of a first filter category class 214 or both of a second filter category class 220). In some embodiments, the separate Type 1 and Type 2 diabetes filters are of different filter category classes (e.g., the Type 1 diabetes filter is of a first category class 214 and the Type II diabetes filter is of a second category class 220).

In some embodiments, the first set of assessment filters includes a diabetes medication filter (e.g., diabetes medication filter 216-4 in FIG. 3 and/or filter 4a in Table 2). The diabetes medication filter is configured to be fired at least when the assessment information set indicates the subject is taking one or more compositions that lowers blood sugar, regardless of whether the composition was prescribed specifically for treatment of diabetes or not. Non-limiting examples of diabetes medications that may trigger a diabetes medication filter include a gliflozin Sodium-Glucose Cotransport 2 inhibitor pharmaceutical composition, insulin or an analog thereof, an amylinomimetic drug, an alpha-glucosidase inhibitor, a biguanide (e.g., metformin), a dopamine agonist, a dipeptidyl peptidase-4 (DPP-4) inhibitor, a glucagon-like peptide-1 receptor agonist (GLP-1 receptor agonist), a meglitinide, a sulfonylurea, and a thiazolidinedione. If the diabetes medication filter is fired, the subject is not permitted to obtain the gliflozin Sodium-Glucose Cotransport 2 inhibitor pharmaceutical composition over-the-counter (e.g., the method is terminated without authorizing provision of the gliflozin Sodium-Glucose Cotransport 2 inhibitor pharmaceutical composition to the subject). In some embodiments, a diabetes medication filter is applied as a second filter category class 220 type filter, e.g., when co-administration of a diabetes medication is a risk factor, rather than a contraindication, for use of the gliflozin Sodium-Glucose Cotransport 2 inhibitor pharmaceutical composition over-the-counter.

In some embodiments, the first set of assessment filters includes a kidney disease filter (e.g., kidney disease filter 216-5 in FIG. 3 and/or filter 5a in Table 2). In some embodiments, the kidney disease filter is fired when the first plurality of survey results indicates that the subject has kidney disease. In some embodiments, the kidney disease filter is configured to be fired when the assessment information set includes an indication that the subject has been diagnosed with kidney disease. In some embodiments, the kidney disease filter is configured to be fired when the assessment information set includes an indication that the subject is currently receiving kidney dialysis. Accordingly, in some embodiments, an assessment survey administered to the subject asks one or both of (i) whether the subject has been diagnosed with kidney disease, and (ii) whether the subject is receiving kidney dialysis. In some embodiments, separate filters are implemented for a kidney disease diagnosis and receipt of kidney dialysis, e.g., both of which are of the first category class. If the kidney disease filter is fired, the subject is not permitted to obtain the gliflozin Sodium-Glucose Cotransport 2 inhibitor pharmaceutical composition pharmaceutical composition over-the-counter (e.g., the method is terminated without authorizing provision of the gliflozin Sodium-Glucose Cotransport 2 inhibitor pharmaceutical composition to the subject). In some embodiments, one or more kidney disease filter is applied as a second filter category class 220 type filter, e.g., when kidney disease is a risk factor, rather than a contraindication, for use of the gliflozin Sodium-Glucose Cotransport 2 inhibitor pharmaceutical composition over-the-counter.

In some embodiments, the first set of assessment filters includes a blood glucose (or blood sugar) filter (e.g., blood glucose filter 216-6 in FIG. 3 and/or filter 6a in Table 2). In some embodiments, the blood glucose filter is configured to be fired when the first plurality of survey results indicates that the subject has a blood glucose level that is below a first baseline blood glucose level (e.g., corresponding to a blood glucose level that is indicative that the subject does not have prediabetes). In some embodiments, the blood glucose (or blood sugar) filter is also configured to be fired when the first plurality of survey results indicates that the subject has a blood glucose level that is above a ceiling blood glucose level (e.g., corresponding to a blood glucose level that is indicative that the subject has developed diabetes). If the blood glucose filter is fired, the subject is not permitted to obtain the gliflozin Sodium-Glucose Cotransport 2 inhibitor pharmaceutical composition pharmaceutical composition over-the-counter (e.g., the method is terminated without authorizing provision of the gliflozin Sodium-Glucose Cotransport 2 inhibitor pharmaceutical composition to the subject). In some embodiments, separate low blood glucose and high blood glucose filters are implemented. In some embodiments, one or more blood glucose filter is applied as a second filter category class 220 type filter, e.g., when a low and/or high blood glucose level is a risk factor, rather than a contraindication, for use of the gliflozin Sodium-Glucose Cotransport 2 inhibitor pharmaceutical composition over-the-counter.

However, in some embodiments where a separate diabetes filter (e.g., diabetes filter 216-3 in FIG. 3 and/or filter 3a in Table 2) is used, such a configuration where the blood glucose filter is configured to be fired when the first plurality of survey results indicates that the subject has a blood glucose level that is above a ceiling blood glucose level may be redundant with the diabetes filter. For example, depending upon the configuration of the diabetes filter it may have already been fired-thus already terminating the process—by the indication of blood sugar levels that would also cause the blood glucose filter to fire above such a ceiling threshold. Accordingly, in some embodiments, the diabetes filter and the blood glucose filter can be combined into a single filter that is configured to be fired when the first plurality of survey results indicates that the subject has a blood glucose level that is below a first threshold (e.g., indicating that the subject is not at least pre-diabetic) and when the first plurality of survey results indicates that the subject has a blood glucose level that is above a second threshold (e.g., indicating that the subject has diabetes). In some embodiments, such a combined filter is also configured to be fired when the first plurality of survey results indicates that the subject has been diagnosed as diabetic.

In some embodiments, the first baseline blood glucose level used in the blood glucose filter is 5.7% glycated hemoglobin (HbA1c), or an equivalent thereof (e.g., an equivalent eAG level). In other embodiments, the first baseline blood glucose level used in the blood glucose filter is from 5.3% HbA1c to 6.1% HbA1c, e.g., 5.3% HbA1c, 5.4% HbA1c, 5.5% HbA1c, 5.6% HbA1c, 5.7% HbA1c, 5.8% HbA1c, 5.9% HbA1c, 6.0% HbA1c, or 6.1% HbA1c. In other embodiments, the first baseline blood glucose level used in the blood glucose filter is from 5.4% HbA1c to 6.0% HbA1c. In other embodiments, the first baseline blood glucose level used in the blood glucose filter is from 5.5% HbA1c to 5.9% HbA1c. In other embodiments, the first baseline blood glucose level used in the blood glucose filter is from 5.6% HbA1c to 5.8% HbA1c.

In some embodiments, the first baseline blood glucose level used in the blood glucose filter is 100 mg/dL fasting glucose. In other embodiments, the first baseline blood glucose level used in the blood glucose filter is from 87.5 mg/dL to 112.5 mg/dL fasting glucose, e.g., 87.5 mg/dL, 90 mg/dL, 95 mg/dL, 100 mg/dL, 105 mg/dL, 110 mg/dL, or 112.5 mg/dL fasting glucose. In some embodiments, the first baseline blood glucose level used in the blood glucose filter is from 90 mg/dL to 110 mg/dL fasting glucose. In some embodiments, the first baseline blood glucose level used in the blood glucose filter is from 95 mg/dL to 105 mg/dL fasting glucose.

In some embodiments, the ceiling blood sugar level used in the first blood glucose filter is 6.4% glycated hemoglobin (HbA1c), or an equivalent thereof (e.g., an equivalent eAG level). In other embodiments, the ceiling blood sugar level used in the blood glucose filter is from 6.1% HbA1c to 6.7% HbA1c, e.g., 6.1% HbA1c, 6.2% HbA1c, 6.3% HbA1c, 6.4% HbA1c, 6.5% HbA1c, 6.6% HbA1c, or 6.7% HbA1c. In other embodiments, the ceiling blood sugar level used in the blood glucose filter is from 6.2% HbA1c to 6.6% HbA1c. In other embodiments, the first baseline blood glucose level used in the blood glucose filter is from 6.3% HbA1c to 6.5% HbA1c.

In some embodiments, the ceiling blood sugar level used in the first blood glucose filter is 125 mg/dL fasting glucose. In other embodiments, the ceiling blood sugar level used in the blood glucose filter is from 112.5 mg/dL to 137.5 mg/dL fasting glucose, e.g., 112.5 mg/dL, 115 mg/dL, 120 mg/dL, 125 mg/dL, 130 mg/dL, 135 mg/dL, or 137.5 mg/dL fasting glucose. In other embodiments, the ceiling blood sugar level used in the blood glucose filter is from 115 mg/dL to 135 mg/dL fasting glucose. In other embodiments, the ceiling blood glucose level used in the blood glucose filter is from 120 mg/dL to 130 mg/dL fasting glucose.

In some embodiments the first set of assessment filters includes an allergy filter (e.g., filter 7a in Table 2). The allergy filter is configured to be fired at least when the first plurality of survey results indicates that the subject is allergic to a component of the gliflozin Sodium-Glucose Cotransport 2 inhibitor pharmaceutical composition. In some embodiments, the adverse reaction filter is configured to be fired when the assessment information set indicates that the subject has developed an adverse reaction to the gliflozin Sodium-Glucose Cotransport 2 inhibitor pharmaceutical composition in the past. In some embodiments, the adverse reaction filter is configured to be fired when the assessment information set indicates that the subject has developed an adverse reaction to any gliflozin Sodium-Glucose Cotransport 2 inhibitor pharmaceutical composition in the past. If the allergy filter is fired, the subject is not permitted to obtain the gliflozin Sodium-Glucose Cotransport 2 inhibitor pharmaceutical composition over-the-counter (e.g., the method is terminated without authorizing provision of the gliflozin Sodium-Glucose Cotransport 2 inhibitor pharmaceutical composition to the subject). In some embodiments, an allergy filter is applied as a second filter category class 220 type filter, e.g., when the allergy is a risk factors, rather than contraindication, for use of the gliflozin Sodium-Glucose Cotransport 2 inhibitor pharmaceutical composition.

In some embodiments, the first set of assessment filters includes a body mass index (BMI) filter (e.g., filter 8a in Table 2). The BMI filter is configured to be fired at least when the assessment information set indicates the BMI of the subject is below a BMI threshold for receiving the gliflozin Sodium-Glucose Cotransport 2 inhibitor pharmaceutical composition. In some embodiments, the BMI filter is fired when the assessment information set indicates the subject's BMI is less than 25 kg/m². In other embodiments, the BMI filter is fired when the assessment information set indicates the subject's BMI is less than 20 kg/m², 21 kg/m², 22 kg/m², 23 kg/m², 24 kg/m², 26 kg/m², 27 kg/m², 28 kg/m², 29 kg/m², 30 kg/m², 31 kg/m², 32 kg/m², 33 kg/m², 34 kg/m², or 35 kg/m². In some embodiments, the BMI threshold is from 22.5 kg/m² to 27.5 kg/m². In other embodiments, the BMI threshold is from 20 kg/m² to 22.5 kg/m², from 20 kg/m² to 25 kg/m², from 20 kg/m² to 27.5 kg/m², from 20 kg/m² to 30 kg/m², from 22.5 kg/m² to 25 kg/m², from 22.5 kg/m² to 30 kg/m², from 22.5 kg/m² to 35 kg/m², from 25 kg/m² to 27.5 kg/m², from 25 kg/m² to 30 kg/m², or from 27.5 kg/m² to 30 kg/m². If the BMI filter is fired, the subject is not permitted to obtain the gliflozin Sodium-Glucose Cotransport 2 inhibitor pharmaceutical composition over-the-counter (e.g., the method is terminated without authorizing provision of the gliflozin Sodium-Glucose Cotransport 2 inhibitor pharmaceutical composition to the subject). In some embodiments, a BMI filter is applied as a second filter category class 220 type filter, e.g., when a particular range of subject BMI is a risk factor, rather than a contraindication, for use of the gliflozin Sodium-Glucose Cotransport 2 inhibitor pharmaceutical composition. In some embodiments, the assessment information set includes the height and weight of the subject, and the subject's BMI is determined therefrom. In some embodiments, the assessment information set includes the BMI of the subject. In some embodiments, the system displays a table correlating subject height and weight with BMI. In some embodiments, BMI is calculated differently based upon whether the subject is male or female.

In some embodiments, the first set of assessment filters includes a diabetes risk factor filter (e.g., filter 9a in Table 2). The diabetes risk factor filter is configured to be fired at least when the assessment information set indicates the subject does not have at least one supplemental risk factor for developing diabetes. In some embodiments, the supplemental risk factor is selected from an elevated body mass index (BMI), a first degree relative with type II diabetes, a history of hypertension, a history of dyslipidemia, prior gestational diabetes, and polycyctic ovary syndrome. In some embodiments, an elevated body mass index is a BMI of at least 25 kg/m². In some embodiments, an elevated body mass index is a BMI of at least 20 kg/m², 21 kg/m², 22 kg/m², 23 kg/m², 24 kg/m², 26 kg/m², 27 kg/m², 28 kg/m², 29 kg/m², 30 kg/m², 31 kg/m², 32 kg/m², 33 kg/m², 34 kg/m², or 35 kg/m². In some embodiments, an elevated body mass index is a BMI of at least a first threshold level of from 22.5 to 27.5. In other embodiments, the BMI threshold is from 20 kg/m² to 22.5 kg/m², from 20 kg/m² to 25 kg/m², from 20 kg/m² to 27.5 kg/m², from 20 kg/m² to 30 kg/m², from 22.5 kg/m² to 25 kg/m², from 22.5 kg/m² to 30 kg/m², from 22.5 kg/m² to 35 kg/m², from 25 kg/m² to 27.5 kg/m², from 25 kg/m² to 30 kg/m², or from 27.5 kg/m² to 30 kg/m². If the diabetes risk factor filter is fired, the subject is not permitted to obtain the gliflozin Sodium-Glucose Cotransport 2 inhibitor pharmaceutical composition over-the-counter (e.g., the method is terminated without authorizing provision of the gliflozin Sodium-Glucose Cotransport 2 inhibitor pharmaceutical composition to the subject). In some embodiments, a diabetes risk factor filter is applied as a second filter category class 220 type filter, e.g., when a particular range of subject BMI is a risk factor, rather than a contraindication, for use of the gliflozin Sodium-Glucose Cotransport 2 inhibitor pharmaceutical composition.

Contraindications described in the present disclosure are non-exhaustive. The skilled artisan may know of other contraindications for a particular gliflozin Sodium-Glucose Cotransport 2 inhibitor pharmaceutical composition and/or treat risk factors as contraindications dependent upon the intended use of the gliflozin Sodium-Glucose Cotransport 2 inhibitor pharmaceutical composition. In some embodiments, a contraindication for use of a prescription-strength pharmaceutical composition are treated only as risk factors, or not at all, when qualifying a subject for a lower-dose OTC use of a gliflozin Sodium-Glucose Cotransport 2 inhibitor pharmaceutical composition.

Referring to block 408 of FIG. 4A, the algorithm runs all or a portion of the assessment information set against a second set of assessment filters, where, when a respective filter in the second set of assessment filters is fired, the subject is provided with a warning corresponding to the respective filter. When a respective filter in the second set of filters is fired, the subject is provided with a warning 226 corresponding to the respective filter (e.g., filter warning 228-4 corresponds to filter 222-4). In some embodiments, the warning 226 is provided as a next step, e.g., prior to applying survey results to any subsequent filters, after the corresponding filter is fired. For example, in some embodiments the device provides the subject with a warning prior to proceeding to the next filter, e.g., requiring the subject confirm they have discussed the risk factor underlying the warning with a health care provider and the healthcare provider still recommends taking a gliflozin Sodium-Glucose Cotransport 2 inhibitor pharmaceutical composition. In some embodiments the warning 226 is provided after applying survey results to all subsequent filters. For example, in some embodiments, e.g., when a second category class filter is triggered, the device proceeds to subsequent filters prior to transmitting a warning to the subject, and then transmits all warnings corresponding to filters of the second category class, after survey results have been applied to all subsequent filters.

In some embodiments, the second plurality of assessment filters 222 of the second category class 220 includes some or all of the filters listed in Table 3 (e.g., as illustrated with regards to FIGS. 3B-3G). For example, in some embodiments, the first set of assessment filters includes 2, 3, 4, or all 5 of the filters listed in Table 3.

TABLE 3
Examples of Assessment Filters of the Second Category Class

| Filter | Exemplary Criteria |
|---|---|
| 1b | a urinary tract infection filter 222-1 |
| 2b | a pregnancy filter 222-2 |
| 3b | a bone fracture filter 222-3 |
| 4b | a moderate drug interaction filter 222-4 |
| 5b | an amputation risk filter 222-5 |
| 6b | a hypotension filter 222-6 |

In one embodiment, the second set of assessment filters includes at least filter 1b as provided in Table 3. In another embodiment, the second set of assessment filters includes at least filters 1b and 2b as provided in Table 3. In another embodiment, the second set of assessment filters includes at least filters 1b and 3b-5b as provided in Table 3. In another embodiment, the second set of assessment filters includes at least filters 1b and 5b as provided in Table 3. It is contemplated that, in some embodiments, any one or more of the filters 222 provided in Table 3 will not be included in the second set of assessment filters. For example, in some embodiments, a characteristic associated with a particular survey result will be informative when qualifying a subject for one particular gliflozin Sodium-Glucose Cotransport 2 inhibitor pharmaceutical composition but not for another gliflozin Sodium-Glucose Cotransport 2 inhibitor pharmaceutical composition.

Accordingly, it is contemplated that the second set of assessment filters includes any sub-set of filters 222 provided in Table 3. Likewise, the skilled artisan may know of other filters 222, not provided in Table 3, which may be combined with any subset of the filters 222 provided in Table 3 to form the second set of assessment filters results used in the methods described herein. For brevity, all possible combinations of the filters 222 provided in Table 3 are not specifically delineated here.

In some embodiments, the second set of assessment filters includes a urinary tract infection filter (e.g., urinary tract infection filter 222-1 in FIG. 3 and/or filter 1b in Table 3). The urinary tract infection filter is configured to be fired at least when the assessment information set indicates that the subject has a history of urinary tract infections. In some embodiments, the first urinary problem filter is also fired when the assessment information set indicates that the subject has a history of problems urinating. When urinary tract infection filter is fired, the device transmits a warning corresponding to the urinary tract infection filter and requires the user to acknowledge the warning before authorizing a provision of the gliflozin Sodium-Glucose Cotransport 2 inhibitor pharmaceutical composition. In some embodiments, a urinary tract infection filter is applied as a first filter category class 214 type filter, e.g., when urinary tract infections are contraindications, rather than risk factors, for use of the gliflozin Sodium-Glucose Cotransport 2 inhibitor pharmaceutical composition.

In some embodiments, the second set of assessment filters includes, when the subject is a female, a pregnancy filter (e.g., pregnancy filter 222-2 in FIG. 3 and/or filter 2b in Table 3). The pregnancy filter is configured to be fired at least when the assessment information set indicates the subject is pregnant or breastfeeding. In some embodiments, the pregnancy filter is also configured to be fired when the assessment information set indicates the subject is female and planning on becoming pregnant. When the pregnancy filter is fired, the device transmits a warning corresponding to the pregnancy filter and requires the user to acknowledge the warning before authorizing a provision of the gliflozin Sodium-Glucose Cotransport 2 inhibitor pharmaceutical composition. In some embodiments, a pregnancy filter is applied as a first filter category class 214 type filter, e.g., when pregnancy, breastfeeding, and/or planning to become pregnant are contraindications, rather than risk factors, for use of the gliflozin Sodium-Glucose Cotransport 2 inhibitor pharmaceutical composition.

In some embodiments, the second set of assessment filters includes a bone fracture filter (e.g., bone fracture filter 222-3 in FIG. 3 and/or filter 3b in Table 3). The bone fracture filter is configured to be fired at least when the assessment information set includes an indication that the subject has an increased risk for bone fractures. In some embodiments, a bone fracture filter is implemented when the gliflozin Sodium-Glucose Cotransport 2 inhibitor pharmaceutical composition includes canagliflozin as an active ingredient. When the bone fracture filter is fired, the device transmits a warning corresponding to the bone fracture filter and requires the user to acknowledge the warning before authorizing a provision of the gliflozin Sodium-Glucose Cotransport 2 inhibitor pharmaceutical composition. In some embodiments, a bone fracture filter is applied as a first filter category class 214 type filter, e.g., when an increased risk of bone fracture is a contraindication, rather than a risk factor, for use of the gliflozin Sodium-Glucose Cotransport 2 inhibitor pharmaceutical composition.

In some embodiments, the second set of assessment filters includes a moderate drug interaction filter (e.g., moderate drug interaction filter 222-4 in FIG. 3 and/or filter 4b in Table 3). The moderate drug interaction filter is configured to be fired at least when the assessment information set indicates the subject is taking one or more compositions that interact with the gliflozin Sodium-Glucose Cotransport 2 inhibitor pharmaceutical composition, where the one or more compositions are each associated with a warning, but are not contraindicated, for co-administration with the gliflozin Sodium-Glucose Cotransport 2 inhibitor pharmaceutical composition. These interactions can be pharmacodynamic drug-drug interactions or pharmacokinetic drug-drug interactions. In some embodiments, the moderate drug interaction filter is fired when the assessment information set indicates the subject is taking a medication selected from digoxin, phenytoin, phenobarbital, ritonavir and rifampin. In some embodiments, a moderate drug interaction filter is implemented when the gliflozin Sodium-Glucose Cotransport 2 inhibitor pharmaceutical composition includes canagliflozin as an active ingredient. Additional drugs can be included as part of the moderate drug interaction filter. When the moderate drug interaction filter is fired, the device transmits a warning corresponding to the moderate drug interaction filter and requires the user to acknowledge the warning before authorizing a provision of the gliflozin Sodium-Glucose Cotransport 2 inhibitor pharmaceutical composition. In some embodiments, a moderate drug interaction filter is applied as a first filter category class 214 type filter, e.g., when coadministration of a particular medication is a contraindication, rather than a risk factor, for use of the gliflozin Sodium-Glucose Cotransport 2 inhibitor pharmaceutical composition.

In some embodiments, the second set of assessment filters includes an amputation risk filter (e.g., amputation risk interaction filter 222-5 in FIG. 3 and/or filter 5b in Table 3). The amputation risk filter is configured to be fired at least when the assessment information set includes an indication that the subject has an increased risk for amputation, e.g., lower limb amputations. Non-limiting examples of factors indicating an increased risk of amputation, that can be used as triggering conditions of the filter, include a history of amputation, a history of heart disease, a high risk of heart disease, blocked or narrowed blood vessels, leg neuropathy, and diabetic foot ulcers or sores. In some embodiments, an amputation risk filter is implemented when the gliflozin Sodium-Glucose Cotransport 2 inhibitor pharmaceutical composition includes canagliflozin or ertugliflozin as an active ingredient. When the amputation risk filter is fired, the device transmits a warning corresponding to the bone fracture filter and requires the user to acknowledge the warning before authorizing a provision of the gliflozin Sodium-Glucose Cotransport 2 inhibitor pharmaceutical composition. In some embodiments, an amputation risk filter is applied as a first filter category class 214 type filter, e.g., when an increased risk of amputation is a contraindication, rather than a risk factor, for use of the gliflozin Sodium-Glucose Cotransport 2 inhibitor pharmaceutical composition.

In some embodiments, the second set of assessment filters includes a hypotension filter (e.g., hypotension filter 222-6 and/or filter 6b in Table 3). The hypotension filter is configured to be fired at least when the assessment information set indicates that the subject's systolic blood pressure is less than 90 mm Hg. In other embodiments, the hypotension filter is fired when the assessment information set indicates the subject's systolic blood pressure is less than 75, 76, 77, 78, 79, 80, 81, 82, 83, 84, 85, 86, 87, 88, 89, or 90 mm Hg. In other embodiments, the hypotension filter is fired when the assessment information set indicates the subject's systolic blood pressure is from 75 mm Hg to 95 mm Hg. When the hypotension filter is fired, the device transmits a warning corresponding to the hypotension filter and requires the user to acknowledge the warning before authorizing a provision of the gliflozin Sodium-Glucose Cotransport 2 inhibitor pharmaceutical composition. In some embodiments, a hypotension filter is applied as a first filter category class 214 type filter, e.g., when hypotension is a contraindication, rather than a risk factor, for use of the gliflozin Sodium-Glucose Cotransport 2 inhibitor pharmaceutical composition.

In some embodiments, the warning corresponding to a respective filter in the second set of filters includes a prompt for the subject to indicate whether they have discussed the risk factor underlying the respective filter in the second plurality of filters that was fired with a health care provider (e.g., and that the health care provide still recommended taking a gliflozin Sodium-Glucose Cotransport 2 inhibitor pharmaceutical composition), and acknowledgement is obtained from the subject when the subject indicates that they have discussed the risk factor underlying the respective filter in the second plurality of filters that was fired with a health care provider.

In some embodiments when a respective filter in the second set of assessment filters is fired, a record associated with the firing of the respective filter is stored (e.g., memorializing an adverse event that is required to be reported to a regulatory agency). This record is stored in an adverse event module 242 which includes records of filter firing events associated with a plurality of subjects (e.g., an aggregation of adverse events associated with the gliflozin Sodium-Glucose Cotransport 2 inhibitor pharmaceutical composition across a population of subjects taking the gliflozin Sodium-Glucose Cotransport 2 inhibitor pharmaceutical composition over-the-counter). In some embodiments, an indication of the adverse event is communicated to a third party (e.g., a medical practitioner associated with the subject, a health care provider of the subject, and/or a manufacturer/promoter of the gliflozin Sodium-Glucose Cotransport 2 inhibitor pharmaceutical composition). In some embodiments, the indication is automatically stored in the adverse event module 242 when submitted by a subject.

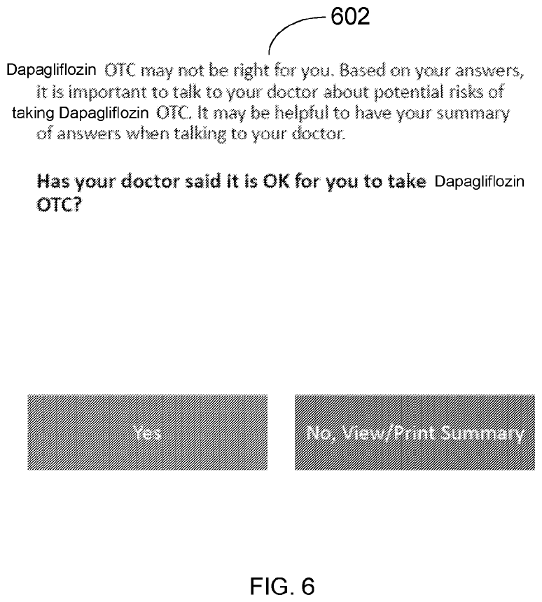

Accordingly, in some embodiments, the algorithm obtains acknowledgment from the subject confirming that the subject has discussed the risk factor associated with each warning issued to the subject by any filter in the second set of filters with a physician (e.g., a licensed medical practitioner), e.g., and the health care practitioner indicated that the subject should take a gliflozin Sodium-Glucose Cotransport 2 inhibitor pharmaceutical composition in view of the underlying risk factor. Accordingly, acknowledgement is obtained from the subject when the subject indicates that they have discussed the risk factor underlying the respective filter in the second plurality of filters that was fired with a health care provider. For example, message 602 in FIG. 6 illustrates a warning that is generic to any fired filters. In some embodiments, the warning is specific to a particular filter (e.g., filter warning 226 in FIG. 2), e.g., communicating to the user why the filter was fired.

In some embodiments, an acknowledgment from the user is verified by the health care practitioner (e.g., the method requires verification in order for authorization of the provision of the gliflozin Sodium-Glucose Cotransport 2 inhibitor pharmaceutical composition), e.g., in order to verify an accuracy of the survey results of the subject. In some embodiments, e.g., when the acknowledgment is verified by the heath care practitioner, the subject is deemed a trusted subject, such that verification of future results is not required.

Referring to blocks 412-424, the algorithm proceeds with a fulfillment process when (a) no filter 216 in the first set of assessment filters has been fired and (b) the subject has acknowledged each warning associated with each filter 222 in the second set of assessment filters that was fired. In some embodiments, the fulfillment process includes storing (414) an indication in a subject profile (e.g., user profile 234) of an initial authorization for the gliflozin Sodium-Glucose Cotransport 2 inhibitor pharmaceutical composition. In some embodiments, the fulfillment process includes communicating (420) an over-the-counter drug facts label for the fulfillment process includes to the subject, and authorizing (422), upon confirmation from the subject that the over-the-counter drug facts label has been received and read, provision of the gliflozin Sodium-Glucose Cotransport 2 inhibitor pharmaceutical composition to the subject.

In some embodiments, the fulfillment process includes storing an indication in a user profile 234 of an initial authorization and/or order date and/or destination for the gliflozin Sodium-Glucose Cotransport 2 inhibitor pharmaceutical composition. The initial authorization/order date is utilized, for example, to verify at least a refill status of a provision of the gliflozin Sodium-Glucose Cotransport 2 inhibitor pharmaceutical composition. The initial authorization/order date is also utilized, for example, to verify at least an elapsed period of time between an initial order and a future re-order. Such verification is required in order to ensure that certain tests (e.g., blood glucose tests) are taken regularly.

In some embodiments, the drug facts label is communicated to the subject in real-time, e.g., within the same user interface as used for the qualification process. In some embodiments, the over-the-counter drug facts label 230 specifies what the gliflozin Sodium-Glucose Cotransport 2 inhibitor pharmaceutical composition is for (e.g., to lower blood sugar, to treat prediabetes, to delay the onset of diabetes, etc.) and any risks associated with taking the gliflozin Sodium-Glucose Cotransport 2 inhibitor pharmaceutical composition (e.g., drug-drug interactions, pharmacokinetic interactions, adverse reactions, etc.) For instance, in some embodiments, the over-the-counter drug facts label 230 specifies that the gliflozin Sodium-Glucose Cotransport 2 inhibitor pharmaceutical composition is to be taken by the subject at a predetermined dosage per day that is from 5 mg to 10 mg per day (block 438). In another example embodiment, the over-the-counter drug facts label 230 specifies that the gliflozin Sodium-Glucose Cotransport 2 inhibitor pharmaceutical composition is to be taken by the subject at a predetermined dosage per day that is 5 mg per day (block 439).

In some embodiments of the aspects disclosed above, the gliflozin Sodium-Glucose Cotransport 2 inhibitor pharmaceutical composition includes dapagliflozin or a pharmaceutically acceptable salt thereof as an active agent. In some embodiments, the provision is for from 2.5 mg to 10 mg dapagliflozin per day. In some embodiments, the provision is for from 2.5 mg to 7.5 mg dapagliflozin per day. In some embodiments, the provision is for 2.5 mg dapagliflozin per day. In some embodiments, the provision is for 5 mg dapagliflozin per day. In some embodiments, the provision is for 7.5 mg dapagliflozin per day. In some embodiments, the provision is for 10 mg dapagliflozin per day.

In some embodiments of the aspects disclosed above, the gliflozin Sodium-Glucose Cotransport 2 inhibitor pharmaceutical composition includes dapagliflozin propanediol as an active agent. In some embodiments, the provision is for from 2.5 mg to 10 mg dapagliflozin propanediol per day. In some embodiments, the provision is for from 2.5 mg to 7.5 mg dapagliflozin propanediol per day. In some embodiments, the provision is for 2.5 mg dapagliflozin propanediol per day. In some embodiments, the provision is for 5 mg dapagliflozin propanediol per day. In some embodiments, the provision is for 7.5 mg dapagliflozin propanediol per day. In some embodiments, the provision is for 10 mg dapagliflozin propanediol per day.

In some embodiments of the aspects disclosed above, upon confirmation from the subject that the over-the-counter drug facts label has been received and read, the subject is authorized for provision of a dosage of from 2.5 mg to 10 mg per day of the gliflozin Sodium-Glucose Cotransport inhibitor pharmaceutical composition.

In some embodiments of the aspects disclosed above, upon confirmation from the subject that the over-the-counter drug facts label has been received and read, the subject is authorized for provision of a dosage of 5 mg per day of the active agent of the gliflozin Sodium-Glucose Cotransport inhibitor pharmaceutical composition.

In some embodiments of the aspects disclosed above, the gliflozin Sodium-Glucose Cotransport inhibitor pharmaceutical composition includes an active agent selected from empagliflozin, canagliflozin, and ertugliflozin.

In some embodiments of the aspects disclosed above, the gliflozin Sodium-Glucose Cotransport inhibitor pharmaceutical composition includes empagliflozin as an active agent and the subject is authorized for provision of a dosage of from 2.5 mg to 50 mg per day. In some embodiments, the subject is authorized for provision of from 2.5 mg to 10 mg per day of empagliflozin. In some embodiments, the subject is authorized for provision of from 2.5 mg to 7.5 mg per day of empagliflozin. In some embodiments, the subject is authorized for provision of from 7.5 mg to 12.5 mg per day of empagliflozin. In some embodiments, the subject is authorized for provision of from 20 mg to 30 mg per day of empagliflozin. In some embodiments, the subject is authorized for provision of 5 mg per day of empagliflozin. In some embodiments, the subject is authorized for provision of 10 mg per day of empagliflozin. In some embodiments, the subject is authorized for provision of 25 mg per day of empagliflozin.

In some embodiments of the aspects disclosed above, the gliflozin Sodium-Glucose Cotransport inhibitor pharmaceutical composition includes canagliflozin as an active agent and the subject is authorized for provision of a dosage of from 25 mg to 600 mg canagliflozin per day. In some embodiments, the subject is authorized for provision of from 25 to 300 mg canagliflozin per day. In some embodiments, the subject is authorized for provision of from 25 to 200 mg canagliflozin per day. In some embodiments, the subject is authorized for provision of from 25 to 100 mg canagliflozin per day. In some embodiments, the subject is authorized for provision of from 25 to 75 mg canagliflozin per day. In some embodiments, the subject is authorized for provision of 25 mg canagliflozin per day. In some embodiments, the subject is authorized for provision of 50 mg canagliflozin per day. In some embodiments, the subject is authorized for provision of 100 mg canagliflozin per day. In some embodiments, the subject is authorized for provision of 150 mg canagliflozin per day. In some embodiments, the subject is authorized for provision of 200 mg canagliflozin per day. In some embodiments, the subject is authorized for provision of 300 mg canagliflozin per day.

In some embodiments of the aspects disclosed above, the gliflozin Sodium-Glucose Cotransport inhibitor pharmaceutical composition includes ertugliflozin as an active agent and the subject is authorized for provision of a dosage of from 1 mg to 30 mg ertugliflozin per day. In some embodiments, the subject is authorized for provision of from 1 to 15 mg ertugliflozin per day. In some embodiments, the subject is authorized for provision of from 1 to 5 mg ertugliflozin per day. In some embodiments, the subject is authorized for provision of 2.5 mg ertugliflozin per day. In some embodiments, the subject is authorized for provision of 5 mg ertugliflozin per day. In some embodiments, the subject is authorized for provision of 10 mg ertugliflozin per day. In some embodiments, the subject is authorized for provision of 15 mg ertugliflozin per day. In some embodiments, the subject is authorized for provision of 20 mg ertugliflozin per day.

In some embodiments, the authorization of providing the gliflozin Sodium-Glucose Cotransport inhibitor pharmaceutical composition to the subject occurs upon confirmation from the subject that the over-the-counter drug facts label 230 has been received and read by the subject. In some embodiments, this authorization includes a destination associated with the subject.

In some embodiments, the fulfillment process further includes storing a destination associated with the subject in the subject profile. In some embodiments, the fulfillment process further includes coordinating shipping of the gliflozin Sodium-Glucose Cotransport inhibitor pharmaceutical composition to a physical address associated with the subject.

In some embodiments, the destination associated with the subject is a physical address including a street address, a Post Office box, a pharmacy associated with the subject, a health care provider associated with the subject, and/or one or more coordinates (e.g., longitude, latitude, elevation). In some embodiments, the provision of the gliflozin Sodium-Glucose Cotransport inhibitor pharmaceutical composition to the subject includes shipping the gliflozin Sodium-Glucose Cotransport inhibitor pharmaceutical composition to the physical address associated with the subject. In some embodiments, the provision of the gliflozin Sodium-Glucose Cotransport inhibitor pharmaceutical composition to the subject includes shipping the gliflozin Sodium-Glucose Cotransport inhibitor pharmaceutical composition to a pharmacy associated and/or a location associated with a health care provider of the subject and/or an office of a medical practitioner associated with the subject.

In some embodiments, the methods described herein further include administering, upon authorization of the provision, the gliflozin Sodium-Glucose Cotransport inhibitor pharmaceutical composition to the subject.

In some embodiments, the administering an over-the-counter gliflozin Sodium-Glucose Cotransport inhibitor pharmaceutical composition to a subject in need thereof is for the purpose of lowering of blood sugar and serves to treat prediabetes and/or delay onset of diabetes in the subject.

In some embodiments, e.g., when the method is terminated without authorization of the gliflozin Sodium-Glucose Cotransport inhibitor pharmaceutical composition, the subject is prevented from attempting to requalify for the gliflozin Sodium-Glucose Cotransport inhibitor pharmaceutical composition for a predetermined period of time, e.g., an hour, 6 hours, 12 hours, 1 day, 2 days, a week, a month, etc. This prevents the subject from abusing the systems and methods of the present disclosure.

### Requalification

An additional goal of the present disclosure is to continue to provide a gliflozin Sodium-Glucose Cotransport inhibitor pharmaceutical composition to a subject over time, e.g., reauthorizing the subject for a provision of the gliflozin Sodium-Glucose Cotransport inhibitor pharmaceutical composition at various time points. Refill authorizations depend on the subject requalifying for the gliflozin Sodium-Glucose Cotransport inhibitor pharmaceutical composition.

Referring to method 450 illustrated in FIG. 4B, in some embodiments, for a human subject who was previously qualified for an over-the-counter provision of a gliflozin Sodium-Glucose Cotransport inhibitor pharmaceutical composition, the method includes receiving (452) a re-order request (e.g., at device 250) from the subject for the gliflozin Sodium-Glucose Cotransport inhibitor pharmaceutical composition.

The method includes obtaining (454) a reassessment information set from the subject (e.g., providing the subject with a reassessment survey 210 to obtain the reassessment information set). In some embodiments, the second plurality of survey results (e.g., the reassessment information set) includes a plurality of survey results selected from the survey results listed in Table 4. In some embodiments, the reassessment survey is configured to obtain a second plurality of survey results. These results are derived from corresponding survey questions (e.g., the device transmits one or more survey questions to the user, prompting a response, and then receives a response to the one or more survey questions back from the subject). In some embodiments, the second plurality of survey results (e.g., as part of the reassessment information set) include some or all of the characteristics listed in Table 4. For example, in some embodiments, the second plurality of survey results includes 1, 2, 3, 4, 5, 6, or all 7 of the characteristics listed in Table 4. In one embodiment, the reassessment survey questions and results include at least characteristics 1-5 as provided in Table 4.

It will be appreciated that the reassessment survey questions 208, 212 and reassessment filters 217, 223 applied to the survey answers (e.g., to the assessment information set) thereof obtained may vary depending upon the gliflozin Sodium-Glucose Cotransport 2 inhibitor pharmaceutical composition being distributed. This is due to differences in the contra-indication profiles of the various the gliflozin Sodium-Glucose Cotransport 2 inhibitor pharmaceutical composition, e.g., due to different drug-drug interactions, routes of drug clearance, etc. of the different gliflozin Sodium-Glucose Cotransport 2 inhibitor pharmaceutical composition.

In some embodiments, the reassessment survey includes questions that elicit responses providing some or all of the characteristics listed in Table 4. In some embodiments, the reassessment survey includes questions corresponding to each of the survey results required for the methods described herein. In other embodiments, the reassessment survey includes questions corresponding to only a subset of the survey results required for the methods described herein. In such embodiments, other survey results required for the methods described herein are acquired through other means (e.g., upon registration/subscription for a service associated with qualifying the subject for over-the-counter medication, from a healthcare provider, from a prior survey, from a database associated with a pharmacy, etc.) For example, in some embodiments, the subject provides a personal medical identification associated with an insurer, a hospital, or other healthcare provider and information about the subject required for the methods described herein, e.g., one or more survey results, is acquired from a preexisting database associated with the personal medical identification (e.g., a last blood sugar measurement determined for the subject).

TABLE 4
Example Reassessment Survey Question Characteristics

| Result | Example Characteristics |
|---|---|
| 1 | when the subject is female, whether the subject is |
| | pregnant or breastfeeding |
| 2 | whether the subject has started taking a medication for |
| | treating diabetes other than the gliflozin Sodium-Glucose |
| | Cotransport 2 inhibitor pharmaceutical composition since |
| | receiving their last over-the-counter provision of the |
| | gliflozin Sodium-Glucose Cotransport 2 inhibitor |
| | pharmaceutical composition |
| 3 | an indication of whether the subject has developed kidney |
| | disease since receiving their last over-the-counter |
| | provision of the gliflozin Sodium-Glucose Cotransport 2 |
| | inhibitor pharmaceutical composition |
| 4 | an indication of whether the subject has experienced a |
| | urinary tract infection since receiving their last |
| | over-the-counter provision of the gliflozin Sodium-Glucose |
| | Cotransport 2 inhibitor pharmaceutical composition |
| 5 | an indication of whether the subject has experienced a |
| | bacterial infection since receiving their last |
| | over-the-counter provision of the gliflozin Sodium-Glucose |
| | Cotransport 2 inhibitor pharmaceutical composition |
| 6 | An indication of the subject's LDL-C level |
| 7 | An indication of the subject's blood glucose level |
| 8 | An indication of the subject's body mass index (BMI) |

It is contemplated that, in some embodiments, any one or more of the survey questions provided in Table 4 will not be included in the reassessment survey (e.g., will not be used for the reassessment). For example, in some embodiments, a characteristic associated with a particular survey questions will be informative when qualifying a subject for one particular gliflozin Sodium-Glucose Cotransport 2 inhibitor pharmaceutical composition but not for another gliflozin Sodium-Glucose Cotransport 2 inhibitor pharmaceutical composition. The skilled artisan will recognize that different gliflozin Sodium-Glucose Cotransport 2 inhibitor pharmaceutical composition carry different risk and drug interaction profiles. Accordingly, survey information required for qualifying a subject for access to one gliflozin Sodium-Glucose Cotransport 2 inhibitor pharmaceutical composition with a known adverse drug interaction may not be necessary for qualifying the same subject for access to a second gliflozin Sodium-Glucose Cotransport 2 inhibitor pharmaceutical composition.

Accordingly, it is contemplated that the reassessment survey questions elicit responses to any sub-set of survey results provided in Table 4. For brevity, all possible combinations of the characteristics provided in Table 4 are not specifically delineated here. However, the skilled artisan will easily be able to envision any particular subset of survey questions designed to elicit responses to any subset of characteristics provided in Table 4. Likewise, the skilled artisan may know of other survey questions, not provided in Table 4, that may be combined with any subset of the survey questions provided in Table 4 to form the reassessment survey questions used in the methods described herein.

### Reassessment Filters

All or a portion the reassessment survey results are run against a first set of reassessment filters of the first category class. When a respective filter in the first set of reassessment filters is fired (e.g., when a reassessment survey result indicates that a triggering condition 218 has been met), the subject is deemed not qualified for the gliflozin Sodium-Glucose Cotransport 2 inhibitor pharmaceutical composition and the method is terminated without reauthorizing the subject for the gliflozin Sodium-Glucose Cotransport 2 inhibitor pharmaceutical composition. Specific filters in the first set of reassessment filters and their exemplary triggering conditions that cause the corresponding filter to fire are detailed.

In some embodiments, the first set of reassessment filters of the first category class includes some or all of the filters listed in Table 5. For example, in some embodiments, the first set of reassessment filters includes 1, 2, 3, 4, 5, 6, of all 7 of the filters listed in Table 5 (e.g., as also illustrated in FIG. 3).

TABLE 5
Examples of Reassessment Filters of the First Category Class

| Filter | Example Criteria |
|---|---|
| 1c | a pregnancy filter 217-1 |
| 2c | a diabetes medication filter 217-2 |
| 3c | a kidney disease filter 217-3 |
| 4c | a urinary tract infection filter 217-4 |
| 5c | a bacterial infection filter 217-5 |
| 6c | an LDL-C filter 217-6 |
| 7c | a blood glucose filter 217-7 |
| 8c | a body mass index filter 217-8 |

In one embodiment, the first set of reassessment filters includes at least filters 1c-5c as provided in Table 5. In one embodiment, the first set of reassessment filters includes at least filters 1c-5c and 7c as provided in Table 5. In one embodiment, the first set of reassessment filters includes at least filters 1c-6c as provided in Table 5. In one embodiment, the first set of reassessment filters includes at least filters 1c-7c as provided in Table 5. It is contemplated that, in some embodiments, any one or more of the filters 217 provided in Table 5 will not be included in the first set of reassessment filters. For example, in some embodiments, a characteristic associated with a particular survey result will be informative when qualifying a subject for one particular gliflozin Sodium-Glucose Cotransport 2 inhibitor pharmaceutical composition but not for another gliflozin Sodium-Glucose Cotransport 2 inhibitor pharmaceutical composition.

Accordingly, it is contemplated that the first set of reassessment filters includes any sub-set of filters 217 provided in Table 5. Likewise, the skilled artisan may know of other filters 217, not provided in Table 5, which may be combined with any subset of the filters 217 provided in Table 5 to form the first set of assessment filters results used in the methods described herein. For brevity, all possible combinations of the filters 217 provided in Table 5 are not specifically delineated here.

In some embodiments, the first set of reassessment filters includes, when the subject is a female, a pregnancy filter (e.g., pregnancy filter 217-1 in FIG. 3 and/or filter 1c in Table 5). The pregnancy filter is configured to be fired at least when the reassessment information set indicates the subject is pregnant or breastfeeding. In some embodiments, the pregnancy filter is also configured to be fired when the reassessment information set indicates the subject is female and planning on becoming pregnant. When the pregnancy filter is fired, the subject is not permitted to obtain the gliflozin Sodium-Glucose Cotransport 2 inhibitor pharmaceutical composition over-the-counter (e.g., the method is terminated without authorizing provision of the gliflozin Sodium-Glucose Cotransport 2 inhibitor pharmaceutical composition). In some embodiments, a pregnancy filter is applied as a second filter category class 221 type filter, e.g., when pregnancy, breastfeeding, and/or planning to become pregnant are risk factors, rather than contraindications, for use of the gliflozin Sodium-Glucose Cotransport 2 inhibitor pharmaceutical composition.

In some embodiments, the first set of reassessment filters includes a diabetes medication filter (e.g., diabetes medication filter 217-2 in FIG. 3 and/or filter 2c in Table 5). The diabetes medication filter is configured to be fired at least when the reassessment information set indicates the subject has started taking a medication for treating diabetes other than the gliflozin Sodium-Glucose Cotransport 2 inhibitor pharmaceutical composition since receiving their last over-the-counter provision of the gliflozin Sodium-Glucose Cotransport 2 inhibitor pharmaceutical composition, regardless of whether the composition was prescribed specifically for treatment of diabetes or not. Non-limiting examples of diabetes medications that may trigger a diabetes medication filter include a gliflozin Sodium-Glucose Cotransport 2 inhibitor pharmaceutical composition, insulin or an analog thereof, an amylinomimetic drug, an alpha-glucosidase inhibitor, a biguanide (e.g., metformin), a dopamine agonist, a dipeptidyl peptidase-4 (DPP-4) inhibitor, a glucagon-like peptide-1 receptor agonist (GLP-1 receptor agonist), a meglitinide, a sulfonylurea, and a thiazolidinedione. If the diabetes medication filter is fired, the subject is not permitted to obtain the gliflozin Sodium-Glucose Cotransport 2 inhibitor pharmaceutical composition over-the-counter (e.g., the method is terminated without authorizing provision of the gliflozin Sodium-Glucose Cotransport 2 inhibitor pharmaceutical composition to the subject). In some embodiments, a diabetes medication filter is applied as a second filter category class 221 type filter, e.g., when co-administration of a diabetes medication is a risk factor, rather than a contraindication, for use of the gliflozin Sodium-Glucose Cotransport 2 inhibitor pharmaceutical composition over-the-counter.

In some embodiments, the first set of reassessment filters includes a kidney disease filter (e.g., kidney disease filter 217-3 in FIG. 3 and/or filter 3c in Table 5). The kidney disease filter is configured to be fired at least when the reassessment information set indicates that the subject has developed kidney disease since receiving their last over-the-counter provision of the gliflozin Sodium-Glucose Cotransport 2 inhibitor pharmaceutical composition. In some embodiments, the kidney disease filter is configured to be fired when the assessment information set includes an indication that the subject is currently receiving kidney dialysis. Accordingly, in some embodiments, a reassessment survey administered to the subject asks one or both of (i) whether the subject has been diagnosed with kidney disease, and (ii) whether the subject is receiving kidney dialysis. In some embodiments, separate filters are implemented for a kidney disease diagnosis and receipt of kidney dialysis, e.g., both of which are of the first category class. In some embodiments, indications of a kidney problem include a reduction in food or liquid intake, vomiting, diarrhea, or dehydration. If the kidney disease filter is fired, the subject is not permitted to obtain the gliflozin Sodium-Glucose Cotransport 2 inhibitor pharmaceutical composition pharmaceutical composition over-the-counter (e.g., the method is terminated without authorizing provision of the gliflozin Sodium-Glucose Cotransport 2 inhibitor pharmaceutical composition to the subject). In some embodiments, one or more kidney disease filter is applied as a second filter category class 221 type filter, e.g., when kidney disease is a risk factor, rather than a contraindication, for use of the gliflozin Sodium-Glucose Cotransport 2 inhibitor pharmaceutical composition over-the-counter.

In some embodiments, the first set of reassessment filters includes a urinary tract infection filter (e.g., urinary tract infection filter 217-4 in FIG. 3 and/or filter 4c in Table 5). The urinary tract infection filter is configured to be fired at least when the reassessment information set indicates that the subject has experienced symptoms of a urinary tract infection since receiving their last over-the-counter provision of the gliflozin Sodium-Glucose Cotransport 2 inhibitor pharmaceutical composition. In some embodiments, the urinary tract infection filter is fired when the reassessment information set indicates that the subject has experienced problems urinating, burning feeling when passing urine, an increased need to urinate, a need to urinate immediately after ingesting liquids, pain in the lower stomach, or blood in the subject's urine, since receiving their last over-the-counter provision of the gliflozin Sodium-Glucose Cotransport 2 inhibitor pharmaceutical composition. If the urinary tract infection filter is fired, the subject is not permitted to obtain the gliflozin Sodium-Glucose Cotransport 2 inhibitor pharmaceutical composition pharmaceutical composition over-the-counter (e.g., the method is terminated without authorizing provision of the gliflozin Sodium-Glucose Cotransport 2 inhibitor pharmaceutical composition to the subject). In some embodiments, a urinary tract infection filter is applied as a second filter category class 221 type filter, e.g., when urinary tract infections are a risk factor, rather than a contraindication, for use of the gliflozin Sodium-Glucose Cotransport 2 inhibitor pharmaceutical composition over-the-counter.

In some embodiments, the first set of reassessment filters includes a bacterial infection filter (e.g., bacterial infection filter 217-5 in FIG. 3 and/or filter 5c in Table 5). The bacterial infection filter is configured to be fired at least when the reassessment information set indicates that the subject has experienced symptoms (e.g., unexplained symptoms) of a bacterial infection since receiving their last over-the-counter provision of the gliflozin Sodium-Glucose Cotransport 2 inhibitor pharmaceutical composition. In some embodiments, the bacterial infection filter is fired when the reassessment information set indicates that the subject has experienced pain, tenderness, swelling, or redness around the anus and genitals with accompanying fever or weakness since receiving their last over-the-counter provision of the gliflozin Sodium-Glucose Cotransport 2 inhibitor pharmaceutical composition. If the bacterial infection filter is fired, the subject is not permitted to obtain the gliflozin Sodium-Glucose Cotransport 2 inhibitor pharmaceutical composition pharmaceutical composition over-the-counter (e.g., the method is terminated without authorizing provision of the gliflozin Sodium-Glucose Cotransport 2 inhibitor pharmaceutical composition to the subject). In some embodiments, a bacterial infection filter is applied as a second filter category class 221 type filter, e.g., when a bacterial infection is a risk factor, rather than a contraindication, for use of the gliflozin Sodium-Glucose Cotransport 2 inhibitor pharmaceutical composition over-the-counter.

In some embodiments, the first set of reassessment filters includes an LDL-C filter (e.g., LDL-C filter 217-6 in FIG. 3 and/or filter 6c in Table 5). The LDL-C filter is configured to be fired at least when the reassessment information set indicates the subject's low-density lipoprotein cholesterol (LDL-C) level has increased since receiving their last over-the-counter provision of the gliflozin Sodium-Glucose Cotransport 2 inhibitor pharmaceutical composition. In some embodiments, the increase is at least a 10% increase, at least 15% increase, at least a 20% increase, at least a 25% increase, at least a 30% increase, at least a 40% increase, at least a 50% increase, or more. In some embodiments, an LDL-C filter is implemented when the gliflozin Sodium-Glucose Cotransport 2 inhibitor pharmaceutical composition includes empagliflozin or ertugliflozin as an active ingredient. If the LDL-C filter is fired, the subject is not permitted to obtain the gliflozin Sodium-Glucose Cotransport 2 inhibitor pharmaceutical composition pharmaceutical composition over-the-counter (e.g., the method is terminated without authorizing provision of the gliflozin Sodium-Glucose Cotransport 2 inhibitor pharmaceutical composition to the subject). In some embodiments, an LDL-C filter is applied as a second filter category class 221 type filter, e.g., when an increase in LDL-C level is a risk factor, rather than a contraindication, for use of the gliflozin Sodium-Glucose Cotransport 2 inhibitor pharmaceutical composition over-the-counter.

In some embodiments, the first set of reassessment filters includes a blood glucose (or blood sugar) filter (e.g., blood glucose filter 217-7 in FIG. 3 and/or filter 7c in Table 5). In some embodiments, the blood glucose filter is fired when the first plurality of survey results indicates that the subject has a blood glucose level that is above a ceiling blood glucose level (e.g., corresponding to a blood glucose level that is indicative that the subject has developed diabetes). If the blood glucose filter is fired, the subject is not permitted to obtain the gliflozin Sodium-Glucose Cotransport 2 inhibitor pharmaceutical composition pharmaceutical composition over-the-counter (e.g., the method is terminated without authorizing provision of the gliflozin Sodium-Glucose Cotransport 2 inhibitor pharmaceutical composition to the subject). In some embodiments, one or more blood glucose filter is applied as a second filter category class 223 type filter, e.g., when a high blood glucose level is a risk factor, rather than a contraindication, for use of the gliflozin Sodium-Glucose Cotransport 2 inhibitor pharmaceutical composition over-the-counter.

In some embodiments, the ceiling blood sugar level used in the blood glucose filter is 6.4% glycated hemoglobin (HbA1c), or an equivalent thereof (e.g., an equivalent eAG level). In other embodiments, the ceiling blood sugar level used in the blood glucose filter is from 6.1% HbA1c to 6.7% HbA1c, e.g., 6.1% HbA1c, 6.2% HbA1c, 6.3% HbA1c, 6.4% HbA1c, 6.5% HbA1c, 6.6% HbA1c, or 6.7% HbA1c. In other embodiments, the ceiling blood sugar level used in the blood glucose filter is from 6.2% HbA1c to 6.6% HbA1c. In other embodiments, the first baseline blood glucose level used in the blood glucose filter is from 6.3% HbA1c to 6.5% HbA1c.

In some embodiments, the ceiling blood sugar level used in the blood glucose filter is 125 mg/dL fasting glucose. In other embodiments, the ceiling blood sugar level used in the blood glucose filter is from 112.5 mg/dL to 137.5 mg/dL fasting glucose, e.g., 112.5 mg/dL, 115 mg/dL, 120 mg/dL, 125 mg/dL, 130 mg/dL, 135 mg/dL, or 137.5 mg/dL fasting glucose. In other embodiments, the ceiling blood sugar level used in the blood glucose filter is from 115 mg/dL to 135 mg/dL fasting glucose. In other embodiments, the ceiling blood glucose level used in the blood glucose filter is from 120 mg/dL to 130 mg/dL fasting glucose.

In some embodiments, the first set of reassessment filters includes a body mass index (BMI) filter (e.g., body mass index filter 8c in Table 5). The BMI filter is configured to be fired at least when the reassessment information set indicates the BMI of the subject fails to satisfy a BMI threshold for receiving the gliflozin Sodium-Glucose Cotransport 2 inhibitor pharmaceutical composition. In some embodiments, the BMI filter is fired when the reassessment information set indicates the subject's BMI is less than 25 kg/m². In other embodiments, the BMI filter is fired when the reassessment information set indicates the subject's BMI is less than 20 kg/m², 21 kg/m², 22 kg/m², 23 kg/m², 24 kg/m², 26 kg/m², 27 kg/m², 28 kg/m², 29 kg/m², 30 kg/m², 31 kg/m², 32 kg/m², 33 kg/m², 34 kg/m², or 35 kg/m². In some embodiments, the BMI threshold is from 22.5 to 27.5. In other embodiments, the BMI threshold is from 20 kg/m² to 22.5 kg/m², from 20 kg/m² to 25 kg/m², from 20 kg/m² to 27.5 kg/m², from 20 kg/m² to 30 kg/m², from 22.5 kg/m² to 25 kg/m², from 22.5 kg/m² to 30 kg/m², from 22.5 kg/m² to 35 kg/m², from 25 kg/m² to 27.5 kg/m², from 25 kg/m² to 30 kg/m², or from 27.5 kg/m² to 30 kg/m². If the BMI filter is fired, the subject is not permitted to obtain the gliflozin Sodium-Glucose Cotransport 2 inhibitor pharmaceutical composition over-the-counter (e.g., the method is terminated without authorizing provision of the gliflozin Sodium-Glucose Cotransport 2 inhibitor pharmaceutical composition to the subject). In some embodiments, a BMI filter is applied as a second filter category class 220 type filter, e.g., when a particular range of subject BMI is a risk factor, rather than a contraindication, for use of the gliflozin Sodium-Glucose Cotransport 2 inhibitor pharmaceutical composition. In some embodiments, the reassessment information set includes the height and weight of the subject, and the subject's BMI is determined therefrom. In some embodiments, the reassessment information set includes the BMI of the subject. In some embodiments, the system displays a table correlating subject height and weight with BMI. In some embodiments, BMI is calculated differently based upon whether the subject is male or female.

In some embodiments, the method also includes running all or a portion of the reassessment survey results against a second plurality of reassessment filters of the second category class 220-2. When a respective filter in the second plurality of reassessment filters is fired, the subject is provided with a warning corresponding to the respective filter. In some embodiments, the warning is provided as a next step, e.g., prior to applying survey results to any subsequent filters, after the corresponding filter is fired. In some embodiments the warning is provided after applying survey results to all subsequent filters.

In some embodiments the method also includes obtaining acknowledgment from the subject for each warning issued to the subject by any filter in the second plurality of reassessment filters. As described with respect to the warnings issued in conjunction with the second plurality of filters of the second category class, in some embodiments, the warning includes a prompt for the subject to indicate whether they have discussed the risk factor underlying the respective filter in the second plurality of filters that was fired with a health care practitioner (e.g., a licensed medical practitioner), e.g., and the health care practitioner indicated that the subject should take a gliflozin Sodium-Glucose Cotransport 2 inhibitor pharmaceutical composition in view of the underlying risk factor. Accordingly, acknowledgement is obtained from the subject when the subject indicates that they have discussed the risk factor underlying the respective filter in the second plurality of reassessment filters that was fired with a health care provider.

In some embodiments of the aspects disclosed above, the gliflozin Sodium-Glucose Cotransport 2 inhibitor pharmaceutical composition includes dapagliflozin or a pharmaceutically acceptable salt thereof as an active agent. In some embodiments, the provision is for from 2.5 mg to 10 mg dapagliflozin per day. In some embodiments, the provision is for from 2.5 mg to 7.5 mg dapagliflozin per day. In some embodiments, the provision is for 2.5 mg dapagliflozin per day. In some embodiments, the provision is for 5 mg dapagliflozin per day. In some embodiments, the provision is for 7.5 mg dapagliflozin per day. In some embodiments, the provision is for 10 mg dapagliflozin per day.

In some embodiments of the aspects disclosed above, the gliflozin Sodium-Glucose Cotransport 2 inhibitor pharmaceutical composition includes dapagliflozin propanediol as an active agent. In some embodiments, the provision is for from 2.5 mg to 10 mg dapagliflozin propanediol per day. In some embodiments, the provision is for from 2.5 mg to 7.5 mg dapagliflozin propanediol per day. In some embodiments, the provision is for 2.5 mg dapagliflozin propanediol per day. In some embodiments, the provision is for 5 mg dapagliflozin propanediol per day. In some embodiments, the provision is for 7.5 mg dapagliflozin propanediol per day. In some embodiments, the provision is for 10 mg dapagliflozin propanediol per day.

In some embodiments of the aspects disclosed above, upon confirmation from the subject that the over-the-counter drug facts label has been received and read, the subject is authorized for provision of a dosage of from 2.5 mg to 10 mg per day of the gliflozin Sodium-Glucose Cotransport inhibitor pharmaceutical composition.

In some embodiments of the aspects disclosed above, upon confirmation from the subject that the over-the-counter drug facts label has been received and read, the subject is authorized for provision of a dosage of 5 mg per day of the active agent of the gliflozin Sodium-Glucose Cotransport inhibitor pharmaceutical composition.

In some embodiments of the aspects disclosed above, the gliflozin Sodium-Glucose Cotransport inhibitor pharmaceutical composition includes an active agent selected from empagliflozin, canagliflozin, and ertugliflozin.

In some embodiments of the aspects disclosed above, the gliflozin Sodium-Glucose Cotransport inhibitor pharmaceutical composition includes empagliflozin as an active agent and the subject is authorized for provision of a dosage of from 2.5 mg to 50 mg per day. In some embodiments, the subject is authorized for provision of from 2.5 mg to 10 mg per day of empagliflozin. In some embodiments, the subject is authorized for provision of from 2.5 mg to 7.5 mg per day of empagliflozin. In some embodiments, the subject is authorized for provision of from 7.5 mg to 12.5 mg per day of empagliflozin. In some embodiments, the subject is authorized for provision of from 20 mg to 30 mg per day of empagliflozin. In some embodiments, the subject is authorized for provision of 5 mg per day of empagliflozin. In some embodiments, the subject is authorized for provision of 10 mg per day of empagliflozin. In some embodiments, the subject is authorized for provision of 25 mg per day of empagliflozin.

In some embodiments of the aspects disclosed above, the gliflozin Sodium-Glucose Cotransport inhibitor pharmaceutical composition includes canagliflozin as an active agent and the subject is authorized for provision of a dosage of from 25 mg to 600 mg canagliflozin per day. In some embodiments, the subject is authorized for provision of from 25 to 300 mg canagliflozin per day. In some embodiments, the subject is authorized for provision of from 25 to 200 mg canagliflozin per day. In some embodiments, the subject is authorized for provision of from 25 to 100 mg canagliflozin per day. In some embodiments, the subject is authorized for provision of from 25 to 75 mg canagliflozin per day. In some embodiments, the subject is authorized for provision of 25 mg canagliflozin per day. In some embodiments, the subject is authorized for provision of 50 mg canagliflozin per day. In some embodiments, the subject is authorized for provision of 100 mg canagliflozin per day. In some embodiments, the subject is authorized for provision of 150 mg canagliflozin per day. In some embodiments, the subject is authorized for provision of 200 mg canagliflozin per day. In some embodiments, the subject is authorized for provision of 300 mg canagliflozin per day.

In some embodiments of the aspects disclosed above, the gliflozin Sodium-Glucose Cotransport inhibitor pharmaceutical composition includes ertugliflozin as an active agent and the subject is authorized for provision of a dosage of from 1 mg to 30 mg ertugliflozin per day. In some embodiments, the subject is authorized for provision of from 1 to 15 mg ertugliflozin per day. In some embodiments, the subject is authorized for provision of from 1 to 5 mg ertugliflozin per day. In some embodiments, the subject is authorized for provision of 2.5 mg ertugliflozin per day. In some embodiments, the subject is authorized for provision of 5 mg ertugliflozin per day. In some embodiments, the subject is authorized for provision of 10 mg ertugliflozin per day. In some embodiments, the subject is authorized for provision of 15 mg ertugliflozin per day. In some embodiments, the subject is authorized for provision of 20 mg ertugliflozin per day.

### Example Implementations

FIG. 7 illustrates an example method (700) (e.g., performed at an electronic device) for qualifying a subject for an over-the-counter gliflozin Sodium-Glucose Cotransport 2 inhibitor pharmaceutical composition. In some embodiments, the method of FIG. 7 is utilized when the subject has not been previously qualified for the medication. In some embodiments, the method of FIG. 7 is utilized when the subject was previously qualified for the gliflozin Sodium-Glucose Cotransport 2 inhibitor pharmaceutical composition but a predetermined period of time elapsed since the previous qualification occurred (e.g., the most recent qualification of the subject was greater than one year ago).

Referring to FIG. 7, the device prompts (702) the user to confirm that they know their blood sugar levels (e.g., because the subject must know their blood sugar level values in order to complete the qualification process). If the subject indicates they do not know their blood sugar level, the process terminates 701 without authorizing provision of the gliflozin Sodium-Glucose Cotransport 2 inhibitor pharmaceutical agent, and optionally transmits advice to the user to return later, e.g., once they know their blood sugar levels. In some embodiments, the device does not prompt the user to confirm they know their numbers, but includes a selection for indicating they don't know a value when asking the subject for a particular value. If the subject indicates they know their blood sugar levels, the process continues.

Once the subject has acknowledged they have the requisite knowledge of their blood sugar levels, the device prompts (704) the subject to acknowledge a privacy notice. Since the present disclosure requires the subject to know and input sensitive medical information (e.g., information only the subject and a medical practitioner have access to), privacy of this information is important.

Once the subject has acknowledged they have the requisite privacy for continuing, the device prompts the subject to indicate their age and then applies (706) the answer received from the subject to an age filter. When the age filter is fired (e.g., when the answer indicates the subject is less than eighteen years old), the device terminates (703) the qualification process without authorizing provision of the gliflozin Sodium-Glucose Cotransport 2 inhibitor pharmaceutical agent and, optionally, transmits advice to the user as to why they should not take the gliflozin Sodium-Glucose Cotransport 2 inhibitor pharmaceutical agent and/or to return once they have obtained an age at which it would be appropriate to take a gliflozin Sodium-Glucose Cotransport 2 inhibitor pharmaceutical agent.

When the age filter is not fired, the device proceeds with the qualification process, prompting the subject to provide information about their gender and then applies (708) the answer received from the subject to a gender filter. When the gender filter is fired (e.g., when the answer indicates the subject was identified as male at birth), the device proceeds with the qualification process, prompting the subject to provide information about whether they have diabetes.

When the gender filter is not fired (e.g., when the answer indicates the subject was identified as female at birth), the device proceeds with the qualification process, prompting the subject to provide information about their pregnancy status and then applies (710) the answer received from the subject to a pregnancy filter. When the pregnancy filter is fired (e.g., when the answer indicates the subject is pregnant, breastfeeding, or planning to become pregnant), the device terminates (703) the qualification process without authorizing provision of the gliflozin Sodium-Glucose Cotransport 2 inhibitor pharmaceutical agent and, optionally, transmits advice to the user as to why they should not take the gliflozin Sodium-Glucose Cotransport 2 inhibitor pharmaceutical agent.

When the pregnancy filter is not fired or when the gender filter is fired, the device proceeds with the qualification process, prompting the subject to indicate whether they have diabetes and then applies (712) the answer received from the subject to a diabetes filter. When the diabetes filter is fired (e.g., when the answer indicates the subject has type 1 or type 2 diabetes), the device terminates (703) the qualification process without authorizing provision of the gliflozin Sodium-Glucose Cotransport 2 inhibitor pharmaceutical agent and, optionally, transmits advice to the user as to why they should not take the gliflozin Sodium-Glucose Cotransport 2 inhibitor pharmaceutical agent.

When the diabetes filter is not fired, the device proceeds with the qualification process, prompting the subject to indicate whether the subject is taking a diabetes medication and then applies (714) the answer received from the subject to a diabetes medication filter. When the diabetes medication filter is fired (e.g., when the answer indicates the subject has been taking a diabetes medication), the device terminates (703) the qualification process without authorizing provision of the gliflozin Sodium-Glucose Cotransport 2 inhibitor pharmaceutical agent and, optionally, transmits advice to the user as to why they should not take the gliflozin Sodium-Glucose Cotransport 2 inhibitor pharmaceutical agent.

When the diabetes medication filter is not fired, the device proceeds with the qualification process, prompting the subject to indicate whether they have kidney disease and applies (716) the answered received from the subject to a kidney disease filter. When the kidney disease filter is fired (e.g., when the answer to the prompt indicates the subject has kidney disease), the device terminates (703) the qualification process without authorizing provision of the gliflozin Sodium-Glucose Cotransport 2 inhibitor pharmaceutical agent and, optionally, transmits advice to the user as to why they should not take the gliflozin Sodium-Glucose Cotransport 2 inhibitor pharmaceutical agent.

When the kidney disease filter is not fired, the device proceeds with the qualification process, prompting the subject to indicate whether the subject has knowledge of a first blood sugar level (e.g., an HbA1c blood sugar level of the subject) and applies (718) the answer received from the subject to a first blood sugar filter. When the first blood sugar filter is fired (e.g., when the answer to the prompt indicates the subject knows their HbA1c level), the device proceeds with the qualification process, prompting the subject to provide information about their first blood sugar level and applies (720) the answer received from the subject to a second blood sugar filter. When the second blood sugar filter is not fired (e.g., when the answer indicates the subject has a first blood sugar level that is either below a first floor blood sugar level or above a first ceiling blood sugar level), the process terminates (701) without authorizing provision of the gliflozin Sodium-Glucose Cotransport 2 inhibitor pharmaceutical agent and, optionally, transmits (705) advice to the user to return later when their blood sugar level is within the floor and ceiling levels.

When the first blood sugar filter is not fired, the device proceeds with the qualification process, prompting the subject to provide information about their second blood sugar level (e.g., fasting glucose blood sugar level) and then applies (722) the answer received from the subject to a third blood sugar filter. When the third blood sugar filter is not fired (e.g., when the answer indicates the subject has a second blood sugar level that is either below a second floor blood sugar level or above a second ceiling blood sugar level), the process terminates (701) without authorizing provision of the gliflozin Sodium-Glucose Cotransport 2 inhibitor pharmaceutical agent and, optionally, transmits (705) advice to the user to return later when their blood sugar level is within the floor and ceiling levels.

When the second blood sugar filter or the third blood sugar filter is fired, the device proceeds with the qualification process, prompting the subject to indicate whether they have a urinary problem and then applies (724) the answer received from the subject to a urinary problem filter. When the urinary problem filter is fired (e.g., when the answer indicates the subject has a urinary problem), an override procedure (711-1) is initiated (e.g., the device creates a record indicating that the user must confirm they have discussed taking a gliflozin Sodium-Glucose Cotransport 2 inhibitor pharmaceutical composition with a health care professional).

The device proceeds with the qualification process, determining (734) whether the override procedure has been triggered (e.g., by firing of any one of the urinary problem filter). If the override procedure has been triggered, the device prompts (717) the user to confirm that they have spoken with a medical professional about taking a gliflozin Sodium-Glucose Cotransport 2 inhibitor pharmaceutical composition (e.g., in view of the underlying risk factor that triggered the urinary problem filter) and the medical professional recommended taking the gliflozin Sodium-Glucose Cotransport 2 inhibitor pharmaceutical composition. If the user's responses indicate they have not spoken with a medical professional or the medical professional did not recommend taking the gliflozin Sodium-Glucose Cotransport 2 inhibitor pharmaceutical composition, the device terminates (705) the process and, optionally, transmits advice for the subject to consult a medical professional.

If the override procedure was not triggered, or the override procedure was triggered and the subject's response (717) indicated that a medical professional recommended they take a gliflozin Sodium-Glucose Cotransport 2 inhibitor pharmaceutical composition (e.g., in view of the underlying risk factor triggering the override procedure), the device proceeds with the qualification process, prompting (736) the subject to confirm their answers. If the user confirms their answers, the device transmits (738) a drug facts label for the gliflozin Sodium-Glucose Cotransport 2 inhibitor pharmaceutical composition and prompts the user to read the drug facts label. If the subject confirms they have read the drug facts label (e.g., the subject scrolls through the drug facts label), the device proceeds to authorize (740) purchase of the gliflozin Sodium-Glucose Cotransport 2 inhibitor pharmaceutical composition.

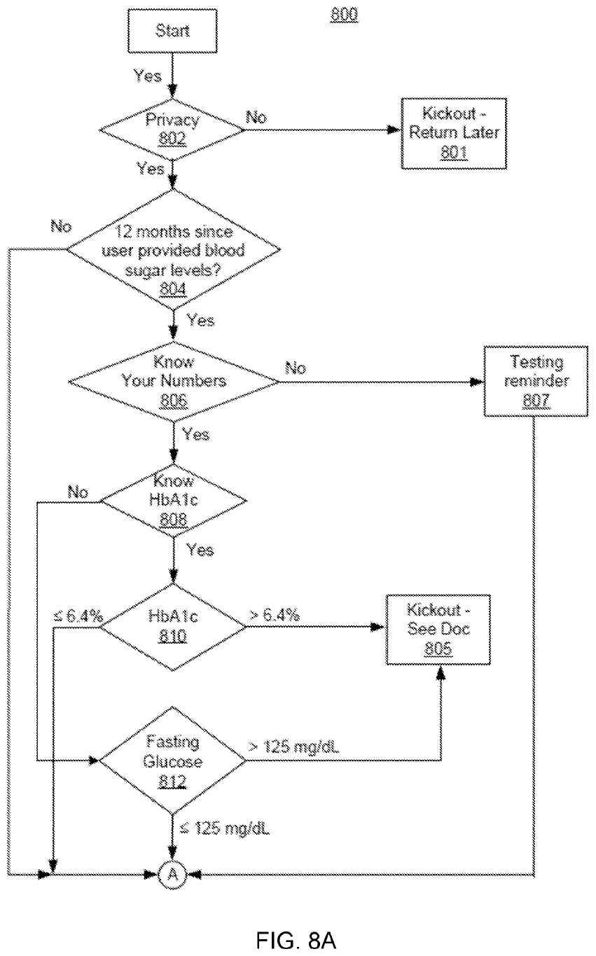

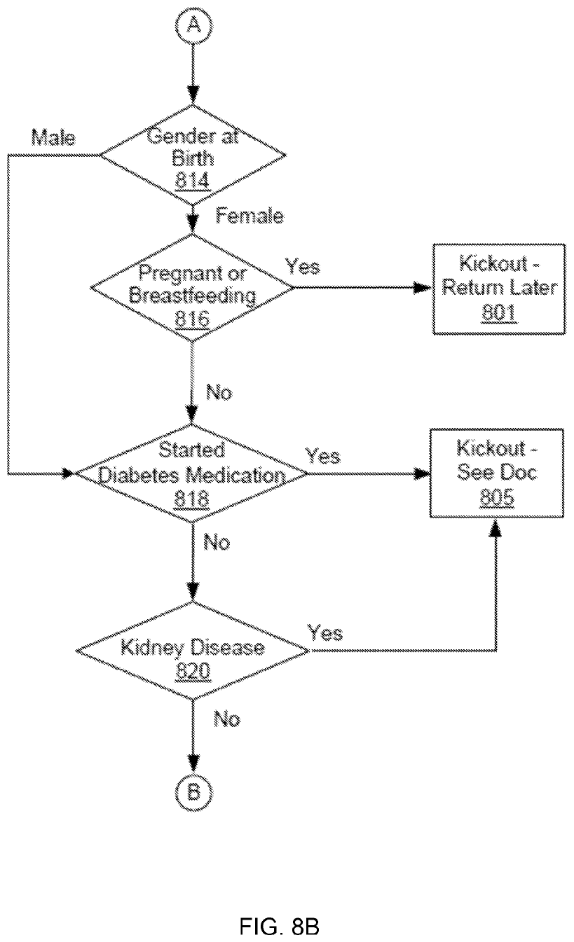

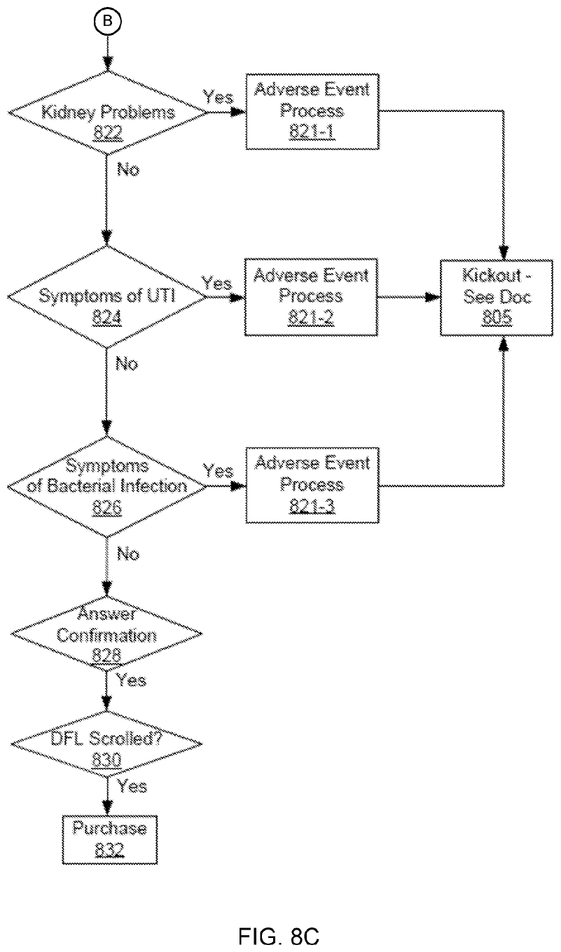

FIG. 8 illustrates an example method (800) for qualifying a subject for a refill of an over-the-counter gliflozin Sodium-Glucose Cotransport 2 inhibitor pharmaceutical composition (e.g., following a prescription from a medical professional or initial qualification by a method described herein). Referring to FIG. 8, the device prompts (802) the subject to acknowledge a privacy notice. Once the subject has acknowledged they have the requisite privacy for continuing, the device determines (804) if the user must confirm that they know their blood sugar levels (e.g., because the subject must know their recent blood sugar levels in order to complete the re-qualification process). If the device determines a blood sugar level has been checked within a predetermined period of time (e.g., within twelve months of an initial order), the device proceeds with the qualification process, prompting the subject to provide information about their gender. When a new blood sugar input is required (e.g., when the subject's profile does not include a record of the subject's blood sugar taken within the past twelve months), the device proceeds with the process, prompting the subject to indicate whether the subject has knowledge of a first blood sugar level (e.g., an HbA1c blood sugar level of the subject) and applies (808) the answer received from the subject to a first blood sugar filter.

When the first blood sugar filter is fired (e.g., when the answer to the prompt indicates the subject knows their HbA1c level), the device proceeds with the qualification process, prompting the subject to provide information about their first blood sugar level and applies (810) the answer received from the subject to a second blood sugar filter. When the second blood sugar filter is not fired (e.g., when the answer indicates the subject has a first blood sugar level that satisfied a threshold blood sugar level criteria), the process continues, prompting the subject to providing information about their gender.

When the first blood sugar filter is not fired, the device proceeds with the qualification process, prompting the subject to provide information about their second blood sugar level (e.g., fasting glucose blood sugar level) and then applies (812) the answer received from the subject to a third blood sugar filter. When the third blood sugar filter is not fired (e.g., when the answer indicates the subject has a second blood sugar level that is above a second ceiling blood sugar level), the process terminates (805) without authorizing provision of the gliflozin Sodium-Glucose Cotransport 2 inhibitor pharmaceutical agent and, optionally, transmits advice to the user to return later when their blood sugar level is within the floor and ceiling levels.

When the second blood sugar filter or the third blood sugar filter is fired, the device proceeds with the qualification process, prompting the subject to provide information about their gender and then applies (814) the answer received from the subject to a gender filter. When the gender filter is fired (e.g., when the answer indicates the subject was identified as male at birth), the device proceeds with the qualification process, prompting the subject to provide information about a diabetes medication status of the subject.

When the gender filter is not fired (e.g., when the answer indicates the subject was identified as female at birth), the device proceeds with the qualification process, prompting the subject to provide information about their pregnancy status and then applies (816) the answer received from the subject to a pregnancy filter. When the pregnancy filter is fired (e.g., when the answer indicates the subject is pregnant, breastfeeding, or planning to become pregnant), the device terminates (801) the qualification process without authorizing provision of the gliflozin Sodium-Glucose Cotransport 2 inhibitor pharmaceutical agent and, optionally, transmits advice to the user as to why they should not take the gliflozin Sodium-Glucose Cotransport 2 inhibitor pharmaceutical agent.

When the pregnancy filter is not fired, the device proceeds with the qualification process, prompting the subject to provide information indicating whether they are taking a diabetes medication and applies (818) the answer to a diabetes medication filter. When the diabetes symptom filter is fired (e.g., when the subject's answer indicates the subject has started taking a diabetes medication since receiving their last provision of the gliflozin Sodium-Glucose Cotransport 2 inhibitor pharmaceutical composition), the device terminates (805) the qualification process, optionally transmitting advice for the subject to discuss taking a gliflozin Sodium-Glucose Cotransport 2 inhibitor pharmaceutical agent with a medical professional.

When the diabetes medication filter is not fired, the device proceeds with the qualification process, prompting the subject to provide information indicating whether they have developed a kidney disease and applies (820) the answer to a kidney disease filter. When the kidney disease filter is fired (e.g., when the subject's answer indicates the subject has developed a kidney disease since receiving their last provision of the gliflozin Sodium-Glucose Cotransport 2 inhibit pharmaceutical composition), the device terminates (805) the qualification process, optionally transmitting advice for the subject to discuss taking a gliflozin Sodium-Glucose Cotransport 2 inhibitor pharmaceutical agent with a medical professional.

When the kidney disease filter is not fired, the device proceeds with the qualification process, prompting the subject to provide information indicating whether they have developed a kidney problem and applies (822) the answer to a kidney problem filter. When the kidney problem filter is fired (e.g., when the subject's answer indicates the subject has developed a kidney problem symptom since receiving their last provision of the gliflozin Sodium-Glucose Cotransport 2 inhibitor pharmaceutical composition), the device creates (821-1) a record of an adverse event (e.g., aggregated in an adverse event data store having records of adverse events from a plurality of users) and the device terminates (805) the qualification process, optionally transmitting advice for the subject to discuss taking a gliflozin Sodium-Glucose Cotransport 2 inhibitor pharmaceutical agent with a medical professional.

When the kidney problem filter is not fired, the device proceeds with the qualification process, prompting the subject to provide information indicating whether they have developed a urinary problem and applies (824) the answer to a urinary problem filter. When the urinary problem filter is fired (e.g., when the subject's answer indicates the subject has developed a symptom of a urinary problem since receiving their last provision of the gliflozin Sodium-Glucose Cotransport 2 inhibitor pharmaceutical composition), the device creates (821-2) a record of an adverse event (e.g., aggregated in an adverse event data store having records of adverse events from a plurality of users) and the device terminates (805) the qualification process, optionally transmitting advice for the subject to discuss taking a gliflozin Sodium-Glucose Cotransport 2 inhibitor pharmaceutical agent with a medical professional.

When the urinary problem filter is not fired, the device proceeds with the qualification process, prompting the subject to provide information indicating whether they have experienced a yeast infection and then applies (826) the answer received from the subject to a yeast infection filter. When the yeast infection filter is fired (e.g., when the answer indicates the subject has experienced a yeast infection since receiving their last provision of the gliflozin Sodium-Glucose Cotransport 2 inhibitor pharmaceutical composition), the device creates (821-3) a record of an adverse event (e.g., aggregated in an adverse event data store having records of adverse events from a plurality of users) and the device terminates (803) the qualification process, optionally transmitting advice for the subject to discuss taking a gliflozin Sodium-Glucose Cotransport 2 inhibitor pharmaceutical agent with a medical professional

When the yeast infection filter is not fired, the device proceeds with the re-qualification process, prompting (828) the subject to confirm their answers. If the user confirms their answers, the device transmits (830) a drug facts label for the gliflozin Sodium-Glucose Cotransport 2 inhibitor pharmaceutical composition and prompts the user to read the drug facts label. If the subject confirms they have read the drug facts label, the device proceeds to authorize (832) purchase of the gliflozin Sodium-Glucose Cotransport 2 inhibitor pharmaceutical composition.

### Examples

Example 1—Canagliflozin: A computer system is configured for qualifying a subject for over-the-counter delivery of a canagliflozin pharmaceutical composition to treat or prevent prediabetes (e.g., by lowering blood glucose). The computer system includes instructions for conducting an assessment survey of the subject. The assessment survey is used to obtain one or more of the following results of: the subject's age, whether the subject is one of pregnant, breastfeeding, or planning to become pregnant, whether the subject is diagnosed as diabetic, whether the subjects taking a medication used to treat diabetes, whether the subject has kidney disease, whether the subject has elevated fasting blood glucose (or other similar indication of prediabetes, e.g., elevated HbA1c), and whether the subject's BMI is greater than a specified threshold value (alternatively, subject is prompted to input height and weight for a BMI calculation).

The computer system is configured to run assessment survey results against a first series of assessment filters that are each associated with a first filter category class. The filters of the first filter category class are configured to prevent authorization for OTC delivery of the canagliflozin pharmaceutical composition when the assessment survey results indicate the presence of a contraindication for administration of over-the-counter canagliflozin. The first series of assessment filters includes an age assessment filter, a pregnancy assessment filter, a diabetes assessment filter, a diabetes medication assessment filter, a kidney disease assessment filter, a blood glucose assessment filter, an allergy assessment filter, and a BMI assessment filter, configured to be fired as described above.

The computer system is configured to run assessment survey results against a second series of assessment filters that are each associated with a second filter category class. The filters of the second category filter class are configured to generate a warning when the assessment survey results indicate the presence of a risk factor for administration of over-the-counter canagliflozin. The second series of assessment filters includes a urinary tract infection assessment filter, a pregnancy assessment filter, a hypotension assessment filter, a bone fracture assessment filter, a moderate drug interaction assessment filter, and an amputation risk assessment filter, configured to be fired as described above. The computer is configured to prompt the subject to acknowledge having discussed any warnings that are triggered with a medical professional (e.g., their physician or healthcare provider).

The computer system is configured to proceed with a fulfillment process only when none of the first series of assessment filters are fired and the subject has acknowledged each warning associated with second series of assessment filters that are fired. The computer system stores an indication of an initial order of the OTC canagliflozin in a subject profile, and communicates an over-the-counter drug facts label for the canagliflozin pharmaceutical composition to the subject. Upon confirmation by the subject that they have received and read the over-the-counter drug facts label, the computer system is configured to authorize provision of the OTC canagliflozin pharmaceutical composition to the subject.

In some embodiments, the computer system includes instructions for qualifying a subject for a re-order provision of the canagliflozin pharmaceutical composition. The computer system is configured to conduct a reassessment survey responsive to a re-order request for the canagliflozin pharmaceutical composition by the subject. The reassessment survey is utilized to obtain one or more results of whether the subject is one of pregnant, breastfeeding, or planning to become pregnant, whether the subject is taking a medication used to treat diabetes, whether the subject has developed kidney disease since receiving their last provision of the canagliflozin pharmaceutical composition, whether the subject has experienced symptoms of a urinary tract infection since receiving their last provision of the canagliflozin pharmaceutical composition, whether the subject has experienced symptoms of a bacterial infection since receiving their last provision of the canagliflozin pharmaceutical composition, whether the subject's low-density lipoprotein cholesterol (LDL-C) level has increased since receiving their last provision of the canagliflozin pharmaceutical composition, whether the subject has elevated fasting blood glucose (or other similar indication of prediabetes, e.g., elevated HbA1c) since receiving their last provision of the canagliflozin pharmaceutical composition, and whether the subject's BMI remains greater than a threshold value since receiving their last provision of the canagliflozin pharmaceutical composition.

The computer system is configured to run reassessment survey results against a first series of reassessment filters that are each associated with the first filter category class. In some embodiments, the first series of reassessment filters includes a pregnancy reassessment filter, a diabetes medication reassessment filter, a kidney disease reassessment filter, a urinary tract infection reassessment filter, a bacterial infection reassessment filter, a cholesterol (LDL-C) reassessment filter, a blood glucose reassessment filter, and a BMI reassessment filter, configured to be fired as described above.

The computer system then proceeds with a re-fulfillment process only when none of the first series of reassessment filters are fired. The computer system stores an indication of a re-order of the OTC canagliflozin in the subject profile, and communicates an over-the-counter drug facts label for the canagliflozin pharmaceutical composition to the subject. Upon confirmation by the subject that they have received and read the over-the-counter drug facts label, the computer system is configured to authorize provision of the OTC canagliflozin pharmaceutical composition to the subject.

Example 2—Ertugliflozin: A computer system is configured for qualifying a subject for over-the-counter delivery of a ertugliflozin pharmaceutical composition to treat or prevent prediabetes (e.g., by lowering blood glucose). The computer system includes instructions for conducting an assessment survey of the subject. The assessment survey is used to obtain one or more of the following results of: the subject's age, whether the subject is one of pregnant, breastfeeding, or planning to become pregnant, whether the subject is diagnosed as diabetic, whether the subjects taking a medication used to treat diabetes, whether the subject has kidney disease, whether the subject has elevated fasting blood glucose (or other similar indication of prediabetes, e.g., elevated HbA1c), and whether the subject's BMI is greater than a specified threshold value (alternatively, subject is prompted to input height and weight for a BMI calculation).

The computer system is configured to run assessment survey results against a first series of assessment filters that are each associated with a first filter category class. The filters of the first filter category class are configured to prevent authorization for OTC delivery of the ertugliflozin pharmaceutical composition when the assessment survey results indicate the presence of a contraindication for administration of over-the-counter ertugliflozin. The first series of assessment filters includes an age assessment filter, a pregnancy assessment filter, a diabetes assessment filter, a diabetes medication assessment filter, a kidney disease assessment filter, a blood glucose assessment filter, an allergy assessment filter, and a BMI assessment filter, configured to be fired as described above.

The computer system is configured to run assessment survey results against a second series of assessment filters that are each associated with a second filter category class. The filters of the second category filter class are configured to generate a warning when the assessment survey results indicate the presence of a risk factor for administration of over-the-counter ertugliflozin. The second series of assessment filters includes a urinary tract infection assessment filter, a pregnancy assessment filter, a moderate drug interaction assessment filter, and an amputation risk assessment filter, configured to be fired as described above. The computer is configured to prompt the subject to acknowledge having discussed any warnings that are triggered with a medical professional (e.g., their physician or healthcare provider).

The computer system is configured to proceed with a fulfillment process only when none of the first series of assessment filters are fired and the subject has acknowledged each warning associated with second series of assessment filters that are fired. The computer system stores an indication of an initial order of the OTC ertugliflozin in a subject profile, and communicates an over-the-counter drug facts label for the ertugliflozin pharmaceutical composition to the subject. Upon confirmation by the subject that they have received and read the over-the-counter drug facts label, the computer system is configured to authorize provision of the OTC ertugliflozin pharmaceutical composition to the subject.

In some embodiments, the computer system includes instructions for qualifying a subject for a re-order provision of the ertugliflozin pharmaceutical composition. The computer system is configured to conduct a reassessment survey responsive to a re-order request for the ertugliflozin pharmaceutical composition by the subject. The reassessment survey is utilized to obtain one or more results of: whether the subject is one of pregnant, breastfeeding, or planning to become pregnant, whether the subject is taking a medication used to treat diabetes, whether the subject has developed kidney disease since receiving their last provision of the ertugliflozin pharmaceutical composition, whether the subject has experienced symptoms of a urinary tract infection since receiving their last provision of the ertugliflozin pharmaceutical composition, whether the subject has experienced symptoms of a bacterial infection since receiving their last provision of the ertugliflozin pharmaceutical composition, whether the subject has elevated fasting blood glucose (or other similar indication of prediabetes, e.g., elevated HbA1c) since receiving their last provision of the ertugliflozin pharmaceutical composition, and whether the subject's BMI remains greater than a threshold value since receiving their last provision of the ertugliflozin pharmaceutical composition.

The computer system is configured to run reassessment survey results against a first series of reassessment filters that are each associated with the first filter category class. In some embodiments, the first series of reassessment filters includes a pregnancy reassessment filter, a diabetes medication reassessment filter, a kidney disease reassessment filter, a urinary tract infection reassessment filter, a bacterial infection reassessment filter, a blood glucose reassessment filter, and a BMI reassessment filter, configured to be fired as described above.

The computer system then proceeds with a re-fulfillment process only when none of the first series of reassessment filters are fired. The computer system stores an indication of a re-order of the OTC ertugliflozin in the subject profile, and communicates an over-the-counter drug facts label for the ertugliflozin pharmaceutical composition to the subject. Upon confirmation by the subject that they have received and read the over-the-counter drug facts label, the computer system is configured to authorize provision of the OTC ertugliflozin pharmaceutical composition to the subject.

Example 3—Empagliflozin: A computer system is configured for qualifying a subject for over-the-counter delivery of a empagliflozin pharmaceutical composition to treat or prevent prediabetes (e.g., by lowering blood glucose). The computer system includes instructions for conducting an assessment survey of the subject. The assessment survey is used to obtain one or more of the following results of: the subject's age, whether the subject is one of pregnant, breastfeeding, or planning to become pregnant, whether the subject is diagnosed as diabetic, whether the subjects taking a medication used to treat diabetes, whether the subject has kidney disease, whether the subject has elevated fasting blood glucose (or other similar indication of prediabetes, e.g., elevated HbA1c), and whether the subject's BMI is greater than a specified threshold value (alternatively, subject is prompted to input height and weight for a BMI calculation).

The computer system is configured to run assessment survey results against a first series of assessment filters that are each associated with a first filter category class. The filters of the first filter category class are configured to prevent authorization for OTC delivery of the empagliflozin pharmaceutical composition when the assessment survey results indicate the presence of a contraindication for administration of over-the-counter empagliflozin. The first series of assessment filters includes an age assessment filter, a pregnancy assessment filter, a diabetes assessment filter, a diabetes medication assessment filter, a kidney disease assessment filter, a blood glucose assessment filter, an allergy assessment filter, and a BMI assessment filter, configured to be fired as described above.

The computer system is configured to run assessment survey results against a second series of assessment filters that are each associated with a second filter category class. The filters of the second category filter class are configured to generate a warning when the assessment survey results indicate the presence of a risk factor for administration of over-the-counter empagliflozin. The second series of assessment filters includes a urinary tract infection assessment filter, a pregnancy assessment filter, a hypotension assessment filter, and a moderate drug interaction assessment filter, configured to be fired as described above. The computer is configured to prompt the subject to acknowledge having discussed any warnings that are triggered with a medical professional (e.g., their physician or healthcare provider).

The computer system is configured to proceed with a fulfillment process only when none of the first series of assessment filters are fired and the subject has acknowledged each warning associated with second series of assessment filters that are fired. The computer system stores an indication of an initial order of the OTC empagliflozin in a subject profile, and communicates an over-the-counter drug facts label for the empagliflozin pharmaceutical composition to the subject. Upon confirmation by the subject that they have received and read the over-the-counter drug facts label, the computer system is configured to authorize provision of the OTC empagliflozin pharmaceutical composition to the subject.

In some embodiments, the computer system includes instructions for qualifying a subject for a re-order provision of the empagliflozin pharmaceutical composition. The computer system is configured to conduct a reassessment survey responsive to a re-order request for the empagliflozin pharmaceutical composition by the subject. The reassessment survey is utilized to obtain one or more results of: whether the subject is one of pregnant, breastfeeding, or planning to become pregnant, whether the subject is taking a medication used to treat diabetes, whether the subject has developed kidney disease since receiving their last provision of the empagliflozin pharmaceutical composition, whether the subject has experienced symptoms of a urinary tract infection since receiving their last provision of the empagliflozin pharmaceutical composition, whether the subject has experienced symptoms of a bacterial infection since receiving their last provision of the empagliflozin pharmaceutical composition, whether the subject's low-density lipoprotein cholesterol (LDL-C) level has increased since receiving their last provision of the empagliflozin pharmaceutical composition, whether the subject has elevated fasting blood glucose (or other similar indication of prediabetes, e.g., elevated HbA1c) since receiving their last provision of the empagliflozin pharmaceutical composition, and whether the subject's BMI remains greater than a threshold value since receiving their last provision of the empagliflozin pharmaceutical composition.

The computer system is configured to run reassessment survey results against a first series of reassessment filters that are each associated with the first filter category class. In some embodiments, the first series of reassessment filters includes a pregnancy reassessment filter, a diabetes medication reassessment filter, a kidney disease reassessment filter, a urinary tract infection reassessment filter, a bacterial infection reassessment filter, a cholesterol (LDL-C) reassessment filter, a blood glucose reassessment filter, and a BMI reassessment filter, configured to be fired as described above.

The computer system then proceeds with a re-fulfillment process only when none of the first series of reassessment filters are fired. The computer system stores an indication of a re-order of the OTC empagliflozin in the subject profile, and communicates an over-the-counter drug facts label for the empagliflozin pharmaceutical composition to the subject. Upon confirmation by the subject that they have received and read the over-the-counter drug facts label, the computer system is configured to authorize provision of the OTC empagliflozin pharmaceutical composition to the subject.

Example 4—Dapagliflozin: A computer system is configured for qualifying a subject for over-the-counter delivery of a dapagliflozin pharmaceutical composition to treat or prevent prediabetes (e.g., by lowering blood glucose). The computer system includes instructions for conducting an assessment survey of the subject. The assessment survey is used to obtain one or more of the following results of: the subject's age, whether the subject is one of pregnant, breastfeeding, or planning to become pregnant, whether the subject is diagnosed as diabetic, whether the subjects taking a medication used to treat diabetes, whether the subject has kidney disease, whether the subject has elevated fasting blood glucose (or other similar indication of prediabetes, e.g., elevated HbA1c), and whether the subject's BMI is greater than a specified threshold value (alternatively, subject is prompted to input height and weight for a BMI calculation).

The computer system is configured to run assessment survey results against a first series of assessment filters that are each associated with a first filter category class. The filters of the first filter category class are configured to prevent authorization for OTC delivery of the dapagliflozin pharmaceutical composition when the assessment survey results indicate the presence of a contraindication for administration of over-the-counter dapagliflozin. The first series of assessment filters includes an age assessment filter, a pregnancy assessment filter, a diabetes assessment filter, a diabetes medication assessment filter, a kidney disease assessment filter, a blood glucose assessment filter, an allergy assessment filter, and a BMI assessment filter, configured to be fired as described above.

The computer system is configured to run assessment survey results against a second series of assessment filters that are each associated with a second filter category class. The filters of the second category filter class are configured to generate a warning when the assessment survey results indicate the presence of a risk factor for administration of over-the-counter dapagliflozin. The second series of assessment filters includes a urinary tract infection assessment filter, a pregnancy assessment filter, a hypotension assessment filter, and a moderate drug interaction assessment filter, configured to be fired as described above. The computer is configured to prompt the subject to acknowledge having discussed any warnings that are triggered with a medical professional (e.g., their physician or healthcare provider).

The computer system is configured to proceed with a fulfillment process only when none of the first series of assessment filters are fired and the subject has acknowledged each warning associated with second series of assessment filters that are fired. The computer system stores an indication of an initial order of the OTC dapagliflozin in a subject profile, and communicates an over-the-counter drug facts label for the dapagliflozin pharmaceutical composition to the subject. Upon confirmation by the subject that they have received and read the over-the-counter drug facts label, the computer system is configured to authorize provision of the OTC dapagliflozin pharmaceutical composition to the subject.

In some embodiments, the computer system includes instructions for qualifying a subject for a re-order provision of the dapagliflozin pharmaceutical composition. The computer system is configured to conduct a reassessment survey responsive to a re-order request for the dapagliflozin pharmaceutical composition by the subject. The reassessment survey is utilized to obtain one or more results of: whether the subject is one of pregnant, breastfeeding, or planning to become pregnant, whether the subject is taking a medication used to treat diabetes, whether the subject has developed kidney disease since receiving their last provision of the dapagliflozin pharmaceutical composition, whether the subject has experienced symptoms of a urinary tract infection since receiving their last provision of the dapagliflozin pharmaceutical composition, whether the subject has experienced symptoms of a bacterial infection since receiving their last provision of the dapagliflozin pharmaceutical composition, whether the subject has elevated fasting blood glucose (or other similar indication of prediabetes, e.g., elevated HbA1c) since receiving their last provision of the dapagliflozin pharmaceutical composition, and whether the subject's BMI remains greater than a threshold value since receiving their last provision of the dapagliflozin pharmaceutical composition.

The computer system is configured to run reassessment survey results against a first series of reassessment filters that are each associated with the first filter category class. In some embodiments, the first series of reassessment filters includes a pregnancy reassessment filter, a diabetes medication reassessment filter, a kidney disease reassessment filter, a urinary tract infection reassessment filter, a bacterial infection reassessment filter, a blood glucose reassessment filter, and a BMI reassessment filter, configured to be fired as described above.

The computer system then proceeds with a re-fulfillment process only when none of the first series of reassessment filters are fired. The computer system stores an indication of a re-order of the OTC dapagliflozin in the subject profile, and communicates an over-the-counter drug facts label for the dapagliflozin pharmaceutical composition to the subject. Upon confirmation by the subject that they have received and read the over-the-counter drug facts label, the computer system is configured to authorize provision of the OTC dapagliflozin pharmaceutical composition to the subject.

Example 5—Dapagliflozin propanediol: A computer system is configured for qualifying a subject for over-the-counter delivery of a dapagliflozin propanediol pharmaceutical composition to treat or prevent prediabetes (e.g., by lowering blood glucose). The computer system includes instructions for conducting an assessment survey of the subject. The assessment survey is used to obtain one or more of the following results of the subject's age, whether the subject is one of pregnant, breastfeeding, or planning to become pregnant, whether the subject is diagnosed as diabetic, whether the subjects taking a medication used to treat diabetes, whether the subject has kidney disease, whether the subject has elevated fasting blood glucose (or other similar indication of prediabetes, e.g., elevated HbA1c), and whether the subject's BMI is greater than a specified threshold value (alternatively, subject is prompted to input height and weight for a BMI calculation).

The computer system is configured to run assessment survey results against a first series of assessment filters that are each associated with a first filter category class. The filters of the first filter category class are configured to prevent authorization for OTC delivery of the dapagliflozin propanediol pharmaceutical composition when the assessment survey results indicate the presence of a contraindication for administration of over-the-counter dapagliflozin propanediol. The first series of assessment filters includes an age assessment filter, a pregnancy assessment filter, a diabetes assessment filter, a diabetes medication assessment filter, a kidney disease assessment filter, a blood glucose assessment filter, an allergy assessment filter, and a BMI assessment filter, configured to be fired as described above.

The computer system is configured to run assessment survey results against a second series of assessment filters that are each associated with a second filter category class. The filters of the second category filter class are configured to generate a warning when the assessment survey results indicate the presence of a risk factor for administration of over-the-counter dapagliflozin propanediol. The second series of assessment filters includes a heart failure assessment filter, a urinary tract infection assessment filter, a pregnancy assessment filter, a hypotension assessment filter, and a moderate drug interaction assessment filter, configured to be fired as described above. The computer is configured to prompt the subject to acknowledge having discussed any warnings that are triggered with a medical professional (e.g., their physician or healthcare provider).

The computer system is configured to proceed with a fulfillment process only when none of the first series of assessment filters are fired and the subject has acknowledged each warning associated with second series of assessment filters that are fired. The computer system stores an indication of an initial order of the OTC dapagliflozin propanediol in a subject profile, and communicates an over-the-counter drug facts label for the dapagliflozin propanediol pharmaceutical composition to the subject. Upon confirmation by the subject that they have received and read the over-the-counter drug facts label, the computer system is configured to authorize provision of the OTC dapagliflozin propanediol pharmaceutical composition to the subject.

In some embodiments, the computer system includes instructions for qualifying a subject for a re-order provision of the dapagliflozin propanediol pharmaceutical composition. The computer system is configured to conduct a reassessment survey responsive to a re-order request for the dapagliflozin propanediol pharmaceutical composition by the subject. The reassessment survey is utilized to obtain one or more results of: whether the subject is one of pregnant, breastfeeding, or planning to become pregnant, whether the subject is taking a medication used to treat diabetes, whether the subject has developed kidney disease since receiving their last provision of the dapagliflozin propanediol pharmaceutical composition, whether the subject has experienced symptoms of a urinary tract infection since receiving their last provision of the dapagliflozin propanediol pharmaceutical composition, whether the subject has experienced symptoms of a bacterial infection since receiving their last provision of the dapagliflozin propanediol pharmaceutical composition, whether the subject has experienced symptoms of pancreatitis since receiving their last provision of the dapagliflozin propanediol pharmaceutical composition, whether the subject has experienced arthralgia since receiving their last provision of the dapagliflozin propanediol pharmaceutical composition, whether the subject has developed blisters or erosions since receiving their last provision of the dapagliflozin propanediol pharmaceutical composition, whether the subject has elevated fasting blood glucose (or other similar indication of prediabetes, e.g., elevated HbA1c) since receiving their last provision of the dapagliflozin propanediol pharmaceutical composition, and whether the subject's BMI remains greater than a threshold value since receiving their last provision of the dapagliflozin propanediol pharmaceutical composition.

The computer system is configured to run reassessment survey results against a first series of reassessment filters that are each associated with the first filter category class. In some embodiments, the first series of reassessment filters includes a pregnancy reassessment filter, a diabetes medication reassessment filter, a kidney disease reassessment filter, a urinary tract infection reassessment filter, a bacterial infection reassessment filter, a pancreatitis reassessment filter, an arthralgia reassessment filter, a bulbous pemphigoid reassessment filter, a blood glucose reassessment filter, and a BMI reassessment filter, configured to be fired as described above.

The computer system then proceeds with a re-fulfillment process only when none of the first series of reassessment filters are fired. The computer system stores an indication of a re-order of the OTC dapagliflozin propanediol in the subject profile, and communicates an over-the-counter drug facts label for the dapagliflozin propanediol pharmaceutical composition to the subject. Upon confirmation by the subject that they have received and read the over-the-counter drug facts label, the computer system is configured to authorize provision of the OTC dapagliflozin propanediol pharmaceutical composition to the subject.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that includes a computer program mechanism embedded in a non-transitory computer readable storage medium. For instance, the computer program product could contain the program modules shown in any combination of FIGS. 1, 2, and 3 and/or described in FIG. 4 or 5. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed is:

1. A method for authorizing an over-the-counter provision of a gliflozin Sodium-Glucose Cotransport 2 inhibitor pharmaceutical composition to a human subject, the method comprising:
A) obtaining an assessment information set about the subject, via a first computer system having a processor programed to obtain the assessment information set, the assessment information set comprising:
an age of the subject,
a gender of the subject,
when the subject is a female, whether the subject is pregnant or breastfeeding,
whether the subject has type I or type II diabetes,
whether the subject is taking a diabetes medication,
an indication of whether the subject has kidney disease,
an indication of the subject's blood glucose level, and
an indication of whether the subject has a history of urinary tract infections;
B) applying an algorithm to the assessment information set, via a second computer system having a processor programed to perform the algorithm, wherein the algorithm:
i) runs all or a portion of the assessment information set against a first set of assessment filters, wherein the subject is deemed not qualified for the over-the-counter provision of the gliflozin Sodium-Glucose Cotransport 2 inhibitor pharmaceutical composition when a filter in the first set of assessment filters is fired and the method is terminated without authorizing the over-the-counter provision of the gliflozin Sodium-Glucose Cotransport 2 inhibitor pharmaceutical composition to the subject, wherein the first set of assessment filters comprises:
an age assessment filter that is fired at least when the assessment information set indicates the age of the subject fails to satisfy an age threshold for receiving the gliflozin Sodium-Glucose Cotransport 2 inhibitor pharmaceutical composition,
when the subject is a female, a pregnancy assessment filter that is fired at least when the assessment information set indicates the subject is pregnant or breastfeeding,
a diabetes assessment filter that is fired at least when the assessment information set indicates the subject has type 1 or type 2 diabetes,
a diabetes medication assessment filter that is fired at least when the assessment information set indicates the subject is taking a diabetes medication,
a kidney disease assessment filter that is fired at least when the assessment information set indicates that the subject has kidney disease, and
a blood glucose assessment filter that is fired at least when the assessment information set indicates that the subject does not have prediabetes,
ii) runs all or a portion of the assessment information set against a second set of assessment filters, wherein, when a respective filter in the second set of assessment filters is fired, the subject is provided with a warning corresponding to the respective filter, and wherein the second set of assessment filters comprises:
a urinary tract infection assessment filter that is fired at least when the assessment information set indicates that the subject has a history of urinary tract infections; and
iii) obtains, when a respective filter in the second set of assessment filters is fired, acknowledgment from the subject confirming that the subject has acknowledged the risk factor associated with each warning issued to the subject by any filter in the second set of assessment filters; and
iv) proceeds with a fulfillment process when (a) no filter in the first set of assessment filters has been fired and (b), when a respective filter in the second set of assessment filters is fired, the subject has acknowledged each warning associated with each filter in the second set of assessment filters that was fired, wherein the fulfillment process comprises:
storing an indication in a subject profile of an initial authorization for the over-the-counter provision of the gliflozin Sodium-Glucose Cotransport 2 inhibitor pharmaceutical composition using the processor of the second computer system, and
authorizing the over-the-counter provision of the gliflozin Sodium-Glucose Cotransport 2 inhibitor pharmaceutical composition to the subject.

2. A method for authorizing a human subject for an over-the-counter provision of a gliflozin Sodium-Glucose Cotransport 2 inhibitor pharmaceutical composition, the method comprising:
A) obtaining an assessment information set about the subject, via a first computer system having a processor programed to obtain the assessment information set, the assessment information set comprising:
an age of the subject,
a gender of the subject,
when the subject is a female, whether the subject is pregnant or breastfeeding,
whether the subject has type I or type II diabetes,
whether the subject is taking a diabetes medication,
an indication of whether the subject has kidney disease,
an indication of the subject's blood glucose level, and
an indication of whether the subject has a history of urinary tract infections;
B) applying an algorithm to the assessment information set, via a second computer system having a processor programed to perform the algorithm, wherein the algorithm:
i) runs all or a portion of the assessment information set against a plurality of filters, wherein, when a respective filter in the plurality of filters is fired (1) the method is terminated without authorizing the over-the-counter provision of the gliflozin Sodium-Glucose Cotransport 2 inhibitor pharmaceutical composition to the subject or (2) the subject is provided with a warning corresponding to the respective filter, and wherein the plurality of filters comprises:
an age assessment filter that is fired at least when the assessment information set indicates the age of the subject fails to satisfy an age threshold for receiving the gliflozin Sodium-Glucose Cotransport 2 inhibitor pharmaceutical composition,
when the subject is a female, a pregnancy assessment filter that is fired at least when the assessment information set indicates the subject is pregnant or breastfeeding,
a diabetes assessment filter that is fired at least when the assessment information set indicates the subject has type 1 or type 2 diabetes,
a diabetes medication assessment filter that is fired at least when the assessment information set indicates the subject is taking a diabetes medication,
a kidney disease assessment filter that is fired at least when the assessment information set indicates that the subject has kidney disease,
a blood glucose assessment filter that is fired at least when the assessment information set indicates that the subject does not have prediabetes, and
a urinary tract infection assessment filter that is fired at least when the assessment information set indicates that the subject has a history of urinary tract infections;
ii) obtains, when (1) the method is not already terminated by the firing of a filter in the plurality of filters and (2) a respective filter in the plurality of filters has fired, acknowledgment from the subject for each warning issued to the subject by any filter in the plurality of filters, and
iii) proceeds with a fulfillment process when (1) the method is not already terminated by the firing of a filter in the plurality of filters and (2) the subject has acknowledged each warning associated with each filter in the plurality of filters that was fired and that is associated with a warning, wherein the fulfillment process comprises:
storing an indication in a subject profile of an initial authorization for the over-the-counter provision of the gliflozin Sodium-Glucose Cotransport 2 inhibitor pharmaceutical composition using the processor of the second computer system, and
authorizing the provision of the gliflozin Sodium-Glucose Cotransport 2 inhibitor pharmaceutical composition to the subject.

3. The method of claim 2, wherein the plurality of filters comprises at least one filter that terminates the method when fired.

4. The method of claim 2 or 3, wherein the plurality of filters comprises at least one respective filter that provides a warning corresponding to the respective filter when fired.

5. The method of claim 2 or 3, wherein the plurality of filters comprises at least two, three, four, or five respective filters that each provides a warning corresponding to the respective filter when fired.

6. The method of claim 2 or 3, wherein the plurality of filters comprises less than five, four, three, or two respective filters that each provides a warning corresponding to the respective filter when fired.

7. The method of any one of claims 1-6, wherein the obtaining A) comprises providing an assessment survey to the subject.

8. The method of any one of claims 1-3, wherein the age assessment filter is fired when the assessment information set indicates the subject is less than 18 years of age.

9. The method of any one of claims 1-8, wherein the pregnancy assessment filter is also fired when the assessment information set indicates the subject is female and planning on becoming pregnant.

10. The method of any one of claims 1-9, wherein:
the indication of whether the subject has kidney disease in the assessment information set includes whether the subject has been diagnosed with kidney disease, and
the kidney disease assessment filter is fired at least when the assessment information set indicates the subject has been diagnosed with kidney disease.

11. The method of any one of claims 1-10, wherein:
the indication of whether the subject has kidney disease in the assessment information set includes whether the subject is receiving kidney dialysis, and
the kidney disease assessment filter is fired at least when the assessment information set indicates the subject is receiving kidney dialysis.

12. The method of any one of claims 1-11, wherein the blood glucose assessment filter is fired when the assessment information set indicates:
the subject has a hemoglobin A1C (HbA1c) level of less than 5.7%, or
the subject has an HbA1c level of greater than 6.4%.

13. The method of any one of claims 1-12, wherein the blood glucose assessment filter is fired when the assessment information set indicates:
the subject has a fasting blood glucose level of less than 100 mg/dL, or
the subject has a fasting blood glucose level of greater than 125 mg/dL.

14. The method of any one of claims 1-13, wherein the blood glucose assessment filter is fired when the assessment information set indicates:
the subject had a blood glucose level of no more than 140 mg/dL two hours after consuming 75 grams of glucose, or
the subject has a blood glucose level of at least 200 mg/dL two hours after consuming 75 grams of glucose.

15. The method of any one of claims 1-14, wherein the urinary tract infection assessment filter is fired at least when the assessment information set indicates the subject has had problems urinating.

16. The method of any one of claims 1-15, wherein the indication of the initial authorization stored in the subject profile is an indication of an initial order for the over-the-counter provision of the gliflozin Sodium-Glucose Cotransport 2 inhibitor pharmaceutical composition.

17. The method of any one of claims 1-16, wherein:
the fulfillment process further comprises communicating an over-the-counter drug facts label for the gliflozin Sodium-Glucose Cotransport 2 inhibitor pharmaceutical composition to the subject, and
the over-the-counter provision of the gliflozin Sodium-Glucose Cotransport 2 inhibitor pharmaceutical composition is authorized upon condition of receipt of confirmation from the subject that the over-the-counter drug facts label has been received and read.

18. The method of any one of claims 1-17, wherein the fulfillment process further comprises storing a destination associated with the subject in the subject profile.

19. The method of any one of claims 1-18, wherein the fulfillment process further comprises coordinating shipping of the gliflozin Sodium-Glucose Cotransport 2 inhibitor pharmaceutical composition to a physical address associated with the subject.

20. The method of any one of claims 1-19, further comprising administering, upon authorization of the provision, the gliflozin Sodium-Glucose Cotransport 2 inhibitor pharmaceutical composition to the subject.

21. The method of any one of claims 1-20, wherein the first computer system and second computer system are the same computer system.

22. The method of any one of claims 1 and 3-21, wherein:
the assessment information set includes an indication that the subject has an elevated body mass index (BMI), and
the first set of assessment filters includes a BMI assessment filter that is fired at least when the assessment information set includes an indication that the subject's BMI is greater than or equal to 25.

23. The method of any one of claims 2-21, wherein:
the assessment information set includes an indication of whether the subject has an elevated BMI, and
the plurality of filters includes a BMI filter that is fired at least when the assessment information set includes an indication that the subject's BMI is greater than or equal to 25.

24. The method of any one of claims 1 and 3-23, wherein:
the gliflozin Sodium-Glucose Cotransport 2 inhibitor pharmaceutical composition comprises canagliflozin as an active ingredient,
the assessment information set includes an indication of whether the subject has an increased risk for bone fractures, and
the second set of assessment filters includes a bone fracture assessment filter that is fired at least when the assessment information set includes an indication that the subject has an increased risk for bone fractures.

25. The method of any one of claims 2-23, wherein:
the gliflozin Sodium-Glucose Cotransport 2 inhibitor pharmaceutical composition comprises canagliflozin as an active ingredient,
the assessment information set includes an indication of whether the subject has an increased risk for bone fractures, and
the plurality of filters includes a bone fracture assessment filter that is fired at least when the assessment information set includes an indication that the subject has an increased risk for bone fractures.

26. The method of any one of claims 1, 3-24, wherein:
the gliflozin Sodium-Glucose Cotransport 2 inhibitor pharmaceutical composition comprises canagliflozin as an active ingredient,
the assessment information set includes an indication of whether the subject is taking a medication that interacts with canagliflozin, and
the second set of assessment filters includes a moderate drug interaction assessment filter that is fired at least when the assessment information set includes an indication that the subject is taking a medication that interacts with canagliflozin.

27. The method of any one of claims 2-23 and 25, wherein:
the gliflozin Sodium-Glucose Cotransport 2 inhibitor pharmaceutical composition comprises canagliflozin as an active ingredient,
the assessment information set includes an indication of whether the subject is taking a medication that interacts with canagliflozin, and
the plurality filters includes a moderate drug interaction assessment filter that is fired at least when the assessment information set includes an indication that the subject is taking a medication that interacts with canagliflozin.

28. The method of any one of claims 1, 3-24, and 26, wherein:
the gliflozin Sodium-Glucose Cotransport 2 inhibitor pharmaceutical composition comprises canagliflozin as an active ingredient,
the assessment information set includes an indication of whether the subject has an increased risk of lower limb amputation, and
the second set of assessment filters includes an amputation risk assessment filter that is fired at least when the assessment information set includes an indication that the subject has an increased risk of lower limb amputation.

29. The method of any one of claims 2-21, 25, and 27, wherein:
the gliflozin Sodium-Glucose Cotransport 2 inhibitor pharmaceutical composition comprises canagliflozin as an active ingredient,
the assessment information set includes an indication of whether the subject has an increased risk of lower limb amputation, and
the plurality filters includes an amputation risk assessment filter that is fired at least when the assessment information set includes an indication that the subject has an increased risk of lower limb amputation.

30. The method of claim 28 or 29, wherein the indication that the subject has an increased risk of lower limb amputation is selected from the group consisting of a history of amputation, a history of heart disease, a high risk of heart disease, blocked or narrowed blood vessels, leg neuropathy, and diabetic foot ulcers or sores.

31. The method of any one of claims 1 and 3-23, wherein:
the gliflozin Sodium-Glucose Cotransport 2 inhibitor pharmaceutical composition comprises ertugliflozin as an active ingredient,
the assessment information set includes an indication of whether the subject has a history of amputation, and
the second set of assessment filters includes an amputation history assessment filter that is fired at least when the assessment information set includes an indication that the subject has a history of amputation.

32. The method of any one of claims 2-23, wherein:
the gliflozin Sodium-Glucose Cotransport 2 inhibitor pharmaceutical composition comprises ertugliflozin as an active ingredient,
the assessment information set includes an indication of whether the subject has a history of amputation, and
the plurality filters includes an amputation history assessment filter that is fired at least when the assessment information set includes an indication that the subject has a history of amputation.

33. A method for re-authorizing a human subject who was previously authorized for an over-the-counter provision of a gliflozin Sodium-Glucose Cotransport 2 inhibitor pharmaceutical composition, the method comprising:
A) receiving a re-order request from the subject for the gliflozin Sodium-Glucose Cotransport 2 inhibitor pharmaceutical composition, at a first computer system having a processor programmed to receive the re-order request;
B) obtaining a reassessment information set about the subject, via a second computer system having a processor programed to obtain the reassessment information set, the reassessment information set comprising:
when the subject is a female, whether the subject is pregnant or breastfeeding,
whether the subject has started taking a diabetes medication other than the over-the-counter provision of the gliflozin Sodium-Glucose Cotransport 2 inhibitor pharmaceutical composition since receiving their last over-the-counter provision of the gliflozin Sodium-Glucose Cotransport 2 inhibitor pharmaceutical composition,
an indication of whether the subject has developed kidney disease since receiving their last over-the-counter provision of the gliflozin Sodium-Glucose Cotransport 2 inhibitor pharmaceutical composition,
an indication of whether the subject has experienced a urinary tract infection since receiving their last over-the-counter provision of the gliflozin Sodium-Glucose Cotransport 2 inhibitor pharmaceutical composition, and
an indication of whether the subject has experienced a bacterial infection since receiving their last over-the-counter provision of the gliflozin Sodium-Glucose Cotransport 2 inhibitor pharmaceutical composition,
C) applying an algorithm to the reassessment information set, via a third computer system having a processor programed to perform the algorithm, wherein the algorithm:
i) runs all or a portion of the reassessment information set against a first set of reassessment filters, wherein the subject is deemed not qualified for the over-the-counter provision of the gliflozin Sodium-Glucose Cotransport 2 inhibitor pharmaceutical composition when a respective reassessment filter in the first set of reassessment filters is fired and the method is terminated without re-authorizing a provision of the gliflozin Sodium-Glucose Cotransport 2 inhibitor pharmaceutical composition to the subject, wherein the first set of reassessment filters comprises:
when the subject is a female, a pregnancy reassessment filter that is fired at least when the reassessment information set indicates the subject is pregnant or breastfeeding,
a diabetes medication reassessment filter that is fired at least when the reassessment information set indicates the subject has started taking a medication for treating diabetes other than the gliflozin Sodium-Glucose Cotransport 2 inhibitor pharmaceutical composition since receiving their last over-the-counter provision of the gliflozin Sodium-Glucose Cotransport 2 inhibitor pharmaceutical composition,
a kidney disease reassessment filter that is fired at least when the reassessment information set indicates that the subject has developed kidney disease since receiving their last over-the-counter provision of the gliflozin Sodium-Glucose Cotransport 2 inhibitor pharmaceutical composition,
a urinary tract infection reassessment filter that is fired at least when the reassessment information set indicates that the subject has experienced symptoms of a urinary tract infection since receiving their last over-the-counter provision of the gliflozin Sodium-Glucose Cotransport 2 inhibitor pharmaceutical composition, and
a bacterial infection reassessment filter that is fired at least when the reassessment information set indicates that the subject has experienced symptoms of a bacterial infection since receiving their last over-the-counter provision of the gliflozin Sodium-Glucose Cotransport 2 inhibitor pharmaceutical composition, and
ii) proceeds with a fulfillment process when no filter in the first set of reassessment filters has been fired, wherein the fulfillment process comprises:
storing an indication in a subject profile of a reauthorization for the gliflozin Sodium-Glucose Cotransport 2 inhibitor pharmaceutical composition using the processor of the third computer, and
authorizing the over-the-counter provision of the gliflozin Sodium-Glucose Cotransport 2 inhibitor pharmaceutical composition to the subject.

34. A method for re-authorizing a human subject who was previously authorized for an over-the-counter provision of a gliflozin Sodium-Glucose Cotransport 2 inhibitor pharmaceutical composition, the method comprising:
A) receiving a re-authorization request from the subject for the gliflozin Sodium-Glucose Cotransport 2 inhibitor pharmaceutical composition, at a first computer system having a processor programmed to receive the re-order request;
B) obtaining a reassessment information set about the subject, via a second computer system having a processor programed to obtain the reassessment information set, the reassessment information set comprising:
when the subject is a female, whether the subject is pregnant or breastfeeding,
whether the subject has started taking a medication for treating diabetes other than the gliflozin Sodium-Glucose Cotransport 2 inhibitor pharmaceutical composition since receiving their last over-the-counter provision of the gliflozin Sodium-Glucose Cotransport 2 inhibitor pharmaceutical composition,
an indication of whether the subject has developed kidney disease since receiving their last over-the-counter provision of the gliflozin Sodium-Glucose Cotransport 2 inhibitor pharmaceutical composition,
an indication of whether the subject has experienced a urinary tract infection since receiving their last over-the-counter provision of the gliflozin Sodium-Glucose Cotransport 2 inhibitor pharmaceutical composition, and
an indication of whether the subject has experienced a bacterial infection since receiving their last over-the-counter provision of the gliflozin Sodium-Glucose Cotransport 2 inhibitor pharmaceutical composition,
C) applying an algorithm to the reassessment information set, via a third computer system having a processor programed to perform the algorithm, wherein the algorithm:
i) runs all or a portion of the reassessment information set against a first set of reassessment filters, wherein, when a respective filter in the first set of reassessment filters is fired (1) the method is terminated without authorizing the over-the-counter provision of the gliflozin Sodium-Glucose Cotransport 2 inhibitor pharmaceutical composition to the subject or (2) the subject is provided with a warning corresponding to the respective filter, and wherein the first set of reassessment filters comprises:
when the subject is a female, a pregnancy reassessment filter that is fired at least when the reassessment information set indicates the subject is pregnant or breastfeeding,
a diabetes medication reassessment filter that is fired at least when the reassessment information set indicates the subject has started taking a medication for treating diabetes other than the gliflozin Sodium-Glucose Cotransport 2 inhibitor pharmaceutical composition since receiving their last over-the-counter provision of the gliflozin Sodium-Glucose Cotransport 2 inhibitor pharmaceutical composition,
a kidney disease reassessment filter that is fired at least when the reassessment information set indicates that the subject has developed kidney disease since receiving their last over-the-counter provision of the gliflozin Sodium-Glucose Cotransport 2 inhibitor pharmaceutical composition,
a urinary tract infection reassessment filter that is fired at least when the reassessment information set indicates that the subject has experienced symptoms of a urinary tract infection since receiving their last over-the-counter provision of the gliflozin Sodium-Glucose Cotransport 2 inhibitor pharmaceutical composition, and
a bacterial infection reassessment filter that is fired at least when the reassessment information set indicates that the subject has experienced symptoms of a bacterial infection since receiving their last over-the-counter provision of the gliflozin Sodium-Glucose Cotransport 2 inhibitor pharmaceutical composition,
ii) obtains, when (1) the method is not already terminated by firing of a filter in the plurality of filters and (2) a respective filter in the plurality of filters has fired, acknowledgment from the subject for each warning issued to the subject by any filter in the plurality of reassessment filters, and
iii) proceeds with a fulfillment process when (1) the method is not already terminated by the firing of a filter in the first set of reassessment filters and (2) the subject has acknowledged each warning associated with each filter in the first set of reassessment filters that was fired and that is associated with a warning, wherein the fulfillment process comprises:
storing an indication in a subject profile of a re-authorization for the gliflozin Sodium-Glucose Cotransport 2 inhibitor pharmaceutical composition using the processor of the third computer, and
re-authorizing the over-the-counter provision of the gliflozin Sodium-Glucose Cotransport 2 inhibitor pharmaceutical composition to the subject.

35. The method of claim 33 or 34, wherein the first set of reassessment filters further comprises a blood glucose reassessment filter that is fired at least when the reassessment information set indicates that the subject has a blood glucose level that is indicative of diabetes.

36. The method of claim 35, wherein the blood glucose reassessment filter is fired when the reassessment information set indicates the subject has an HbA1c level of greater than 6.4%.

37. The method of claim 35 or 36, wherein the blood glucose reassessment filter is fired when the reassessment information set indicates the subject has a fasting blood glucose level of greater than 125 mg/dL.

38. The method of any one of claims 35-37, wherein the blood glucose reassessment filter is fired when the reassessment information set indicates the subject has a blood glucose level of at least 200 mg/dL two hours after consuming 75 grams of glucose.

39. The method of any one of claims 34-38, wherein the algorithm bypasses the blood glucose reassessment filter when it is determined that less than a threshold amount of time has passed since the subject provided an indication of their blood glucose level.

40. The method of claim 39, wherein the threshold amount of time is twelve months.

41. The method of any one of claims 33-40, wherein the obtaining A) comprises providing a reassessment survey to the subject.

42. The method of any one of claims 33-41, wherein the pregnancy reassessment filter is fired when the reassessment information set indicates the subject is female and planning on becoming pregnant.

43. The method of any one of claims 33-42, wherein:
the indication of whether the subject has kidney disease in the reassessment information set includes whether the subject has been diagnosed with kidney disease, and
the kidney disease reassessment filter is fired at least when the reassessment information set indicates the subject has been diagnosed with kidney disease.

44. The method of any one of claims 33-43, wherein:
the indication of whether the subject has kidney disease in the reassessment information set includes whether the subject is receiving kidney dialysis, and
the kidney disease reassessment filter is fired at least when the reassessment information set indicates the subject is receiving kidney dialysis.

45. The method of any one of claims 33-44, wherein:
the indication of whether the subject has kidney disease in the reassessment information set includes whether the subject has experienced a symptom of a kidney problem since receiving their last over-the-counter provision of the gliflozin Sodium-Glucose Cotransport 2 inhibitor pharmaceutical composition, and
the kidney problem reassessment filter is fired at least when the reassessment information set indicates the subject has experienced a symptom of a kidney problem since receiving their last over-the-counter provision of the gliflozin Sodium-Glucose Cotransport 2 inhibitor pharmaceutical composition.

46. The method of claim 45, wherein the symptom of the kidney problems is selected from the group consisting of a reduction in food or liquid intake, vomiting, diarrhea, or dehydration.

47. The method of claim 43 or 46, further comprising, when the reassessment information set indicates the subject has experienced a symptom of a kidney problem since receiving their last over-the-counter provision of the gliflozin Sodium-Glucose Cotransport 2 inhibitor pharmaceutical composition, storing a record associated with the firing of the kidney disease reassessment filter in an adverse event profile comprising records of filter firing events associated with a plurality of subjects.

48. The method of any one of claims 33-47, wherein the urinary tract infection reassessment filter is fired at least when the reassessment information set indicates the subject has experienced a symptom of a urinary tract infection since receiving their last provision of the gliflozin Sodium-Glucose Cotransport 2 inhibitor pharmaceutical composition.

49. The method of claim 48, wherein the symptom of the urinary tract infection is selected from the group consisting of a burning feeling when passing urine, an increased need to urinate, a need to urinate immediately after ingesting liquids, pain in the lower stomach, and blood in the subject's urine.

50. The method of claim 48, further comprising, when the urinary tract infection reassessment filter is fired, storing a record associated with the firing of the urinary tract infection reassessment filter in an adverse event profile comprising records of filter firing events associated with a plurality of subjects.

51. The method of any one of claims 33-50, wherein the bacterial infection reassessment filter is fired when the reassessment information set indicates the subject has experienced an unexplained symptom of a bacterial infection since receiving their last over-the-counter provision of the gliflozin Sodium-Glucose Cotransport 2 inhibitor pharmaceutical composition, wherein the symptom of the bacterial infection is pain, tenderness, swelling, or redness around the anus and genitals with accompanying fever or weakness.

52. The method of claim 51, further comprising, when the bacterial infection reassessment filter is fired, storing a record associated with the firing of the bacterial infection reassessment filter in an adverse event profile comprising records of filter firing events associated with a plurality of subjects.

53. The method of any one of claims 33-52, wherein the indication of the re-order authorization stored in the subject profile is an indication of a re-order for the over-the-counter provision of the gliflozin Sodium-Glucose Cotransport 2 inhibitor pharmaceutical composition.

54. The method of any one of claims 33-53, wherein:
the fulfillment process further comprises communicating an over-the-counter drug facts label for the gliflozin Sodium-Glucose Cotransport 2 inhibitor pharmaceutical composition to the subject, and
the over-the-counter provision of the gliflozin Sodium-Glucose Cotransport 2 inhibitor pharmaceutical composition is authorized upon condition of receipt of confirmation from the subject that the over-the-counter drug facts label has been received and read.

55. The method of any one of claims 33-52, wherein the fulfillment process further comprises storing a destination associated with the subject in the subject profile.

56. The method of any one of claims 33-55, wherein the fulfillment process further comprises coordinating shipping of the gliflozin Sodium-Glucose Cotransport 2 inhibitor pharmaceutical composition to a physical address associated with the subject.

57. The method of any one of claims 33-56, wherein (i) the first computer system and the second computer system are the same computer system, (ii) the first computer system and the third computer system are the same computer system, (iii) the second computer system and the third computer system are the same computer system, or (iv) the first computer system, second computer system, and third computer system are the same computer system.

58. The method of any one of claims 1-21 and 34-57, wherein the warning associated with any one of the fired filters comprises a prompt for the subject to indicate whether they have discussed a risk factor underlying the respective filter that was fired with a health care provider; and
acknowledgement is obtained from the subject when the subject indicates that they have discussed the risk factor underlying the respective filter that was fired with a health care provider.

59. The method of any one of claims 1-58, wherein:
the gliflozin Sodium-Glucose Cotransport 2 inhibitor pharmaceutical composition comprises empagliflozin or ertugliflozin as an active ingredient,
the reassessment information set includes an indication of whether the subject's low-density lipoprotein cholesterol (LDL-C) level has increased since receiving their last over-the-counter provision of the gliflozin Sodium-Glucose Cotransport 2 inhibitor pharmaceutical composition, and
the first set of reassessment filters includes an LDL-C reassessment filter that is fired at least when the assessment information set includes an indication that the subject's LDL-C level has increased more than a threshold amount since receiving their last over-the-counter provision of the gliflozin Sodium-Glucose Cotransport 2 inhibitor pharmaceutical composition.

60. The method of any one of claims 1-58, wherein the gliflozin Sodium-Glucose Cotransport 2 inhibitor pharmaceutical composition comprises (2S,3R,4R,5S,6R)-2-[4-chloro-3-[(4-ethoxyphenyl)methyl]phenyl]-6-(hydroxymethyl)oxane-3,4,5-triol, or a pharmaceutically acceptable salt thereof, as an active ingredient.

61. The method of any one of claims 1-58, wherein the gliflozin Sodium-Glucose Cotransport 2 inhibitor pharmaceutical composition comprises an active ingredient having the structure: 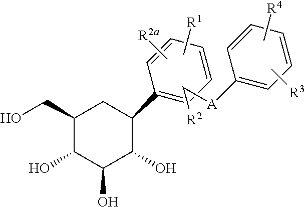
wherein
R¹, R² and R^{2a} are independently hydrogen, OH, OR⁵, alkyl, CF₃, OCHF₂, OCF₃, SR⁵ⁱ or halogen, or two of R¹, R² and R^{2a} together with the carbons to which they are attached can form an annelated five, six or seven membered carbocycle or heterocycle which may contain 1 to 4 heteroatoms in the ring which are N, O, S, SO, and/or SO₂;
R³ and R⁴ are independently hydrogen, OH, OR^{5a}, OAryl, OCH₂Aryl, alkyl, cycloalkyl, CF₃, —OCHF₂, —OCF₃, halogen, —CN, —CO₂R^{5b}, —CO₂H, COR^{6b}, —CH(OH)R^{6c}, —CH (OR^{5h})R^{6d}, —CONR⁶R^{6a}, —NHCOR^{5c}, —NHSO₂R^{5d}, —NHSO₂Aryl, Aryl, —SR^{5e}, —SOR^{5f}, —SO₂R^{5g}, —SO₂Aryl, or a five, six or seven membered heterocycle which may contain 1 to 4 heteroatoms in the ring which are N, O, S, SO, and/or SO₂, or R³ and R⁴ together with the carbons to which they are attached form an annelated five, six or seven membered carbocycle or heterocycle which may contain 1 to 4 heteroatoms in the ring which are N, O, S, SO, and/or SO₂;
R⁵, R^{5a}, R^{5b}, R^{5c}, R^{5d}, R^{5e}, R^{5f}, R^{5g}, R^{5h} and R⁵ⁱ are independently alkyl;
R⁶, R^{6a}, R^{6b}, R^{6c}, and R^{6d} are independently hydrogen, alkyl, aryl, alkylaryl or cycloalkyl, or R⁶ and R^{6a} together with the nitrogen to which they are attached form an annelated five, six or seven membered heterocycle which may contain 1 to 4 heteroatoms in the ring which are N, O, S, SO, and/or SO₂; and
A is O, S, NH, or (CH₂)ₙ, wherein n is 0-3, or a pharmaceutically acceptable salt, stereoisomer, or prodrug ester thereof;
with the proviso that when A is (CH₂)ₙ, wherein n is 0, 1, 2, or 3 or A is O, and at least one of R¹, R², and R^{2a} is OH or OR⁵, then at least one of R¹, R², and R^{2a} is CF₃, OCF₃, or OCHF₂ and/or at least one of R³ and R⁴ is CF₃, —OCHF₂, —OCF₃, —CN, —CO₂R^{5b} CH(OR^{5h})R^{6d}, CH(OH)R^{6c}, COR^{6b}, —NHCOR^{5c}, —NHSO₂R^{5d}, —NHSO₂Aryl, Aryl, —SR^{5e}, —SOR^{5f}, —SO₂R^{5g} or —SO₂Aryl.

62. The method of any one of claims 1-58, wherein the gliflozin Sodium-Glucose Cotransport 2 inhibitor pharmaceutical composition comprises dapagliflozin, or a pharmaceutically acceptable salt thereof, as an active ingredient.

63. The method of any one of claims 1-58, wherein the gliflozin Sodium-Glucose Cotransport 2 inhibitor pharmaceutical composition is dapagliflozin propanediol, as an active ingredient.

64. The method of any one of claims 60-63, wherein, upon confirmation from the subject that the over-the-counter drug facts label has been received and read, the subject is authorized for provision of a dosage of from 2.5 mg to 10 mg per day of the active ingredient.

65. The method of any one of claims 60-63, wherein, upon confirmation from the subject that the over-the-counter drug facts label has been received and read, the subject is authorized for provision of a dosage of 5 mg per day of the active ingredient.

66. The method of any one of claims 1-59, wherein the gliflozin Sodium-Glucose Cotransport 2 inhibitor pharmaceutical composition comprises an active ingredient selected from the group consisting of empagliflozin, canagliflozin, ertugliflozin, and a pharmaceutically acceptable salt thereof.

67. The method of any one of claims 1-58, wherein, upon confirmation from the subject that the over-the-counter drug facts label has been received and read, the subject is authorized for provision of a dosage of from 50 mg to 300 mg per day of canagliflozin.

68. The method of any one of claims 1-59, wherein, upon confirmation from the subject that the over-the-counter drug facts label has been received and read, the subject is authorized for provision of a dosage of from 5 mg to 25 mg per day of empagliflozin.

69. The method of any one of claims 1-59, wherein, upon confirmation from the subject that the over-the-counter drug facts label has been received and read, the subject is authorized for provision of a dosage of from 2.5 mg to 15 mg per day of ertugliflozin.

70. The method of any one of claims 1-69, further comprising administering, upon authorization of the over-the-counter provision, the gliflozin Sodium-Glucose Cotransport 2 inhibitor pharmaceutical composition to the subject as a treatment for prediabetes.

71. An electronic device, comprising:
one or more processors;
memory; and
one or more programs, wherein the one or more programs are stored in the memory and configured to be executed by the one or more processors, the one or more programs including instructions for performing a method according to any one of claims 1 to 70.

72. The electronic device of claim 71, wherein the one or more programs include instructions for:
(i) performing a method for authorizing a human subject for an over-the-counter provision of a gliflozin Sodium-Glucose Cotransport 2 inhibitor pharmaceutical composition according to any one of claims 1 to 21, and
(ii) performing a method for re-authorizing the human subject for an over-the-counter provision of the gliflozin Sodium-Glucose Cotransport 2 inhibitor pharmaceutical composition according to any one of claims 33 to 59.

73. A computer readable storage medium storing one or more programs, the one or more programs comprising instructions, which when executed by an electronic device with one or more processors and a memory, cause the device to perform a method according to any one of claims 1 to 70.

74. The computer readable storage medium of claim 73, wherein the one or more programs include:
(i) first instructions, which when executed by an electronic device with one or more processors and a memory, cause the device to perform a method for authorizing a human subject for an over-the-counter provision of a gliflozin Sodium-Glucose Cotransport 2 inhibitor pharmaceutical composition according to any one of claims 1 to 20, and
(ii) second instructions, which when executed by an electronic device with one or more processors and a memory, cause the device to perform a method for re-authorizing the human subject for an over-the-counter provision of the gliflozin Sodium-Glucose Cotransport 2 inhibitor composition according to any one of claims 33 to 59.
